(19) United States
(12) Patent Application Publication
(10) Pub. No.: US 2012/0141502 A1
(43) Pub. Date: Jun. 7, 2012

(54) ANTIBODIES SPECIFIC TO E6 PROTEINS OF HPV AND USE THEREOF

(76) Inventors: Eric Dixon, Cary, NC (US); Rainer Blaesius, Chapel Hill, NC (US); Stephen Simkins, Watertown, CT (US); Steven L. Knapp, Apex, NC (US); George Brough, Wake Forest, NC (US); Karen Lenz, Apex, NJ (US); Johannes Schweizer, San Jose, CA (US); Peter Lu, Pato Alto, CA (US); David Garman, Thornhill (CA); Jon Silver, San Jose, CA (US); Charles Mahoney, Sunnyvale, CA (US); Chamorro Somoza Diaz-Sarmiento, Santa Clara, CA (US)

(21) Appl. No.: 13/265,332

(22) PCT Filed: Apr. 20, 2010
(86) PCT No.: PCT/US10/01189

§ 371 Date: Feb. 3, 2012

Related U.S. Application Data
(60) Provisional application No. 61/171,039, filed on Apr. 20, 2009.
(60) Provisional application No. 61/171,032, filed on Apr. 20, 2009.
(60) Provisional application No. 61/171,025, filed on Apr. 20, 2009.
(60) Provisional application No. 61/175,362, filed on May 4, 2009.
(60) Provisional application No. 61/175,365, filed on May 4, 2009.

(51) Int. Cl. A61K 39/395; A61P 31/20; C40B 30/04; C12Q 1/70; C40B 40/10

(52) U.S. Cl. 424/159.1; 435/5; 506/18; 506/9

(57) ABSTRACT

The subject invention provides an antibody composition for detecting E6 protein of at least one HPV strain in a sample. The subject antibodies may be used to detect oncogenic HPV E6 proteins in a sample, and the antibodies find use in a variety of diagnostic and therapeutic applications, including methods of diagnosing and treating cancer. Kits for performing the subject methods and containing the subject antibodies are also provided. Also disclosed in the present invention is a method of generating an antibody that specifically binds to amino-terminus of E6 proteins of at least two HPV strains.

### RELATED APPLICATIONS

This application claims the benefit of U.S. Provisional Application No. 61/171,025, filed Apr. 20, 2009, U.S. Provisional Application No. 61/171,032, filed Apr. 20, 2009, U.S. Provisional Application No. 61/171,039, filed Apr. 20, 2009, U.S. Provisional Application No. 61/175,362, filed May 4, 2009, and U.S. Provisional Application No. 61/175,365, filed May 4, 2009, each of which is incorporated by reference herein in its entirety.

### BACKGROUND OF THE INVENTION

Cervical cancer is the second most common cancer diagnosis in women and is linked to high-risk human papillomavirus infection 99.7% of the time. Currently, 12,000 new cases of invasive cervical cancer are diagnosed in US women annually, resulting in 5,000 deaths each year. Furthermore, there are approximately 400,000 cases of cervical cancer and close to 200,000 deaths annually worldwide. Human papillomaviruses (HPVs) are one of the most common causes of sexually transmitted disease in the world. Overall, 50-75% of sexually active men and women acquire genital HPV infections at some point in their lives. An estimated 5.5 million people become infected with HPV each year in the US alone, and at least 20 million are currently infected. The more than 100 different isolates of HPV have been broadly subdivided into high-risk and low-risk subtypes based on their association with cervical carcinomas or with benign cervical lesions or dysplasias.

A number of lines of evidence point to HPV infections as the etiological agents of cervical cancers. Multiple studies in the 1980's reported the presence of HPV variants in cervical dysplasias, cancer, and in cell lines derived from cervical cancer. Further research demonstrated that the E6-E7 region of the genome from oncogenic HPV 18 is selectively retained in cervical cancer cells, suggesting that HPV infection could be causative and that continued expression of the E6-E7 region is required for maintenance of the immortalized or cancerous state. Further research demonstrated that the E6-E7 genes from HPV 16 are sufficient to immortalize human keratinocytes in culture. It was also demonstrated that although E6-E7 genes from high risk HPVs could transform cell lines, the E6-E7 regions from low risk, or non-oncogenic variants such as HPV 6 and HPV11 are unable to transform human keratinocytes. HPV 16 and 18 infection was examined by in situ hybridization and E6 protein expression by immunocytochemistry in 623 cervical tissue samples at various stages of tumor progression and a significant correlation was found between histological abnormality and HPV infection.

A significant unmet need exists for early and accurate diagnosis of oncogenic HPV infection as well as for treatments directed at the causative HPV infection, preventing the development of cervical cancer by intervening earlier in disease progression. Human papillomaviruses characterized to date are associated with lesions confined to the epithelial layers of skin, or oral, pharyngeal, respiratory, and, most importantly, anogenital mucosae. Specific human papillomavirus types, including HPV 6 and 11, frequently cause benign mucosal lesions, whereas other types such as HPV 16, 18, and a host of other strains, are predominantly found in high-grade lesions and cancer. Individual types of human papillomaviruses (HPV) which infect mucosal surfaces have been implicated as the causative agents for carcinomas of the cervix, breast (Yu et al. (1999) Anticancer Res. 19:55555057-5061; Liu et al. (2001) J. Hum. Virol. 44:329-334), anus, penis, prostate (De Villiers et al. (1989) Virology 171:248:253), larynx and the buccal cavity, tonsils (Snijders et al. (1994) J. Gen. Virol. 75(Pt 10):2769-2775), nasal passage (Trujillo et al. (1996) Virus Genes 12:165-178; Wu et al. (1993) Lancet 341:522-524), skin (Trenfield et al. (1993) Australas. J. Dermatol. 34:71-78), bladder (Baithun et al. (1998) Cancer Surv. 31:17-27), head and neck squamous-cell carcinomas (Braakhuis et al. (2004) J. Natl. Cancer Inst. 96:978-980), occasional periungal carcinomas, as well as benign anogenital warts. The identification of particular HPV types is used for identifying subjects with premalignant lesions who are at risk of progression to malignancy. Although visible anogenital lesions are present in some persons infected with human papillomavirus, the majority of individuals with HPV genital tract infection do not have clinically apparent disease, but analysis of cytomorphological traits present in cervical smears can be used to detect HPV infection. Papanicolaou tests are a valuable screening tool, but they miss a large proportion of HPV-infected persons due to the unfortunate false positive and false negative test results. In addition, they are not amenable to worldwide testing because interpretation of results requires trained pathologists.

HPV infection is also associated with Netherton's syndrome (Weber et al. (2001) Br. J. Dermatol. 144:1044-1049) and epidermolysis verruciformis (Rubaie et al. (1998) Int. J. Dermatol. 37:766-771). HPV can also be transmitted to a fetus by the mother (Smith et al. (2004) Sex. Transm. Dis. 31:57-62; Xu et al. (1998) Chin. Med. Sci. J. 13:29-31; Cason et al. (1998) Intervirology 41:213-218).

The detection and diagnosis of disease is a prerequisite for the treatment of disease. Numerous markers and characteristics of diseases have been identified and many are used for the diagnosis of disease. Many diseases are preceded by, and are characterized by, changes in the state of the affected cells. Changes can include the expression of pathogens or proteins in infected cells, changes in the expression patterns of genes or proteins in affected cells, and changes in cell morphology. The detection, diagnosis, and monitoring of diseases can be aided by the accurate assessment of these changes. Inexpensive, rapid, early and accurate detection of pathogens can allow treatment and prevention of diseases that range in effect from discomfort to death.

### Literature

Literature of interest includes the following references: Zozulya et al., (Genome Biology 2:0018.1-0018.12, 2001; Mombairts (Annu. Rev. Neurosci 22:487-509, 1999); Raining et al., (Nature 361: 353-356, 1993); Belluscio et al., (Neuron 20: 69-81, 1988); Ronnet et al., (Annu. Rev. Physiol. 64:189-222, 2002); Lu et al., (Traffic 4: 416-533, 2003); Buck (Cell 100:611-618, 2000); Malnic et al., (Cell 96:713-723, 1999); Firestein (Nature 413:211-218, 2001); Zhao et al., (Science 279: 237-242, 1998); Touhara et al., (Proc. Natl. Acad. Sci. 96: 4040-4045, 1999); Sklar et al., (J. Biol. Chem. 261:15538-15543, 1986); Dryer et al., (TiPS 20:413-417, 1999); Ivic et al., (J. Neurobiol. 50:56-68, 2002); Munger (2002) Front. Biosci. 7:d641-9; Glaunsinger (2000) Oncogene 19:5270-80; Gardiol (1999) Oncogene 18:5487-96; Pim (1999) Oncogene 18:7403-8; Meschede (1998) J. Clin. Microbiol. 36:475-80; Kiyono (1997) Proc. Natl. Acad. Sci. 94:11612-6; and Lee (1997) Proc. Natl. Acad. Sci. 94:6670-5; Banks (1987) J. Gen. Virol. 68:1351-1359; Fuchs et al., (Hum. Genet. 108:1-13, 2001); and Giovane et al. (1999) Journal of Molecular Recognition 12:141-152 and published US patent applications 20030143679 and 20030105285; and U.S. Pat. Nos. 6,610,511, 6,492,143 6,410,249, 6,322,794, 6,344,314, 5,415,995, 5,753,233, 5,876,723, 5,648,459, 6,391,539, 5,665,535 and 4,777,239.

### SUMMARY OF THE INVENTION

In one aspect, the present invention provides an antibody which specifically binds to amino-terminus (N-terminus) of oncognenic E6 proteins of at least two strains of human papilloma virus (HPV) with enhanced binding affinity and sensitivity of detecting the E6 proteins of at least two HPV strains. In some embodiments, the antibody specifically binds to E6 proteins of at least three different oncogenic HPV strains. In some embodiments, the antibody specifically binds to E6 proteins of HPV strains 16, 18, and 45. In some embodiments, the antibody specifically binds to E6 proteins of at least six different oncogenic HPV strains. In some embodiments, the antibody specifically binds to E6 proteins of HPV strains 16, 18, 31, 33, 45, 52, and 58. The antibody can also specifically bind to E6 proteins of HPV strains 16, 18, 26, 30, 31, 34, 39, 45, 51, 52, 53, 58, 59, 66, 68, 69, 70, 73, or 82 or a combination thereof. In some embodiments, the antibody specifically binds to E6 proteins in a sample. The sample can be a cervical scrape, cervical biopsy, cervical lavage, blood or urine. The sample can also be a histological sample. In some embodiments, the antibody binds to E6 protein with a binding affinity of less than 10⁻⁸ M, less than 10⁻⁹ M, less than 10⁻¹⁰ M, less than 10⁻¹¹ M, or less than 10⁻¹² M. In some embodiments, the antibody detects E6 protein with increased sensitivity. In some embodiments, the antibody is monoclonal. The antibody can also be labeled. In some embodiments, the antibody is a mixture of two or more monoclonal antibodies specific against oncogenic E6 proteins. In some embodiments, the antibody is used as a part of a test for cervical cancer. Also provided by the present invention is a kit for detection of an E6 protein of an oncogenic HPV strain in a sample, comprising the subject antibody disclosed herein. In some embodiments, the kit further comprises reagents for detection of the antibody. The detection can be by an enzyme-linked immunosorbent assay (ELISA).

In another aspect, the present invention provides a method of detecting an E6 protein of at least two HPV strains in a sample, comprising: contacting an antibody which specifically binds to amino-terminus (N-terminus) of E6 proteins of at least two HPV strains with the sample; and detecting any binding of the antibody to the E6 protein in the sample; wherein binding of the antibody to the E6 protein in the sample indicates the presence of at least one HPV strain in the sample; and wherein the binding affinity of the antibody to the E6 protein is increased. In some embodiments of the subject method, the antibody specifically binds to E6 proteins of at least three different oncogenic HPV strains. In some embodiments, the antibody specifically binds to E6 proteins of HPV strains 16, 18, and 45. In some embodiments, the antibody specifically binds to E6 proteins of at least six different oncogenic HPV strains. The antibody may specifically bind to E6 proteins of HPV strain 16, 18, 26, 30, 31, 34, 39, 45, 51, 52, 53, 58, 59, 66, 68, 69, 70, 73, or 82 or a combination thereof. In some embodiments of the subject method, the sample is a cervical scrape, cervical biopsy, cervical lavage, blood or urine. The sample can be a histological sample. In some embodiments, the sample is from a human. In some embodiments, the antibody binds to E6 protein with a binding affinity of less than 10⁻⁸ M, less than 10⁻⁹ M, less than 10⁻¹⁰ M, less than 10⁻¹¹ M, or less than 10⁻¹² M. In some embodiments of the subject method, the antibody detects E6 protein with increased sensitivity. The antibody can be monoclonal or labeled. In some embodiments, the antibody is used as a part of a test for cervical cancer. In some embodiments, the antibody is a mixture of two or more monoclonal antibodies specific against oncogenic E6 proteins. In some embodiments, the antibody is used as a capture antibody to capture E6 protein in an enzyme-linked immunosorbent assay (ELISA).

In some embodiments, the subject method is an enzyme-linked immunosorbent assay (ELISA), comprising: contacting the subject antibody with the sample; contacting the E6 protein that is bound to the subject antibody with another E6 binding partner that specifically binds to the E6 protein at a binding site that is different from that of the subject antibody; and detecting binding of the E6 binding partner to the E6 protein, thereby detecting the presence of the E6 protein in the sample. In some embodiments, the antibody is used as a detector antibody to detect E6 protein that is bound to an E6 binding partner specific for the E6 protein in an enzyme-linked immunosorbent assay (ELISA). In some embodiments, the method is an enzyme-linked immunosorbent assay (ELISA), comprising: contacting the sample with an E6 binding partner that specifically binds to E6 protein at a binding site that is different from that of the subject antibody; contacting the E6 protein that is bound to the E6 binding partner with the subject antibody; and detecting binding of the subject antibody to the E6 protein, thereby detecting the presence of the E6 protein in the sample. In some embodiments, the detection of E6 protein is via an immunological based assay selected from the group consisting of enzyme immunoassays (EIA), Ramon spectroscopy, lateral flow, and cytometric bead array (CBA). In some embodiments, the antibody is immobilized. In some embodiments, the E6 binding partner is immobilized. In some embodiments, the E6 binding partner is a PDZ domain containing polypeptide. The E6 binding partner can be ah antibody specific against the E6 protein. In some embodiments, the antibody is used in combination with an antibody that specifically binds to C-terminal region of an E6 protein for detection of the E6 protein. In some embodiments, the antibody is used in combination with a PDZ domain containing polypeptide that binds to C-terminal region of an E6 protein for detection of the E6 protein.

In yet another aspect, the present invention provides a method of generating an antibody that binds to amino-terminus (N-terminus) of E6 proteins of at least two HPV strains in a sample, the method comprising: (a) immunizing animal with a peptide which has a T cell epitope sequence fused with an N-terminal sequence of an E6 protein; (b) obtaining B lymphocytes from the immunized animal; (c) fusing the B lymphocytes obtained from the immunized animal with myeloma cells to generate hybridoma cells secreting antibodies; and (d) screening the hybridoma cells for antibodies that specifically bind to the N-terminus of E6 proteins of at least two HPV strains. In some embodiments, the T cell epitope amino acid sequence is F-I-S-E-A-I-I-H-V-L-H-S-R. In some embodiments, the immunizing peptide is a consensus peptide present in HPV-16 E6 protein. In some embodiments, the immunizing peptide is a consensus peptide present in HPV-18 E6 protein. In some embodiments, the immunizing peptide step contains an amino acid sequence F-Q-D-P-A-E-R-P-R-K-L-H-D-L-C-T-E-L or F-Q-D-P-A-E-R-P-Y-K-L-P-D-L-C-T-E-L. In some embodiments, the method further comprises cloning hybridoma cells that secret antibodies specific for the N-terminus of oncognenic E6 proteins. In some embodiments, the method further comprises purifying antibodies that specifically bind to the N-terminus of oncognenic E6 proteins. In some embodiments of the subject method, the antibody is monoclonal. In some embodiments, the antibody specifically binds to E6 proteins of at least three different oncogenic HPV strains. In some embodiments, the antibody specifically binds to E6 proteins of HPV strains 16, 18, and 45. In some embodiments, the antibody specifically binds to E6 proteins of at least six different oncogenic HPV strains. In some embodiments, the antibody specifically binds to E6 proteins of HPV strains 16, 18, 26, 30, 31, 34, 39, 45, 51, 52, 53, 58, 59, 66, 68, 69, 70, 73, or 82 or a combination thereof. In some embodiments of the subject method, the sample is a cervical scrape, cervical biopsy, cervical lavage, blood or urine, or a histological sample. In some embodiments, the antibody is used as a diagnostic or therapeutic agent for cervical cancer. In some embodiments, the antibody binds to E6 protein with a binding affinity of less than 10⁻⁸ M, less than 10⁻⁹M, less than 10⁻¹⁰ M, less than 10⁻¹¹ M, or less than 10⁻¹² M. In some embodiments, the antibody detects E6 protein with higher sensitivity.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1a and 1b show peptide antigens that closely mimic sequences of high-risk HPV for generating antibodies that are specific against N-terminal region of E6 proteins of at least two high-risk HPV strains. Black dots denote residues in all oncogenic strains and gray dots denote positions with only conservative differences. The differences between the two consensus peptide sequences shown in FIGS. 1a and 1b are highlighted in stripes.

FIG. 2 shows cross-reactivity of consensus peptide antibodies to HPV E6 types in ELISA. Recombinant HPV E6 proteins were purified and coated directly to microtiter plates (gray bars) or captured with a PDZ domain containing protein (black bars). Primary antibodies to the consensus peptides were diluted to 1 μg/ml and added to the wells. Binding was detected by addition of a secondary goat anti-mouse IgG:HRP followed by the substrate TMB. Signal to noise (S/N) ratios were calculated by dividing the OD₄₅₀ of test wells by the OD₄₅₀ from wells with no consensus peptide antibody. Cross-reactivity profiles varied greatly between antibodies. Clone 6H5.3 demonstrated monospecific binding, while clone 4E9.7 had the ability to bind 8 HPV E6 types in the direct ELISA format.

FIG. 3 shows cross-reactivity of consensus peptide antibodies to HPV E6 types in Western blot. Recombinant HPV E6 proteins were resolved by SDS-PAGE. Western blots were probed with consensus peptide antibodies diluted to 1 μg/ml in blocking buffer. Goat anti-mouse IgG:AP was used to detect binding. Antibody cross-reactivity profiles ranged from single type specific to 5 or more HPV types. Cross-reactivity to low risk HPV types 6b and 11 was not observed.

FIG. 4 shows immunoprecipitation of recombinant HPV16 E6 by consensus peptide antibodies. Antibodies were linked to protein-G Dynabeads and incubated with 1 μg of recombinant maltose binding protein (MBP) tagged HPV-16 E6 (˜60 kDa). After washing, immune complexes were separated by SDS-PAGE followed by Western blotting with an HPV-16 E6 specific mouse antibody. An alkaline phosphatase conjugated anti-mouse light chain specific antibody was used to detect immunoprecipitated HPV16 E6:MBP. Mouse Ig light chain is indicated by “LC”. Clone 4E9.7 was able to immunoprecipitate detectable levels of HPV16 E6:MBP.

FIG. 5 shows detection of HPV-16 E6 from SiHa cell lysates by sandwich ELISA using a consensus peptide capture antibody. HPV-16 positive SiHa cells were lysed in RIPA buffer and applied to microtiter plates according to cell equivalents (black circles). Consensus peptide antibody 4E9.7 was used as the capture antibody with an HPV-16 E6 specific monoclonal detector antibody. Lysates were prepared similarly for the HPV negative C33A− cell line (gray squares). HPV-16 E6 was detected from less than 5,000 SiHa cell equivalents.

FIG. 6 shows Western blot probed with HPV oncopeptide and anti-HIS monoclonal antibodies demonstrating the specificity of the antibody of the present invention for the HPV E6 oncopeptide.

FIG. 7 shows characterization of an anti-HPV 16 and 18 E6 monoclonal antibody by ELISA and Western blot. An intrasplenic immunization with T cell epitope-6mer peptides and CD40 agonist treatment resulted in a 5mer (RRETQ)-specific Mab. This Mab (2H9.15) was epitope mapped with a high degree of specificity to the C-terminus of oncogenic E6 proteins from HPV 16 and HPV 18. 2H9.15 has a potential utility as a pan-antibody that detects both HPV types 16 and 18.

FIG. 8 shows inhibition of HPV E6 binding to the MAGI-1 PDZ binding domain by competitive blocking with the C-terminal HPV E6 oncopeptide antibodies.

FIG. 9 shows sandwich ELISA pairings. Various anti-HPV E6 capture antibodies were used to develop sandwich ELISA assays to detect HPV16 E6 protein. Combining antibody libraries (capture C1, C2, and C3 MAb) with the HPV Oncopeptide PDZ antibodies identified capture/detector pairs.

FIG. 10 shows immunocytochemistry (ICC) for the detection of HPV E6 in cancer cell lines. In ICC, the MAb stained SiHa and HeLa cells expressing HPV E6 but not the HPV-negative cell line C-33A. Shown is the staining with MAb 1A9.1.

FIG. 11 is a scheme showing detection of E6 protein via anti-E6 antibody sandwich assay in comparison with PDZ peptide capture of E6.

FIG. 12 shows HPV16 singleplex detection. For HPV16-E6, the anti-E6 antibody sandwich assay results in an improved signal to background ratio and decreased dampening with individual cervical swab sample as compared to the PDZ peptide capture of E6.

FIG. 13 shows HPV16 singleplex detection in negative cervical swab samples (NCLS): comparison of anti-E6 mAb sandwich detection with PDZ peptide capture of E6.

FIG. 14 shows HPV18 singleplex detection using the anti-E6 mAb sandwich assay as compared to PDZ peptide capture of E6. The results show that for HPV18-E6, antibody capture results in substantially improved signal-to-background ratio.

FIG. 15 shows HPV45 singleplex detection in the presence of cervical swab material. The anti-E6 antibody sandwich assay results in improved signal-to-background ratio as compared to PDZ peptide capture of E6. No NCLS specific dampening was observed.

FIG. 16 is a scheme showing detection of E6 from multiple HPV strains on a test strip. E6 capture via HPV type specific anti-E6 mAb sandwich detection allows for E6 typing.

FIG. 17 shows HPV16+HPV18 multiplex detection via two test-line strip. E6 capture via HPV strain specific mAb allows for E6 typing and the HPV16/18 antibody detector cocktail does not result in higher background or reduced signal when compared to singleplex detection.

FIG. 18 shows HPV16+HPV18 multiplex detection via two test-line strip in negative cervical swab samples (NCLS).

FIG. 19 shows HPV16/18-E6 detection using a two test-line strip. The results show no HPV16/18 false positives on 60 individual HPV negative cervical swab samples (NCLS), indicating a low false positive rate using the anti-E6 antibody sandwich strip test approach.

FIG. 20 shows HPV16/18/45-E6 detection using a three test-line strip. The results show that E6 proteins from three different HPV strains can be detected as three distinct lines simultaneously on one strip.

### DETAILED DESCRIPTION OF THE INVENTION

Throughout this application, various publications, patents and published patent applications are cited. The disclosures of these publications, patents and published patent applications referenced in this application are hereby incorporated by reference in their entirety into the present disclosure. Citation herein by Applicant of a publication, patent, or published patent application is not an admission by Applicant of said publication, patent, or published patent application as prior art.

In one aspect, the present invention provides an antibody which specifically binds to the amino-terminus (N-terminus) of oncognenic E6 proteins of at least two strains of human papilloma virus (HPV). In some embodiments, the antibody specifically binds to E6 proteins of at least three different oncogenic HPV strains, for example, HPV strains 16, 18, and 45. In some embodiments, the antibody specifically binds to E6 proteins of at least six different oncogenic HPV strains, for example, HPV strains 16, 18, 31, 33, 45, 52, and 58. In some embodiments, the antibody binds to E6 protein of at least one HPV strain, preferably, 2, 3, 4, 5, 6, 7, 8, 9, 10, or more than 10 HPV strains, with a binding affinity of less than 10⁻⁸ M, 10⁻⁹ M, 10⁻¹⁰ M, 10⁻¹¹M, or 10⁻¹² M. In some embodiments, the antibody is monoclonal. The antibody may also be labeled. In some embodiments, the antibody is a mixture of two or more monoclonal antibodies specific against oncogenic E6 proteins.

In another aspect, the present invention provides a method of detecting an E6 protein of at least two HPV strains in a sample, comprising: contacting an antibody which specifically binds to amino-terminus (N-terminus) of E6 proteins of at least two HPV strains with the sample; and detecting any binding of the antibody to the E6 protein in the sample. In most embodiments, binding of the antibody to the E6 protein in the sample indicates the presence of at least one HPV strain in the sample. The sample may be a cervical scrape, cervical biopsy, cervical lavage, blood or urine. The sample may also be a histological sample. In some embodiments, the antibody is used as a′ capture antibody to capture E6 protein in an enzyme-linked immunosorbent assay (ELISA). In this case, the method of the present invention can be an enzyme-linked immunosorbent assay (ELISA), comprising: contacting an antibody of the present invention with the sample; contacting the E6 protein that is bound to the subject antibody with another E6-binding partner that specifically binds to the E6 protein at a binding site that is different from that of the antibody of the present invention; and detecting binding of the E6-binding partner to the E6 protein, thereby detecting the presence of the E6 protein in the sample. In other embodiments, the antibody is used as a detector antibody to detect E6 protein that is bound to an immobilized E6-binding partner specific for the E6 protein in an ELISA. For example, the method of the present invention may be an ELISA comprising: contacting the sample with an immobilized E6-binding partner that specifically binds to E6 protein at a binding site that is different from that of the antibody of the present invention; contacting the E6 protein that is bound to the immobilized E6-binding partner with an antibody of the present invention; and detecting binding of the subject antibody to the E6 protein, thereby detecting the presence of the E6 protein in the sample.

In yet another aspect, the present invention a method of generating an antibody that binds to amino-terminus (N-terminus) of E6 proteins of at least two HPV strains in a sample, the method comprising: (a) immunizing animal with a peptide which has a T cell epitope sequence fused with a C-terminal sequence of oncognenic E6 protein; (b) obtaining B lymphocytes from the immunized animal; (c) fusing the B lymphocytes obtained from the immunized animal with myeloma cells to generate hybridoma cells secreting antibodies; and (d) screening the hybridoma cells for antibodies that specifically bind to the N-terminus of oncognenic E6 proteins of at least two HPV strains. In some embodiments, the T cell epitope amino acid sequence is F-J-S-E-A-I-I-H-V-L-H-S-R.

In some embodiments, a consensus peptide is used to immunize an animal for production of the antibodies of the present invention. A consensus peptide disclosed herein can be used to generate antibodies that may cross-react with E6 proteins from more than one HPV strain with high affinity. Method of generating consensus peptide based on amino acid sequences is known in the art and may involve comparison and substitution of polar and/or nonpolar residues. The immunizing peptide of the present invention can be a consensus peptide of any length based on any portion within the N-terminal region of a HPV E6 protein. The consensus peptide can be based on the N-terminal region of 2, 3, 4, 5, 6, 7, 8, 9, 10, or more than 10 HPV strains, preferably oncogenic HPV strains. In some embodiments, the consensus peptide is based on E6 N-terminal region of HPV-16. In other embodiments, the consensus peptide is based on E6 N-terminal region of HPV-18. Examples of the immunizing peptides include amino acid sequences F-Q-D-P-A-E-R-P-R-K-L-H-D-L-C-T-E-L and F-Q-D-P-A-E-R-P-Y-K-L-P-D-L-C-T-E-L. The generated antibody of the present invention may bind to E6 proteins of at least three different oncogenic HPV strains, for example, HPV strains 16, 18, and 45. In some embodiments, the antibody generated via the method of the present invention specifically binds to E6 proteins of at least six different oncogenic HPV strains including but not limited to HPV 16, 18, 26, 30, 31, 34, 39, 45, 51, 52, 53, 58, 59, 66, 68, 69, 70, 73, and 82. In terms of the binding affinity of the subject antibody for E6 protein, the antibody may bind to E6 protein with a binding affinity of less than 10⁻⁸ M, less than 10⁻⁹M, less than 10⁻¹⁰ M, less than 10⁻¹¹ M, or less than 10⁻¹² M.

In one aspect, the present invention provides an antibody which specifically binds to the carboxyl-terminus (C-terminus) of oncogenic E6 proteins of at least two high-risk strains of human papilloma virus (HPV). In some embodiments, the antibody specifically binds to E6 proteins of at least three different oncogenic HPV strains, for example, HPV strains 16, 18, and 45. In some embodiments, the antibody specifically binds to E6 proteins of at least six different oncogenic HPV strains, for example, HPV strains 16, 18, 31, 33, 45, 52, and 58. In some embodiments, the antibody binds to E6 protein with a binding affinity of less than 10⁻⁸ M, 10⁻⁹ M, 10⁻¹⁰ M, 10⁻¹¹ M, or 10⁻¹² M. In some embodiments, the antibody is monoclonal. The antibody may also be labeled. In some embodiments, the antibody is a mixture of two or more monoclonal antibodies specific against oncogenic E6 proteins.

In another aspect, the present invention provides a method of detecting an E6 protein of an oncogenic HPV strain in a sample, comprising: contacting an antibody which specifically binds to carboxyl-terminus (C-terminus) of oncognenic E6 proteins of at least two HPV strains with the sample; and detecting any binding of the antibody to the E6 protein in the sample. In most embodiments, binding of the antibody to the E6 protein in the sample indicates the presence of at least one oncogenic HPV strain in the sample. The sample may be a cervical scrape, cervical biopsy, cervical lavage, blood or urine. The sample may also be a histological sample. In some embodiments, the antibody is used as an antibody to capture E6 protein in an enzyme-linked immunosorbent assay (ELISA). In this case, the method of the present invention can be an enzyme-linked immunosorbent assay (ELISA), comprising: contacting an antibody of the present invention with the sample; contacting the E6 protein that is bound to the subject antibody with a second antibody that specifically binds to the E6 protein at a binding site that is different from that of the antibody of the present invention; and detecting binding of the second antibody to the E6 protein, thereby detecting the presence of the E6 protein in the sample. In other embodiments, the antibody is used as a detector antibody to detect E6 protein that is bound to another E6 binding partner in an ELISA. An “E6 protein binding partner” can be any molecule that specifically binds to an oncogenic E6 protein. Suitable oncogenic E6 protein binding partners include a PDZ domain polypeptide (as described below), other antibodies against oncogenic E6 proteins (such as those described below); other proteins that recognize oncogenic E6 protein (e.g., p53, E6-AP or E6-BP); DNA (i.e., cruciform DNA); and other binding partners such as aptamers. In some embodiments, detection of more than one oncogenic E6 protein (e.g., all oncogenic E6 proteins, E6 proteins from HPV strains 16 and 18, or E6 proteins from HPV strains 16 and 45 etc.) is desirable, and, as such, an oncogenic E6 protein binding partner may be an antibody that binds to these proteins, as described below, or a mixture of antibodies that each binds to different oncogenic HPV E6 proteins. As is known in the art, such binding partners may be labeled to facilitate their detection. In general, binding partners bind E6 with a binding affinity of less then 10⁻⁵ M, e.g., less than 10⁻⁶, less than 10⁻⁷ M, less than 10⁻⁸ M (e.g., less than 10⁻⁹ M, 10⁻¹⁰, 10⁻¹¹, 10⁻¹² etc.).

In some examples, the method of the present invention may be an ELISA comprising: contacting the sample with a binding partner that specifically binds to E6 protein at a binding site that is different from that of the antibody of the present invention; contacting the E6 protein that is bound to the antibody with an antibody of the present invention; and detecting binding of the antibody of the present invention to the E6 protein, thereby detecting the presence of the E6 protein in the sample. The subject antibody may or may not be immobilized to a substrate in practicing the subject method.

In yet another aspect, the present invention a method of generating an antibody that binds to carboxyl-terminus (C-terminus) of oncognenic E6 proteins of at least two HPV strains, the method comprising: (a) immunizing an animal with chimeric peptide, which contains a T cell epitope sequence fused with a C-terminal sequence of oncognenic E6 protein; (b) obtaining B lymphocytes from the immunized animal; (c) fusing the B lymphocytes obtained from the immunized animal with myeloma cells to generate hybridoma cells secreting antibodies; and (d) screening the hybridoma cells for antibodies that specifically bind to the C-terminus of oncognenic E6 proteins of at least two oncogenic HPV strains. An example of a T cell epitope amino acid sequence is F-J-S-E-A-I-I-H-V-L-H-S-R. In some embodiments, the C-terminal sequence of an oncognenic E6 protein used in the immunizing step contains a PDZ domain binding motif. In some embodiments, the C-terminal sequence of an oncogenic E6 protein used in the immunizing step contains a conserved amino acid motif E-(T/S)-X-(V/L), i.e. the C-terminal consensus sequence. Examples of the consensus C-terminal sequence of an oncognenic E6 protein used in the immunizing step include amino acid sequences E-T-Q-L and E-T-Q-V. The generated antibody of the present invention may bind to E6 proteins of at least three different oncogenic HPV strains, for example, HPV strains 16, 18, and 45. In some embodiments, the antibody generated via the method of the present invention specifically binds to E6 proteins of at least six different oncogenic HPV strains including but not limited to HPV 16, 18, 26, 30, 31, 34, 39, 45, 51, 52, 53, 58, 59, 66, 68, 69, 70, 73, and 82.

In one aspect, the present invention provides an antibody composition for detecting E6 protein of at least one HPV strain in a sample, the composition comprising a first antibody and a second antibody, wherein the first antibody binds to E6 protein of an oncogenic HPV strain in the sample, and the second antibody specifically binds to the E6 protein that is bound by the first antibody, and wherein the second antibody is part of a signal producing system for detection of the E6 protein in the sample. In some embodiments, the first antibody is immobilized to a substrate. In other embodiments, the first antibody may be fused or bound to another molecule that is immobilized to a substrate. In some embodiments, the antibodies including polyclonal and monoclonal antibodies bind to E6 proteins from at least one strain of HPV. In some embodiments, the HPV strain is an oncogenic HPV strain. In some embodiments, the HPV strain is a non-oncogenic HPV strain. In other embodiments, the antibodies bind to E6 proteins from more than one oncogenic strain of HPV. In some embodiments, the antibodies specific for E6 proteins bind to amino acid motifs that are conserved between the E6 proteins of different HPV strains, particularly HPV strains 16 and 18. In another aspect, the subject antibodies may be used in a method as described herein, for example, an antibody sandwich binding assay, to detect E6 protein of an oncogenic HPV strain in a sample.

Accordingly, the antibodies of the present invention find use in a variety of diagnostic applications, including methods of diagnosing cancer, particularly cervical cancer. In another aspect of the present invention, kits for performing the subject methods and containing the subject antibodies are also provided.

Human Papilloma Virus (HPV) and E6 Oncoprotein

Human papillomaviruses (HPVs) are small double-stranded DNA viruses that induce hyperproliferative lesions in epithelial tissues. Genomic organization is a well conserved feature among papillomaviruses. There are three main regions in an HPV genome-early, late and the long control regions. In the early region (E) resides the transformation and immortalization potential of HPVs and consists of a number of regulatory genes for viral transcription and replication and cell cycle control. The late region (L) codes for the two capsid genes and the long control region (LCR) contains all the cis-regulatory elements necessary for HPV transcription including the early promoter and the origin of replication (ori). The HPV genome encodes 6 early (E) and two late (L) proteins.

E1 and E2 are the two viral proteins that are required for viral DNA replication, together with the host cell DNA replication machinery. E4 and E5 are needed for amplification of the viral genome in the upper layers of the epithelium. E6 and E7 proteins of high-risk HPV types are oncogenic. They cooperate to immortalize cells and also induce genomic instability. E6 and E7 abrogate the activity of the cellular tumor suppressor proteins p53 and Rb, respectively. E6 also increases telomerase activity. L1 and L2 proteins form the viral capsid and are expressed late in infection in the upper layers of the epithelium. The long-control-region (LCR) contains most of the regulatory DNA sequences needed for proper replication of the viral genome (origin of DNA replication) and for the expression of the viral genes (enhancer and promoter regions).

There are over 100 different types of HPV, and these HPV types i.e. strains have been separated into those that are more likely to develop into cancer and those that are less likely. The so-called “high risk” HPV types are more likely to lead to the development of cancer, while “low-risk” viruses rarely develop into cancer. Certain “high-risk” HPV strains infect epithelia in the anogenital region and are the etiological agents of cervical cancers. These high-risk HPV strains include but are not limited to HPV-16, HPV-18, HPV-26, HPV-30, HPV-31, HPV-33, HPV-35, HPV-39, HPV-45, HPV-51, HPV-52, HPV53, HPV54, HPV-56, HPV-58, HPV-59, HPV-66, HPV-68, HPV-69, HPV-73, and HPV-82. The “low-risk” HPV strains include but are not limited to HPV 6, 11, 40, 42, 43, 44, 54, 61, 70, 72, and 81. The sequence analysis of HPV E6 proteins from various HPV strains with regard to the oncogenic potential of the E6 proteins is shown in U.S. Pat. Nos. 7,312,041 and 7,399,467, both of which are herein incorporated by reference in their entirety.

An “oncogenic HPV strain” is an HPV strain that is known to cause cervical cancer as determined by the National Cancer Institute (NCI, 2001). “Oncogenic E6 proteins” are E6 proteins encoded by the above oncogenic HPV strains. The sequences of exemplary oncogenic E6 proteins of interest are disclosed in U.S. Pat. No. 7,399,467, which is herein incorporated by reference in its entirety. The sequences of various HPV proteins are also found as database entries at NCBI's Genbank database, as follows: HPV16-E6: GI:9627100; HPV18-E6: GI:9626069; HPV31-E6: GI:9627109; HPV35-E6: GI:9627127; HPV30-E6: GI:9627320; HPV39-E6: GI:9627165; HPV45-E6: GI:9627356; HPV51-E6: GI:9627155; HPV52-E6: GI:9627370; HPV56-E6: GI:9627383; HPV59-E6: GI:9627962; HPV58-E6: GI:9626489; HPV33-E6: GI:9627118; HPV66-E6: GI:9628582; HPV68b-E6: GI:184383; HPV69-E6: GI:9634605; HPV26-E6: GI:396956; HPV53-E6: GI:9627377; HPV73: GI:1491692; HPV82: GI:9634614, HPV34 GI:396989; HPV67 GI:3228267; and HPV70 GI:1173493.

The oncogenic potential of these high-risk HPV strains is dependent on the cooperative action of the two early viral gene products, E6 and E7, which bind and alter the activity of cell cycle-regulatory proteins. E6 gene encodes for a small nuclear protein product of about 16-19 kD (Greenfield, I., et al. (1991) Proc. Natl. Acad. Sci. U.S.A. 88, 11217-11221). E6 is found and expressed in all HPV-containing cells (Smotkin, D. & Wettstein, F. O. (1986) Proc. Natl. Acad. Sci. U.S.A. 83, 4680-4684). The E6 protein contributes most significantly to the malignant conversion of the basal layer of the cervical epithelium. The E6 product from high-risk HPV-16 and 18 interacts with the antioncogenic regulator p53 and the ubiquitin degradation pathway protein E6AP, leading to the degradation of the p53 protein (Werness, B. A., et al. (1990) Science 248, 76-79). Briefly, E6 forms a ternary complex composed of the tumor suppressor protein p53 and E6AP (E6-associated protein), a member of E3 ubiquitin ligase family of proteins, resulting in the ubiquitination and subsequent degradation of p53 (Huibregtse, J. M., et al. 1991. EMBO J. 10:4129-4135). Low-risk HPV types 6 and 11 E6 protein does not induce p53 degradation correlating with their weak transformation potential. Absence of functional p53 protein makes the cell highly susceptible to DNA damage and prevents the activation of p53-mediated apoptosis. Most HPV-positive tumors have wild-type p53 whereas HPV-negative tumors contain mutant p53 (Crook, T., et al. (1991) Oncogene 6, 873-875). As a result of the activities of the E6 protein, keratinocytes reactivate DNA synthesis and this in turn alters the growth and differentiation of the basal epithelium anogenital mucosa, resulting in their immortalization.

E6 forms a ternary complex composed of the tumor suppressor protein p53 and E6AP (E6-associated protein), a member of E3 ubiquitin ligase family of proteins, resulting in the ubiquitination and subsequent degradation of p53 (Huibregtse, J. M., et al. 1991. EMBO J. 10:4129-4135). E7 binds to and inactivates the retinoblastoma (pRb) family of proteins, thereby alleviating the pRb-mediated repression of E2F transcription factors that are responsible for transactivating many genes involved in progression into S phase (Cheng, S., et al. 1995. Genes Dev. 9:2335-2349). Selective retention and expression of these two viral oncoproteins is essential for HPV-induced oncogenesis (Androphy, E. J., et al. 1987. EMBO J. 6:989-992).

HPV-16 E6 gene has two alternative splicing sites resulting in the production of two additional protein products named E6*I and E6*II. However, only the full-length E6 has the capacity to interact with p53 and thus is the only one with clinical relevance. The E6 protein contains four Cys-X-X-Cys motifs forming zinc-binding structures similar to those present in several transcription factors (Grossman, S. R. & Laimins, L. A. (1989) Oncogene 4, 1089-1093).

Exemplary PDZ domain-containing proteins and PDZ domain sequences may be found in U.S. Pat. Nos. 7,312,041, and 7,399,467, which are herein incorporated by their entirety. The term “PDZ domain” also encompasses variants (e.g., naturally occurring variants) of the sequences (e.g., polymorphic variants, variants with conservative substitutions, and the like) and domains from alternative species (e.g. mouse, rat). Typically, PDZ domains are substantially identical to those shown in U.S. patent application Ser. Nos. 09/724,553 and 10/938,249), e.g., at least about 70%, at least about 80%, or at least about 90% amino acid residue identity when compared and aligned for maximum correspondence. It is appreciated in the art that PDZ domains can be mutated to give amino acid changes that can strengthen or weaken binding and to alter specificity, yet they remain PDZ domains (Schneider et al., 1998, Nat. Biotech. 17:170-5). Unless otherwise indicated, a reference to a particular PDZ domain (e.g. a MAGI-1 domain 2) is intended to encompass the particular PDZ domain and HPV E6-binding variants thereof. In other words, if a reference is made to a particular PDZ domain, a reference is also made to variants of that PDZ domain that bind oncogenic E6 protein of HPV, as described below. In this respect it is noted that the numbering of PDZ domains in a protein may change. For example, the MAGI-1 domain 2, as referenced herein, may be referenced as MAGI-1 domain 1 in other literature. As such, when a particular PDZ domain of a protein is referenced in this application, this reference should be understood in view of the sequence of that domain, as described herein, particularly in the sequence listing. U.S. Pat. Nos. 7,312,041, and 7,399,467 show the sequences, the names and Genbank accession numbers for various PDZ domains, where appropriate. Further description of PDZ proteins, particularly a description of MAGI-1 domain 2 protein, is found in Ser. No. 10/630,590, filed Jul. 29, 2003 and published as US20040018487. This publication is incorporated by reference herein in its entirety for all purposes.

In the case of the PDZ domains described herein, a “HPV E6-binding variant” of a particular PDZ domain is a PDZ domain variant that retains HPV E6 PDZ ligand binding activity. Assays for determining whether a PDZ domain variant binds HPV E6 are described in great detail below, and guidance for identifying which amino acids to change in a specific PDZ domain to make it into a variant may be found in a variety of sources. In one example, a PDZ domain may be compared to other PDZ domains described herein and amino acids at corresponding positions may be substituted, for example. In another example, the sequence a PDZ domain of a particular PDZ protein may be compared to the sequence of an equivalent PDZ domain in an equivalent PDZ protein from another species. For example, the sequence of a PDZ domain from a human PDZ protein may be compared to the sequence of other known and equivalent PDZ domains from other species (e.g., mouse, rat, etc.) and any amino acids that are variant between the two sequences may be substituted into the human PDZ domain to make a variant of the PDZ domain. In some embodiments, the PDZ domain polypeptide used to capture E6 protein in a sample is MAGI-1. For example, the sequence of the human MAGI-1 PDZ domain 2 may be compared to equivalent MAGI-1 PDZ domains from other species (e.g. mouse Genbank GI numbers 7513782 and 28526157 or other homologous sequences) to identify amino acids that may be substituted into the human MAGI-1-PDZ domain to make a variant thereof. Such method may be applied to any of the MAGI-1 PDZ domains described herein. Particular variants may have 1, up to 5, up to about 10, up to about 15, up to about 20 or up to about 30 or more, usually up to about 50 amino acid changes as compared to a sequence set forth in the sequence listing. In making a variant, if a GFG motif is present in a PDZ domain, in general, it should not be altered in sequence. Exemplary PDZ domain peptides are disclosed in U.S. Pat. Nos. 7,312,041 and 7,399,467, which are herein incorporated by reference in their entirety.

In general, variant PDZ domain polypeptides have a PDZ domain that has at least about 70 or 80%, usually at least about 90%, and more usually at least about 98% sequence identity with a variant PDZ domain polypeptide described herein, as measured by BLAST 2.0 using default parameters, over a region extending over the entire PDZ domain.

As used herein, the term “PDZ protein” refers to a naturally occurring protein containing a PDZ domain. Exemplary PDZ proteins include CASK, MPP1, DLG1, DLG2, PSD95, NeDLG, TIP-33, SYN1a, TIP-43, LDP, LIM, LIMK1, LIMK2, MPP2, NOS1, AF6, PTN-4, prIL16, 41.8 kD, KIAA0559, RGS12, KIAA0316, DVL1, TIP-40, TIAM1, MINT1, MAGI-1, MAGI-2, MAGI-3, KIAA0303, CBP, MINT3, TIP-2, KIAA0561, and TIP-1.

As used herein, the term “PL protein” or “PDZ Ligand protein” refers to a protein that forms a molecular complex with a PDZ-domain, or to a protein whose carboxy-terminus, when expressed separately from the full length protein (e.g., as a peptide fragment of 4-25 residues, e.g., 8, 10, 12, 14 or 16 residues), forms such a molecular complex. The molecular complex can be observed in vitro using a variety of assays described infra. As used herein, a “PL sequence” refers to the amino acid sequence of the C-terminus of a PL protein (e.g., the C-terminal 2, 3, 4, 5, 6, 7, 8, 9, 10, 12, 14, 16, 20 or 25 residues) (“C-terminal PL sequence”) or to an internal sequence known to bind a PDZ domain (“internal PL sequence”).

A phenotypic characteristic of all high-risk oncogenic HPV E6 proteins is the presence of a conventional PDZ binding motif (X-S/T-X-φ) where X is any amino acid and φ represents a hydrophobic amino acid. This PDZ domain binding motif is located at the last four carboxy-terminal (C-terminal) amino acids of oncogenic E6 proteins of high-risk HPV strains. In contrast, all low-risk HPV strains do not contain this PDZ domain binding motif. As used herein, the term “PDZ domain” refers to protein sequence of less than approximately 90 amino acids, (i.e., about 80-90, about 70-80, about 60-70 or about 50-60 amino acids), characterized by homology to the brain synaptic protein PSD-95, the Drosophila septate junction protein Discs-Large (DLG), and the epithelial tight junction protein ZO1 (Z01). PDZ domains are also known as Discs-Large homology repeats (“DHRs”) and GLGF repeats. PDZ domains generally appear to maintain a core consensus sequence (Doyle, D. A., 1996, Cell 85: 1067-76). PDZ domains are found in diverse membrane-associated proteins including members of the MAGUK family of guanylate kinase homologs, several protein phosphatases and kinases, neuronal nitric oxide synthase, tumor suppressor proteins, and several dystrophin-associated proteins, collectively known as syntrophins. Based upon PDZ ligand binding, all of the high-risk HPV types can be classified as Class I PDZ binding proteins. HPV E6 is known to interact with six different PDZ domain-containing proteins including but not limited to discs large (Dig), MAGI-1, MAGI-2, MAGI-3, MUPP1, and Scribble (hScrib). These PDZ domain-containing proteins are characterized by having multiple protein-protein interaction motifs and are frequently expressed at sites of cell to cell contact. They function mostly by regulating the formation of multicomponent protein complexes at these sites by interaction of their PDZ domains. Through the PDZ domain binding sequences, E6 protein can bind a single PDZ domain on each target protein and then direct its deregulation by the 26S proteosome. In cervical tumor models, it has been demonstrated that expression of oncogenic E6 proteins of high-risk HPV strains targets hDLg, MAGI-1 and MUPP1. All three of these proteins suppress E6-induced cell transformation, suggesting that in the context of HPV-induced transformation, hDLg, MAGI-1 and MUPP1 can function as tumor suppressors. Furthermore, in terms of the PDZ domain binding motif (X-S/T-X-φ) at the C-terminus of oncogenic E6 proteins, greater than 87% of the most prevalent high-risk HPV types necessary for progression into cervical cancer encode either E-T-Q-L (HPV16-like) or E-T-Q-V (HPV198-like). In some aspects, the present invention relates to the generation of specific antibodies, preferably monoclonal antibodies (mAbs), against amino acid sequences E-T-Q-L or E-T-Q-V for use as diagnostic or therapeutic immunoreagents for cancer, for example, cervical cancer.

As used herein, a “carboxy-terminal sequence” or “C-terminal sequence” refers to the amino acid sequence of the C-terminus of E6 protein, for example, the C-terminal 2, 3, 4, 5, 6, 7, 8, 9, 10, 12, 14, 16, or 25 residues of an oncogenic E6 protein. A comparison of E6 protein PDZ binding domains between high-risk and low-risk HPV strains is shown in Table 1.

TABLE 1

| | | Oncogenic | |
|---|---|---|---|
| HPV Type | E6 C-terminal Sequence | (X-S/T-X-V/L) | Cervical Cancer |
| 16 | SSRTRRETQL | Yes | 58% |
| 18 | RLQRRRETQV | Yes | 15% |
| 26 | RPRRQTETQV | Yes | |
| 30 | RRTLRRETQV | Yes | |
| 31 | WRRPRTETQV | Yes | 6% |
| 39 | RRLTRRETQV | Yes | |
| 45 | RLRRRRETQV | Yes | 9% |
| 51 | RLQRRNETQV | Yes | |
| 68 | RRRTRQETQV | Yes | |
| 69 | RRREATETQV | Yes | |
| 82 | PPRQRSETQV | Yes | |
| Other HR | ...............X-S/T-X-V/L | Yes | |
| 1A | KCSLCRLYAI | No | |
| 6b | WTTCMEDMLP | No | |
| 11 | WTTCMEDLLP | No | |

Antibody Compositions

The terms “antibody” and “immunoglobulin” are used interchangeably herein to refer to a type capture agent that has at least an epitope binding domain of an antibody. These terms are well understood by those in the field, and refer to a protein containing one or more polypeptides that specifically binds an antigen. One form of antibody constitutes the basic structural unit of an antibody. This form is a tetramer and consists of two identical pairs of antibody chains, each pair having one light and one heavy chain. In each pair, the light and heavy chain variable regions are together responsible for binding to an antigen, and the constant regions are responsible for the antibody effector functions.

The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

The terms “antibodies” and “immunoglobulin” include antibodies or immunoglobulins of any isotype (IgM, IgG, IgD, IgE, or IgA), fragments of antibodies which retain specific binding to antigen, including, but not limited to, Fab, Fv, scFv, and Fd fragments, chimeric antibodies, humanized antibodies, single-chain antibodies, peptides, peptidomimetics, peptoids, peptibodies, and fusion proteins comprising an antigen-binding portion of an antibody and a non-antibody protein. The antibody may be in any suitable form e.g., monoclonal, polyclonal, or synthetic. The antibodies may be detectably labeled, e.g., with a radioisotope, an enzyme which generates a detectable product, a fluorescent protein, and the like. The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the terms are Fab′, Fv, F(ab′)₂, and or other antibody fragments that retain specific binding to antigen.

Antibodies may exist in a variety of other forms including, for example, Fv, Fab, and F(ab′)₂, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al, Immunology, Benjamin, N.Y., 2nd ed. (1984), and Hunkapiller and Hood, Nature, 323, 15-16 (1986). Monoclonal antibodies, polyclonal antibodies, and “phage display” antibodies are well known in the art and encompassed by the term “antibodies”.

In one aspect, the invention provides an antibody composition, particularly monoclonal antibodies, which bind to the N-terminal end of E6 proteins of at least two strains of HPV, in some embodiments, at least two oncogenic HPV strains, or at least three oncogenic HPV strains, and in other embodiments, at least six oncogenic HPV strains. In other words, the invention provides antibodies that “recognize”, i.e., specifically bind to with K_{D} of 10⁻⁶M, 10⁻⁷ M, 10⁻⁸ M, 10⁻⁹ M, 10⁻¹⁰ M, 10⁻¹¹ M, 10⁻¹² M or less, multiple E6 proteins at their N-terminus. In other words, the subject antibodies each bind to (i.e., cross-react with) a plurality of different E6 proteins (i.e., at least 2, at least 3, at least 4, at least 5, at least 6 or at least 10, usually up to about 12, 15 or 20 or more different E6 proteins) from oncogenic strains of HPV. In general, the subject antibodies bind to amino acid motifs that are conserved between the oncogenic E6 proteins of different high-risk HPV strains, and, accordingly, bind to E6 proteins that have this motif. In some embodiments, the N-terminal amino acid sequence motif of oncognenic E6 proteins is HPV-16 like. One example of such amino acid sequence is F-Q-D-P-A-E-R-P-R-K-L-H-D-L-C-T-E-L. In other embodiments, the N-terminal amino acid sequence motif of oncognenic E6 proteins is HPV-18 like. One example of such amino acid sequence is F-Q-D-P-A-E-R-P-Y-K-L-P-D-L-C-T-E-L. In certain embodiments, the antibodies bind at least the E6 proteins of HPV strains 16 and 18 (e.g. the E6 of HPV strains 16, 18, 31, 33 and 45; 16, 18 and 45; or, in other embodiments, the E6 proteins of the HPV strains 16, 18, 26, 30, 31, 34, 39, 45, 51, 52, 53, 58, 59, 66, 68, 69, 70, 73, and 82). In other embodiments, the antibodies bind to at least the E6 proteins from HPV strains 16 and 45. In yet other embodiments, the antibodies bind to E6 proteins from two or more HPV strains including but not limited to HPV16, 18, 26, 30, 31, 34, 39, 45, 51, 52, 53, 58, 59, 66, 68, 69, 70, 73, 82, 6, 11, 40, 42, 43, 44, 54, 61, 72, and 81.

The subject antibodies may specifically bind to sequence motifs found in the N-terminal end of oncogenic HPV E6 proteins. E6 is a 151 amino-acid peptide that incorporates a type 1 motif with a consensus sequence -(T/S)-(X)-(V/I)-COOH. As used herein, an “amino-terminal sequence” or “N-terminal sequence” refers to the amino acid sequence of the N-terminus of E6 protein, for example, the N-terminal 2, 3, 4, 5, 6, 7, 8, 9, 10, 12, 14, 16, 20, 25, 30, 35, 40, 45, 50, 55, 60, 65, 70, or 75 residues of an oncogenic E6 protein.

In order to produce antibodies specific for the high risk group of HPV strains, synthetic peptide encoding a T cell epitope, for example, F-I-S-E-A-I-I-H-V-L-H-S-R, is fused with an oncogenic E6 N-terminal peptide disclosed herein. Not intended to be bound by any theory, having a T cell epitope in the immunogen may increase the immunogenecity of the immunogen and lead to a greater T cell and B cell response. The immunogen of the present invention, i.e. the T cell epitope fused with an oncogenic E6 N-terminal peptide, may result in an increased amount of antibody production upon immunization. The oncogenic E6 N-terminal peptide may contain the amino acid sequences F-Q-D-P-A-E-R-P-R-K-L-H-D-L-C-T-E-L or F-Q-D-P-A-E-R-P-Y-K-L-P-D-L-C-T-E-L. These fused peptides are used as the antigens to stimulate the production of antibodies that are specific against the N-terminus of oncogenic E6 proteins of at least two high-risk HPV strains. Many T cell epitopes capable of stimulating antibody production are known in the art and are within the scope of the present invention.

The antibodies of the present invention bind to E6 proteins with increased binding affinity and detect E6 proteins with increased sensitivity as compared to antibodies previously available. In some embodiments, the antibody binds to E6 protein from one or more HPV strains with a binding affinity of less than 10⁻⁸ M. In some embodiments, the antibody binds to E6 protein from one or more HPV strains with a binding affinity of less than 10⁻⁹ M. In some embodiments, the antibody binds to E6 protein from one or more HPV strains with a binding affinity of less than 10⁻¹⁰ M. In some embodiments, the antibody binds to E6 protein from one or more HPV strains with a binding affinity of less than 10⁻¹¹ M. In some embodiments, the antibody binds to E6 protein from one or more HPV strains with a binding affinity of less than 10⁻¹² M.

Examples of the purified monoclonal antibody specific for the N-terminal end of oncogenic E6 proteins of at least two HPV strains include but are not limited to hybridoma cell lines 4E9.7 (ATCC #PTA-9679), 4E910.2 (ATCC #PTA-9680), 6H5.3 (ATCC #PTA-9681), 2H9.15 (ATCC #PTA-9871), 7E7.7 (ATCC #PTA-9691), PAEP 8G83 (ATCC #PTA-9685), PAEP 3A10.25 (ATCC #PTA-9686), MMP7-5G11.9 (ATCC #PTA-9682), MMP7-15H8.12 (ATCC #PTA-9683), and PAEP 2G7.1 (ATCC #PTA-9684), which have been deposited at the American Type Culture Collection (ATCC). The subject antibodies can be used as capture antibodies, which may be immobilized to a substrate to capture E6 proteins in a sample, or can be used as detector antibodies, which bind to E6 protein that is bound to another E6 binding partner recognizing a different binding site on the E6 protein.

In one aspect, the invention provides an antibody composition, particularly monoclonal antibodies, which bind to the C-terminal end of oncogneic E6 proteins of at least two strains of HPV, in some embodiments, at least three oncogenic HPV strains, and in other embodiments, at least six oncogenic HPV strains. In other words, the invention provides antibodies that “recognize”, i.e., specifically bind to with K_{D} of 10⁻⁶ M, 10⁻⁷ M, 10⁻⁸ M, 10⁻⁹ M, 10⁻¹⁰ M, 10⁻¹¹ M, 10⁻¹²M, or less, multiple E6 proteins at their C-terminus. In other words, the subject antibodies each bind to (i.e., cross-react with) a plurality of different E6 proteins (i.e., at least 2, at least 3, at least 4, at least 5, at least 6 or at least 10, usually up to about 12, 15 or 20 or more different E6 proteins) from oncogenic strains of HPV. In general, the subject antibodies bind to amino acid motifs that are conserved between the oncogenic E6 proteins of different high-risk HPV strains, and, accordingly, bind to E6 proteins that have this motif. In some embodiments, the C-terminal amino acid sequence motif of oncognenic E6 proteins is E-(T/S)-X-(V/L). The motif may be the HPV16 E6-like sequence E-T-Q-L or the HPV18 E6-like sequence E-T-Q-V. In certain embodiments, the antibodies bind at least the E6 proteins of HPV strains 16 and 18 (e.g. the E6 of HPV strains 16, 18, 31, 33 and 45; 16, 18 and 45; or, in other embodiments, the E6 proteins of the HPV strains 16, 18, 26, 30, 31, 34, 39, 45, 51, 52, 53, 58, 59, 66, 68, 69, 70, 73, and 82). In other embodiments, the antibodies bind to at least the E6 proteins from HPV strains 16 and 45.

The subject antibodies may specifically bind to sequence motifs found in the C-terminal end of oncogenic HPV E6 proteins. In order to produce antibodies specific for the high risk group of HPV strains, synthetic peptide encoding a T cell epitope, for example, F-I-S-E-A-I-I-H-V-L-H-S-R, is fused with an oncogenic E6 protein PDZ binding motif. Not intended to be bound by any theory, having a T cell epitope in the immunogen may increase the immunogenecity of the immunogen and lead to a greater T cell and B cell response. The immunogen of the present invention, i.e. the T cell epitope fused with an oncogenic E6 protein PDZ binding motif, may result in an increased amount of antibody production upon immunization. The oncogenic E6 protein PDZ binding motif may contain the amino acid sequence E-(T/S)-X-(V/L), for example, E-T-Q-L or E-T-Q-V. These fused peptides are used as the antigens to stimulate the production of antibodies that are specific against the C-terminus of oncogenic E6 proteins of at least two high-risk BIN strains. The chimeric peptide sequences consisting of a T cell epitope fused with the C-terminal PDZ binding motif of oncogenic E6 proteins are shown in Table 2.

TABLE 2
Immunizing peptide:
F-I-S-E-A-I-I-H-V-L-H-S-R-R-R-E-T-Q-L
F-I-S-E-A-I-I-H-V-L-H-S-R-C-R-Q-C-W-R-R-R-R-R-E-T-Q-L
Screening peptides:
F-I-S-E-A-I-I-H-V-L-H-S-R-C-R-Q-C-W-R-R-R-R-R-E-T-Q-L
F-I-S-E-A-I-I-H-V-L-H-S-R-C-R-Q-C W-R-R-R
R-R-E-T-Q-L
Immunizing peptide:
F-I-S-E-A-I-I-H-V-L-H-S-R- R-R-E-T-Q-V
F-I-S-E-A-I-I-H-V-L-H-S-R-C-R-Q-C-W-R-R-R-R-R-E-T-Q-V
Screening peptides:
F-I-S-E-A-I-I-H-V-L-H-S-R-C-R-Q-C-W-R-R-R-R-R-E-T-Q-V
F-I-S-E-A-I-I-H-V-L-H-S-R-C-R-Q-C-W-R-R-R
R-R-E-T-Q-V

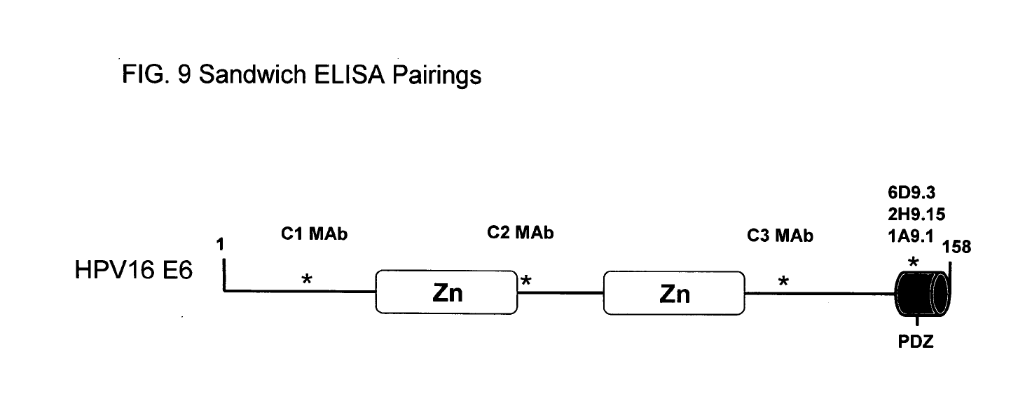

In one example, the purified monoclonal antibody specific for the C-terminal end of oncogenic E6 proteins of at least two MN strains is 6D9.3. In another example, the purified monoclonal antibody specific for the C-terminal end of oncogenic E6 proteins of at least two HPV strains is 1A9.1. In another example, the purified monoclonal antibody specific for the C-terminal end of oncogenic E6 proteins of at least two HPV strains is 2H9.15. These antibodies can be used as capture antibodies, which may be immobilized to a substrate to capture E6 proteins in a sample, or can be used as detector antibodies, which bind to E6 protein that is bound to another capture antibody recognizing a different binding site on the E6 protein. As shown in FIG. 9, various anti-HPV E6 capture antibodies were used to develop sandwich ELISA assays to detect HPV16 E6 protein. Combining antibody libraries (capture C1, C2, and C3 MAb) with the HPV oncopeptide PDZ antibodies identified the capture/detector pairs.

In one aspect, the present invention provides an antibody composition for detecting E6 protein of at least one HPV strain in a sample, the composition comprising a first antibody and a second antibody, wherein the first antibody is immobilized and binds to E6 protein of an oncogenic HPV strain in the sample, and the second antibody specifically binds to the E6 protein that is bound by the first antibody, and wherein the second antibody is part of a signal producing system for detection of the E6 protein in the sample. In another aspect, the present invention provides a method of detecting E6 protein of at least one strain of HPV in a sample, comprising (a) contacting an immobilized first antibody which specifically binds to E6 protein of at least one strain of HPV with the sample, (b) contacting the E6 protein that is bound to the immobilized first antibody with a second antibody, which specifically binds to E6 protein of at least one strain of HPV; and (c) detecting binding of the second antibody to the E6 protein, thereby detecting the E6 protein in the sample; wherein binding of the second antibody to the E6 protein indicates the presence of at least one HPV strain in the sample.

In some embodiments, detection of more than 1 oncogenic E6 protein (e.g., all oncogenic E6 proteins, E6 proteins from HPV strains 16 and 18, or E6 proteins from HPV strains 16 and 45 etc.) is desirable, and, as such, an oncogenic E6 protein binding partner may be antibody that binds to these proteins, as described below, or a mixture of antibodies that each bind to a different proteins. As is known in the art, such binding partners may be labeled to facilitate their detection. In general, an antibody binds E6 with an binding affinity of less then 10⁻⁵ M, e.g., less than 10⁻⁶, less than 10⁻⁷, less than 10⁻⁸ M (e.g., less than 10⁻⁹ M, 10⁻¹⁰, 10⁻¹¹, etc.).

In one example, a PDZ domain polypeptide may be compared to antibodies specific against E6 proteins of at least one oncogenic HPV strain described herein in terms of their binding affinity, specificity, sensitivity for binding to E6 protein and the false positive rate of detecting E6, which refers to the rate of erroneously detecting an E6 protein of an oncogenic HPV strain when such E6 protein or HPV strain is in fact absent in a sample. In some embodiments, the method of the present invention enhances signal-to-noise ratio of detecting an oncogenic E6 protein as compared to using a PDZ domain containing polypeptide for the detection of E6 protein in a sample. The signal-to-noise ratio of detecting an oncogenic E6 protein using the composition and method of the present invention may be increased by about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 fold or more, as compared to using a PDZ domain polypeptide to detect the E6 protein. In some embodiments, the method has higher specificity of detecting an oncogenic HPV E6 protein as compared to using a PDZ domain containing polypeptide to detect the E6 protein. The method may also have higher sensitivity of detecting an oncogenic HPV E6 protein as compared to using a PDZ domain containing polypeptide to detect the E6 protein. In other embodiments, the method results in a lower false positive rate of erroneously detecting an oncogenic HPV E6 protein as compared to using a PDZ domain containing polypeptide to detect the E6 protein. The false positive rate of detecting E6 protein of an oncogenic HPV strain in a sample using the composition and method of the present invention may be less than 10%, 9%, 8%, 7, %, 6%, 5%, 4.5%, 4%, 3.5%, 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1% or less.

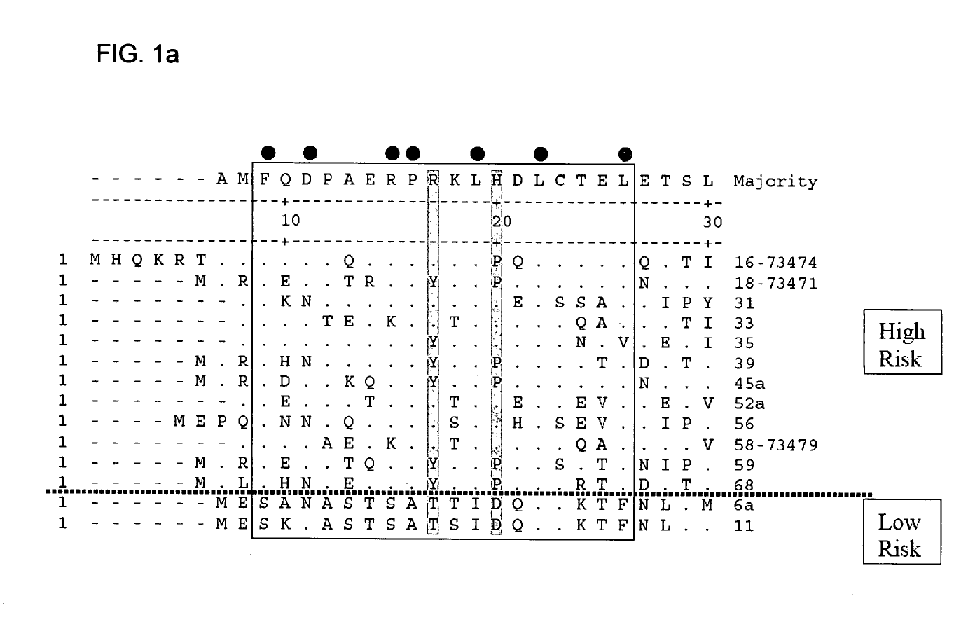

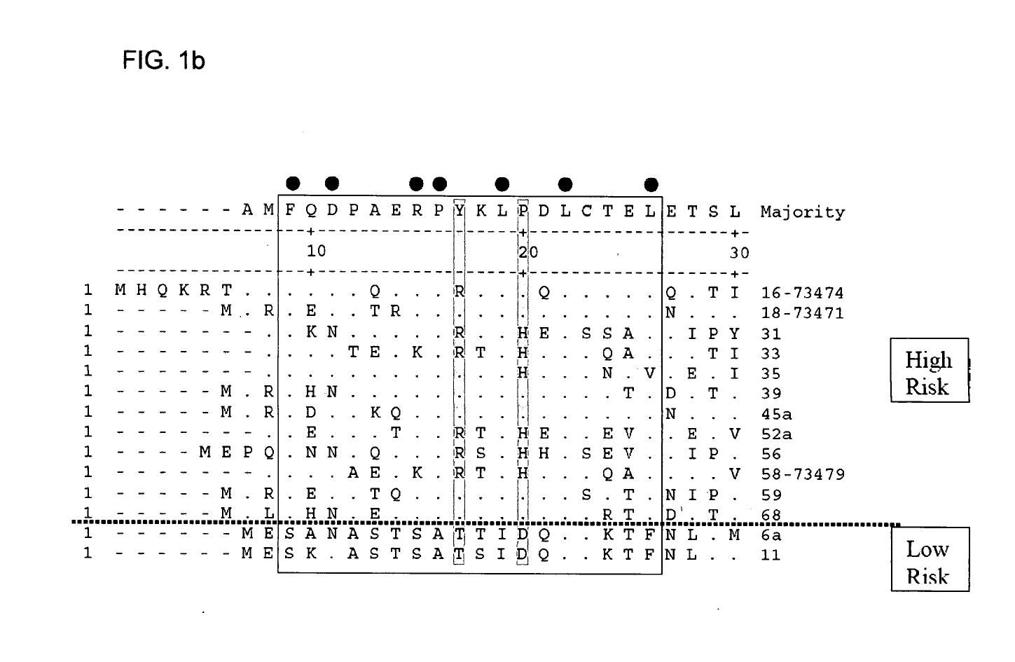

The subject antibodies may bind to any portion or region of HPV E6 proteins. In some embodiments, the subject antibodies may bind to one of three sequence motifs found in HPV E6 proteins. These motifs are disclosed in details in FIG. 1 of U.S. Pat. No. 7,399,467, which is herein incorporated by reference in its entirety, and generally correspond to regions of sequence similarity between E6 proteins from different strains of HPV. In general, therefore, a subject antibody binds to peptides having the following sequence: FQDPQERPRKLPQLCTELQTTIHDI (SEQ ID NO:1) and FEDPTRRPYKLPDLCTELNTSLQDI (SEQ ID NO:2), corresponding to a first common sequence motif in the E6 proteins of HPV strains 16 and 18, respectively, LLIRCINCQKPLCPEEKQRHLDK (SEQ ID NO:3) and LLIRCLRCQKPLNPAEKLRHLNE (SEQ ID NO:4), corresponding to a second common sequence motif in the E6 proteins of HPV strains 16 and 18, respectively, or RHLDKKQRFHNIRGRWTGRCMSCC (SEQ ID NO:5) and RHLNEKRRFHNIAGHYRGQCHSCC (SEQ ID NO:6) corresponding to a third common sequence motif in the E6 proteins of HPV strains 16 and 18, respectively. If a subject antibody binds to other E6 proteins, then it usually binds to the other E6 proteins at positions equivalent to those discussed above, or disclosed in U.S. Pat. No. 7,399,467, where “positions equivalent to” generally means a stretch of contiguous amino acids that correspond to, i.e., are aligned with, the boxed amino acids when the sequence of the other E6 proteins are with those in FIG. 1 of U.S. Pat. No. 7,399,467.

Accordingly, since antibodies generally recognize motifs smaller than those listed above, a subject antibody may recognize peptides that are smaller than and contained within the motifs described above. For example, a subject antibody may bind to a peptide having any 9 contiguous amino acids set forth in any one of SEQ NOS:1-6. In particular, a subject antibody may recognize the sequences RPRKLPQLCTEL (SEQ ID NO:7) and RPYKLPDLCTEL (SEQ ID NO:8), corresponding to sub-sequences of the first common sequences of E6 proteins of HPV strains 16 and 18, described above, LLIRCINCQKPL (SEQ ID NO:9) and LLIRCLRCQKPL (SEQ ID NO:10) corresponding to sub-sequences of the second common sequences of E6 proteins of HPV strains 16 and 18, as described above, or RHLDKKQRFHNI (SEQ ID NO:11) and RHLNEKRRFHNI (SEQ ID NO:12) corresponding to sub-sequences of the third common sequences of E6 proteins of HPV strains 16 and 18, as described above. Since these sub-sequences are generally conserved between different E6 proteins, as discussed above, antibodies that bind to the above-recited sequences generally bind to E6 proteins from other HPV strains.

In certain alternative embodiments, the subject antibodies will bind to E6 proteins from HPV strains 16, and 45. In general, therefore, a subject antibody binds to peptides having the following sequence: FQDPQERPRKLPQLCTELQTTIHDI (SEQ ID NO:1) and FDDPKQRPYKLPDLCTELNTSLQDV (SEQ ID NO:57), corresponding to a first common sequence motif in the E6 proteins of HPV strains 16 and 45, respectively, LLIRCINCQKPLCPEEKQRHLDK (SEQ ID NO:3) and LLIRCLRCQKPLNPAEKRRHLKD (SEQ ID NO: 58), corresponding to a second common sequence motif in the E6 proteins of HPV strains 16 and 45, respectively, or RHLDKKQRFHNIRGRWTGRCMSCC (SEQ ID NO:5) and RHLKDKRRFHSIAGQYRGQCNTCC (SEQ ID NO:59) corresponding to a third common sequence motif in the E6 proteins of HPV strains 16 and 45, respectively. If a subject antibody binds to other E6 proteins, then it usually binds to the other E6 proteins at positions equivalent to those discussed above.

Accordingly, since antibodies generally recognize motifs smaller than those listed above, a subject antibody may recognize peptides that are smaller than and contained within the motifs described above. For example, a subject antibody may bind to a peptide having any 9 contiguous amino acids set forth in any one of SEQ NOS:1, 3, 5, 57, 58 and 59. In particular, a subject antibody may recognize the sequences RPRKLPQLCTEL (SEQ ID NO:7) and RPYKLPDLCTEL (SEQ ID NO:60), corresponding to sub-sequences of the first common sequences of E6 proteins of HPV strains 16 and 45, described above, LLIRCINCQKPL (SEQ ID NO:9) and LLIRCLRCQKPL (SEQ ID NO: 61) corresponding to sub-sequences of the second common sequences of E6 proteins of HPV strains 16 and 45; as described above, or RHLDKKQRFHNI (SEQ ID NO:11) and RHLKDKRRFHSI (SEQ ID NO: 62) corresponding to sub-sequences of the third common sequences of E6 proteins of HPV strains 16 and 45, as described above. Since these sub-sequences are generally conserved between different E6 proteins, as discussed above, antibodies that bind to the above-recited sequences generally bind to E6 proteins from other HPV strains. In certain embodiments, cysteine residues can be replaced by serine residues to avoid disulfide bridge formation.

In some embodiments, the subject antibodies may bind to the carboxy or C-terminus of a HPV protein, for example, the C-terminus of E6 protein. Antibodies specific for the HPV C-terminal PDZ ligand (PL) motif may be used for both capture and detection of E6 protein of at least one oncogenic HPV strain, and for the treatment of HPV infection. The first antibody may bind to N-terminus of E6 protein and the second antibody may bind to C-terminus of the E6 protein. Alternatively, the first antibody may bind to C-terminus of E6 protein and the second antibody may bind to N-terminus of the E6 protein.

In some embodiments, the antibodies of the present invention bind to E6 protein of at least one oncogenic HPV strain selected from the group consisting of HPV strains 16, 18, 26, 30, 31, 33, 35, 39, 45, 51, 52, 53, 56, 58, 59, 66, 68, 69, 73, and 82. The subject antibodies may bind to E6 proteins of at least 1, 2, 3, 4, 5, 6, 7, 8, 9, 10, 11, 12, 13, 14, 15, or more high-risk HPV strains. In some embodiments, the first and/or second antibody bind to E6 of HPV 16, 18, 31, 33, 45, 52 or 58. The antibody composition of the present invention may specifically bind to E6 proteins of more than one HPV strain, thereby detecting the presence of more than one oncogenic HPV strain in a sample. The oncogenic HPV strains in a sample may be HPV strains 16, 18, 31, 33, 45, 52, 58 or a combination thereof. In other embodiments, the antibodies of the present invention bind to E6 proteins of low-risk HPV strains, for example, HPV6 and HPV11. There is a risk of perinatal infection of the fetus in pregnant women with low risk HPV types resulting in diseases such as respiratory papillomatosis.

In some embodiments, the second antibody is labeled for detection of E6 protein bound to the second antibody. The second antibody may be conjugated to an enzyme, such as horseradish peroxidase. In some embodiments, the antibody composition further comprises a third antibody which binds to the second antibody that is bound to E6 protein for detection of the E6 protein. The third antibody may be conjugated to a molecule that produces a signal, which indicates binding of the antibodies to E6 protein in the sample. In one example, the third antibody is conjugated to an enzyme, such as horseradish peroxidase. The presence of E6 that is bound by the first and the second antibodies may be detected using various techniques well known in the art. In some embodiments, the detection of bound E6 protein is by a sandwich enzyme-linked immunosorbent assay (ELISA), which is described in great details infra. The antibody composition of the present invention may be used as a part of a test for cervical cancer.

In some embodiments, the antibody composition enhances signal-to-noise ratio of detecting an oncogenic E6 protein as compared to using a PDZ domain containing polypeptide for the detection of an oncogenic E6 protein in a sample. The antibody composition of the present invention can be used to detect oncogenic E6 protein in a sample with a low false positive rate. The false positive rate of detecting E6 protein of an oncogenic HPV strain in a sample using the composition and method of the present invention may be less than 10%, 9%, 8%, 7, %, 6%, 5%, 4.5%, 4%, 3.5%, 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1% or less. In one example, the false positive rate is less than 1.7%.

E6 Binding Partner

In one aspect, the subject antibody is used as a capture antibody to capture E6 protein in an enzyme-linked immunosorbent assay (ELISA). In some embodiments, the method is an enzyme-linked immunosorbent assay (ELISA), comprising: (a) contacting the subject antibody with the sample; (b) contacting the E6 protein that is bound to the subject antibody with another E6 binding partner that specifically binds to the E6 protein at a binding site that is different from that of the subject antibody; and (c) detecting binding of the E6 binding partner to the E6 protein, thereby detecting the presence of the E6 protein in the sample. In another aspect, the antibody is used as a detector antibody to detect E6 protein that is bound to an immobilized E6 binding partner specific for the E6 protein in an ELISA. In some embodiments, the method is an ELISA comprising: (a) contacting the sample with an E6 binding partner that specifically binds to E6 protein at a binding site that is different from that of the subject antibody; (b) contacting the E6 protein that is bound to the E6 binding partner with the subject antibody; and (c) detecting binding of the subject antibody to the E6 protein, thereby detecting the presence of the E6 protein in the sample.

An “E6 protein binding partner” can be any molecule that specifically binds to an oncogenic E6 protein. Suitable oncogenic E6 protein binding partners include a PDZ domain (as described below), antibodies against oncogenic E6 proteins (such as those described below); other proteins that recognize oncogenic E6 protein (e.g., p53, E6-AP or E6-BP); DNA (i.e., cruciform DNA); and other partners such as aptamers. In some embodiments, detection of more than 1 oncogenic E6 protein (e.g., all oncogenic E6 proteins, E6 proteins from HPV strains 16 and 18, or E6 proteins from HPV strains 16 and 45 etc.) is desirable, and, as such, an oncogenic E6 protein binding partner may be antibody that binds to these proteins, as described below, or a mixture of antibodies that each bind to a different proteins. As is known in the art, such binding partners may be labeled to facilitate their detection. In general, binding partners bind E6 with an binding affinity of less then 10⁻⁵ M, e.g., less than 10⁻⁶, less than 10⁻⁷, less than 10⁻⁸ M (e.g., less than 10⁻⁹ M, 10⁻¹⁰, 10⁻¹¹, 10⁻¹² etc.).

In some embodiments, the E6 binding partner used in the method of the present invention may include, but are not be limited to, p53, E6-AP, E6-BP or engineered compounds that bind E6 oncoproteins. Alternatively, one could also use DNA binding or Zn²⁺ binding to assay for the presence of captured E6 protein, since oncogenic E6 proteins are known to bind certain DNA structures through the use of divalent cations. Additionally, one could use the PDZ-captured E6 protein in an activity assay, since E6 is known to degrade DNA and certain proteins including p53 in the presence of a reticulocyte lysate. In most embodiments, the first E6 binding partner that captures E6 protein in a sample binds to the E6 protein at a location on the E6 protein that does not reduce the availability of the E6 protein for binding to the subject antibody pair.

In some embodiments, the E6 peptide used in the immunization of the animal contains a carrier, for example, Keyhole Limpet Hemocyanin (KLH), which is widely used as a carrier protein in antibody production (E6-KLH), or E6 fused to ovalbumin (E6-OVA). The E6 peptide or the relevant portion may be synthesized using conventional biochemistry methodology well known in the art. For example, the peptides may be prepared in linear form using conventional solution or solid phase peptide syntheses and cleaved from the resin followed by purification procedures (Creighton, 1983, Protein Structures And Molecular Principles, W. H. Freeman and Co., N.Y.). Suitable procedures for synthesizing the peptides described herein are well known in the art. The composition of the synthetic peptides may be confirmed by amino acid analysis or sequencing (e.g., the Edman degradation procedure and mass spectroscopy). In some embodiments, synthetic peptides of defined sequence (e.g., corresponding to the amino-terminus of E6 proteins) can be synthesized by any standard resin-based method (see, e.g., U.S. Pat. No. 4,108,846; see also, Caruthers et al., 1980, Nucleic Acids Res. Symp. Ser., 215-223; Horn et al., 1980, Nucleic Acids Res. Symp. Ser., 225-232; Roberge, et al., 1995, Science 269:202). The peptides used in the assays described herein can be prepared by the FMOC (see, e.g., Guy and Fields, 1997, Meth. Enz. 289:67-83; Wellings and Atherton, 1997, Meth. Enz. 289:44-67). In some cases (e.g., for use in the A and G assays of the invention), peptides are labeled with biotin at the amino-terminus by reaction with a four-fold excess of biotin methyl ester in dimethylsulfoxide with a catalytic amount of base. The peptides are cleaved from the resin using a halide containing acid (e.g. trifluoroacetic acid) in the presence of appropriate antioxidants (e.g. ethanedithiol) and excess solvent lyophilized.

Following lyophilization, peptides can be redissolved and purified by reverse phase high performance liquid chromatography (HPLC). One appropriate HPLC solvent system involves a Vydac C-18 semi-preparative column running at 5 mL per minute with increasing quantities of acetonitrile plus 0.1% trifluoroacetic acid in a base solvent of water plus 0.1% trifluoroacetic acid. After HPLC purification, the identities of the peptides are confirmed by MALDI cation-mode mass spectrometry.

In addition, analogues and derivatives of the immunizing peptides can be chemically synthesized. The linkage between each amino acid of the peptides of the invention may be an amide, a substituted amide or an isostere of amide. Nonclassical amino acids or chemical amino acid analogues can be introduced as a substitution or addition into the sequence. Non-classical amino acids include, but are not limited to, the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Nα-methyl amino acids, and amino acid analogues in general. Furthermore, the amino acid can be D (dextrorotary) or L (levorotary).

The E6 immunizing peptide used in the method of the present invention can also be synthesized using conventional recombinant genetic engineering techniques. For recombinant production, a polynucleotide sequence encoding a linear form of the peptide is inserted into an appropriate expression vehicle, i.e., a vector which contains the necessary elements for the transcription and translation of the inserted coding sequence, or in the case of an RNA viral vector, the necessary elements for replication and translation. The expression vehicle is then transfected into a suitable target cell which will express the peptide. Depending on the expression system used, the expressed peptide is then isolated by procedures well-established in the art. Methods for recombinant protein and peptide production are well known in the art (see, e.g., Maniatis et al., 1989, Molecular Cloning: A Laboratory Manual, Cold Spring Harbor Laboratory, N.Y.; and Ausubel et al., 1989, Current Protocols in Molecular Biology, Greene Publishing Associates and Wiley Interscience, N.Y.). PCR products containing E6 protein or a portion thereof are subcloned into an expression vector to permit expression of fusion proteins containing the E6 protein or a portion thereof and a heterologous domain (i.e., a KLH or OVA).

A variety of host-expression vector systems may be utilized to express the recombinant E6 peptides described herein. These include, but are not limited to, microorganisms such as bacteria transformed with recombinant bacteriophage DNA or plasmid DNA expression vectors containing an appropriate coding sequence; yeast or filamentous fungi transformed with recombinant yeast or fungi expression vectors containing an appropriate coding sequence; insect cell systems infected with recombinant virus expression vectors (e.g., baculovirus) containing an appropriate coding sequence; plant cell systems infected with recombinant virus expression vectors (e.g., cauliflower mosaic virus or tobacco mosaic virus) or transformed with recombinant plasmid expression vectors (e.g., Ti plasmid) containing an appropriate coding sequence; or animal cell systems.

Methods of Generating E6 Specific Antibodies

In one aspect, the present invention provides a method of generating an antibody that binds to amino-terminus (N-terminus) of E6 proteins of at least two HPV strains in a sample, the method comprising: (a) immunizing animal with a T cell epitope sequence fused with an N-terminal sequence of oncognenic E6 protein; (b) obtaining B lymphocytes from the immunized animal; (c) fusing the B lymphocytes obtained from the immunized animal with myeloma cells to generate hybridoma cells secreting antibodies; and (d) screening the hybridoma cells for antibodies that specifically bind to the N-terminus of E6 proteins of at least two HPV strains.

In one aspect, the present invention provides a method of generating an antibody that binds to carboxyl-terminus (C-terminus) of oncognenic E6 proteins of at least two HPV strains in a sample, the method comprising: (a) immunizing animal with a T cell epitope sequence fused with a C-terminal sequence of oncognenic E6 protein; (b) obtaining B lymphocytes from the immunized animal; (c) fusing the B lymphocytes obtained from the immunized animal with myeloma cells to generate hybridoma cells secreting antibodies; and (d) screening the hybridoma cells for antibodies that specifically bind to the C-terminus of oncognenic E6 proteins of at least two oncogenic HPV strains.

For the production of antibodies, various host animals, including but not limited to rabbits, mice, rats, etc., may be immunized by injection with a peptide. The peptide may be attached to a suitable carrier, such as BSA or KLH, by means of a side chain functional group or linkers attached to a side chain functional group. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacilli Calmette-Guerin) and Corynebacterium parvum.

Monoclonal antibodies to a peptide may be prepared using any technique that provides for the production of antibody molecules by continuous cell lines in culture. These include but are not limited to the hybridoma technique originally described by Koehler and Milstein, 1975, Nature 256:495-497, the human B-cell hybridoma technique, Kosbor et al., 1983, Immunology Today 4:72; Cote et al., 1983, Proc. Natl. Acad. Sci. U.S.A. 80:2026-2030 and the EBV-hybridoma technique (Cole et al., 1985, Monoclonal Antibodies and Cancer Therapy, Alan R. Liss, Inc., pp. 77-96 (1985)). In addition, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce peptide-specific single chain antibodies.

Antibody fragments containing deletions of specific binding sites may be generated by known techniques. For example, such fragments include but are not limited to F(ab′)₂ fragments, which can be produced by pepsin digestion of the antibody molecule and Fab fragments, which can be generated by reducing the disulfide bridges of the F(ab′)₂ fragments. Alternatively, Fab expression libraries may be constructed (Huse et al., 1989, Science 246:1275-1281) to allow rapid and easy identification of monoclonal Fab fragments with the desired specificity for the peptide of interest.

The antibody or antibody fragment specific for the desired peptide can be attached, for example, to agarose, and the antibody-agarose complex is used in immunochromatography to purify peptides of the invention. See, Scopes, 1984, Protein Purification: Principles and Practice, Springer-Verlag New York, Inc., NY, Livingstone, 1974, Methods Enzymology: Immunoaffinity Chromatography of Proteins 34:723-731. Antibodies can also be linked to other solid supports for diagnostic applications, or alternatively labeled with a means of detection such an enzyme that can cleave a colorimetric substrate, a fluorophore, a magnetic particle, or other measurable compositions of matter.

In conjunction with the methods describe supra, one could employ a number of techniques to increase the likelihood of producing or selecting high affinity antibodies. An example is to prepare the E6 antigen (to raise antibodies against) in the same manner that one would prepare tissue or cell samples for testing. Alternatively, one could immunize with E6 fusion protein prepared in one manner, and screen for specific E6 antibodies using a second E6 protein prepared in a different manner. This should select for antibodies that recognize E6 epitopes that are conserved under different sample collection and preparation procedures. In another example, one could immunize animals with E6 antigen that has been rapidly denatured and renatured, such that epitopes that are insensitive to preparation conditions are selected for. Another method that could be employed is to use peptides corresponding to antigenic regions of the E6 proteins as predicted by Major Histocompatibility Complex (MHC) and T Cell Receptor (TCR) consensus binding.

In some embodiments, the antigen is fused to a T cell epitope, for example, a T cell epitope having an amino acid sequence F-I-S-E-A-I-I-H-V-L-H-S-R. In some embodiments, the C-terminal sequence of an oncognenic E6 protein used in the immunizing step contains a PDZ domain binding motif, for example, a conserved amino acid motif E-(T/S)-X-(V/L). The conserved amino acid motif may be E-T-Q-L or E-T-Q-V. Examples of immunizing peptides include but are not limited to peptides F-I-S-E-A-I-I-H-V-L-H-S-R-C-R-Q-C-W-R-R-R-R-R-E-T-Q-L and F-I-S-E-A-I-I-H-V-L-H-S-R-C-R-Q-C-W-R-R-R-R-R-E-T-Q-V, as shown in Table 2.

Upon synthesis of the chimeric peptide consisting of a T cell epitope fused with the HPV oncogenic PDZ consensus binding motif, intrasplenic immunizations are carried out in female Balb/c mice to stimulate the production of antibodies of the present invention. Accordingly, peptides having 9, 10, 11, 12, 13, 14, 15 or more, usually up to about 20 or more contiguous amino acids of any of the peptides described above may be used for immunizations. In some embodiments, a recited peptide sequence may be contained within a larger peptide that may be 1, 2, 3, 4, 5, 6, 7, 8, 9 or 10 or more, sometimes up to about 15 or 20 or more amino acids greater in size than a recited polypeptide. Accordingly, a subject peptide may be from about 8 to about 30 amino acids in length. In certain embodiments, a subject peptide is about 9-20 amino acids in length, and usually contains an amino acid sequence described above.

In order to produce antibodies specific for the high risk group of HPV, synthetic peptides with artificial sequences related to several high risk groups are designed as antigens. The peptide sequences used as the immunogens to generate the antibodies of the present invention are designed from a consensus of conserved amino acids found in the top high-risk HPV strains. In contrast, low-risk HPV strains retain very little homology to the consensus peptide sequence. The design of suitable peptides used in the immunization of animals for the production of HPV E6 antibodies of the present invention is shown in FIG. 1. The design is based on the premise that the resulting diagnostic should have a very high sensitivity to avoid false-negatives while maximizing specificity. To that end, a high degree of homology within oncogenic strains and low homology to low-risk HPV strains is the goal. Location at the N-terminus of E6 protein is desired. Difference to sequences encumbered by IP (i.e. HPV33) is intended. In addition, secondary structure prediction is not unambiguous, but the distribution of identical or similar residues allows for the possibility that they face the same side of the protein in an alpha-helical conformation which would be promising for generating a cross-reactive antibody, i.e. an antibody that binds to E6 proteins of at least two HPV strains.

In some embodiments, the peptide(s) used are KLH- and ovalbumin conjugates as well as free peptides. In addition, a multiple antigenic peptide (MAP)-based strategy is pursued in which the following T-cell epitope, FISEAIIHVLHSR, precedes the E6-peptide sequences disclosed herein.

Multiple antigenic peptide system (MAPS) is a method for producing high-titer anti-peptide antibodies (1,2) and synthetic peptide vaccines (3). This system utilizes the a- and c-amino functional groups of lysine to form a backbone to which multiple peptide chains are attached. Depending on the number of lysine tiers (2, 4, 8, etc.), different numbers of peptide branches can be synthesized. Using this new technology, high-titer antibodies can be produced (Posnett, D. et al. J. Biol. Chem. 263, 1719-1725 1988; Tam, J. P. PNAS USA 85, 5409-5413, 1988). Immunization with chemically defined synthetic polymers, multiple antigenic peptide (MAP) systems, containing T and B cell epitopes of various proteins induce high levels of circulating antibodies that are detectable several months after boosting. The anti-MAP secondary antibody response is characterized by an increase in the levels of circulating IgG and a concomitant decrease in the IgM levels. In vitro and in vivo experiments indicated that Th epitopes included in the MAP are recognized by T cells induced after immunization with the native protein and, also, that MAP-induced T cells can recognize the native protein. In addition to high levels of anti-B epitope antibodies, MAP immunization also induces antibodies against the T epitope. This anti-T epitope immune response does not affect the generation of the anti-B epitope antibodies. Immunization of different strains of mice revealed that the antibody response is consistent with the genetically restricted pattern of recognition of the T epitope. The findings of this study indicate that MAP are potent immunogens capable of inducing immunologic memory and are, thus, good candidates for the development of subunit vaccines designed to induce high levels of circulating antibodies. Since cysteines may pose potential problems for the MAP approach, a shorter version of MAP without the Cys residue may be considered, as an alternative a modified sequence with a Ser instead of Cys as in HPV strains 31 and 56 could also be used.

In some embodiments, a selective group of E6 proteins including E6 proteins from both high-risk and low-risk HPV strains is aligned to deduce a consensus peptide sequence that is based on the E6 peptide sequence of HPV16. One example of the consensus peptide sequence is F-Q-D-P-A-E-R-P-R-K-L-H-D-L-C-T-E-L. A list of the number of amino acids not matching the consensus peptide sequence F-Q-D-P-A-E-R-P-R-K-L-H-D-L-C-T-E-L within each HPV E6 protein sequence is shown in Table 3. In other embodiments, a selective group of E6 proteins including E6 proteins from both high-risk and low-risk HPV strains is aligned to deduce a consensus peptide sequence that is based on the E6 peptide sequence of HPV18. One example of the consensus peptide sequence is F-Q-D-P-A-E-R-P-Y-K-L-P-D-L-C-T-E-L. A list of the number of amino acids not matching the consensus peptide F-Q-D-P-A-E-R-P-Y-K-L-P-D-L-C-T-E-L within each HPV E6 protein sequence is shown in Table 4. In some embodiments, the peptide consensus sequence is then fused with the T cell epitope sequence disclosed hereinabove and the fusion peptide is used as an immunogen to stimulate the antibody production in the method of the present invention.

TABLE 3

| HPV strain | No. of unmatched AAs |
|---|---|
| 16 | 3 |
| 18 | 5 |
| 31 | 6 |
| 33 | 6 |
| 35 | 3 |
| 39 | 5 |
| 45 | 5 |
| 52 | 6 |
| 56 | 8 |
| 58 | 6 |
| 59 | 7 |
| 68 | 6 |
| 6b | 16 |
| 11 | 15 |

TABLE 4

| HPV strain | No. of unmatched AAs |
|---|---|
| 16 | 3 |
| 18 | 3 |
| 31 | 8 |
| 33 | 8 |
| 35 | 3 |
| 39 | 3 |
| 45 | 3 |
| 52 | 8 |
| 56 | 10 |
| 58 | 8 |
| 59 | 5 |
| 68 | 5 |
| 6b | 16 |
| 11 | 15 |

In some embodiments, exact matches of E6 from HPV-16 and HPV-18 are synthesized either as peptides or as recombinant proteins, and used as screening reagents since these two HPV strains are typically included in a detection assay before the cross-strain specificity of an antibody can be approached. In some embodiments, the consensus peptide can be generated based on any HPV E6 protein and the subject antibodies can be screened using any E6 protein.

Upon synthesis of the fusion peptide comprising a T cell epitope fused with the N-terminal consensus sequence of HPV E6 protein, in some embodiments, the E6 fusion peptide/protein is conjugated to KLH or ovalbumin, immunizations are carried out in animals to generate an immune response specific to epitopes within the consensus peptide. The immunization can be carried out in mice, for example, female Balb/c mice, via repetitive immunization multiple sites (RIMMS) immunization strategy, conventional immunization, or intrasplenic means to stimulate the production of antibodies of the present invention. Accordingly, peptides having 9, 10, 11, 12, 13, 14, 15 or more, usually up to about 20 or more contiguous amino acids of any of the peptides described above may be used for immunizations. In some embodiments, a recited peptide sequence may be contained within a larger peptide that may be 1, 2, 3, 4, 5, 6, 7, 8, 9 or 10 or more, sometimes up to about 15 or 20 or more amino acids greater in size than a recited polypeptide. Accordingly, a subject peptide may be from about 8 to about 30 amino acids in length. In certain embodiments, a subject peptide is about 9-20 amino acids in length, and usually contains an amino acid sequence described above.

Splenocytes containing B-lymphocytes obtained from the immunized animals, for example mice, are used to generate hybridomas secreting antibodies. In one example, splenocytes containing B-lymphocytes obtained from the immunized animal are fused with mouse myeloma cells, for example, P3×63-Ag-653/Bcl-2 cells, to generate antibody-secreting hybridoma cell cultures. These hybridoma cultures can then be screened for antibodies with specific immunoreactivity against E6 proteins of high-risk HPV strains, such as immunoreactivity to the N-terminus of oncogenic E6 proteins of high-risk HPV strains (Tables 5 and 6). The hybridoma screening may be carried out via various methods and techniques well known in the art including but not limited to enzyme linked immunosorbent assay (ELISA), immunohistochemistry (IHC), and Western blotting. The method of the present invention further comprises cloning hybridoma cells that secret antibodies specific for the N-terminus of oncognenic E6 proteins. The cloning of hybridoma cells may be achieved via different methods, for example, limiting dilution technique, to generate the monoclonal antibodies of the present invention. For example, polyclonal antibodies exhibiting high affinity for E6 proteins are put through a round of limiting dilution to generate monoclonal hybridoma cell lines. The method of the present invention further comprises purifying antibodies that specifically bind to the N-terminus of oncognenic E6 proteins. Many standard techniques may be used to purify antibodies, for example, Protein A, Protein G, or Protein L Agarose protocols may be used for monoclonal antibody purification.

These hybridoma cultures can also be screened for antibodies with specific immunoreactivity against C-terminus of oncogenic E6 proteins of high-risk HPV strains. The hybridoma screening may be carried out via various methods and techniques well known in the art including but not limited to enzyme linked immunosorbent assay (ELISA), immunohistochemistry (IHC), and Western blotting. The method of the present invention further comprises cloning hybridoma cells that secret antibodies specific for the C-terminus of oncognenic E6 proteins. The cloning of hybridoma cells may be achieved via different methods, for example, limiting dilution technique, to generate the monoclonal antibodies of the present invention. The method of the present invention further comprises purifying antibodies that specifically bind to the C-terminus of oncognenic E6 proteins. Many standard techniques may be used to purify antibodies, for example, Protein A, Protein G, or Protein L Agarose protocols may be used for monoclonal antibody purification. The purified antibodies generated via the method of the present invention, which are specific against the C terminus of oncogenic E6 proteins, are further characterized in in vitro antibody-antigen, e.g. antibody-E6 binding assays for assessing their ability to bind and detect oncogenic E6 proteins of high-risk HPV strains. Examples of the purified antibodies of the present invention include mAb 6D9.3 and mAb 1A9.1. Certain hybridoma clones have been deposited at American Type Culture Collection (ATCC). These clones include 4E9.7 (ATCC #PTA-9679), 4E910.2 (ATCC #PTA-9680), 6115.3 (ATCC #PTA-9681), 2119.15 (ATCC #PTA-9871), 7E7.7 (ATCC #PTA-9691), PAEP 8G83 (ATCC #PTA-9685), PAEP 3A10.25 (ATCC #PTA-9686), MMP7-5G11.9 (ATCC #PTA-9682), MMP7-15H8.12 (ATCC #PTA-9683), and PAEP 2G7.1 (ATCC #PTA-9684). These antibodies can be used as capture antibodies or detector antibodies for detecting the presence of E6 proteins in a sample in any immunological based assay formats including but not limited to slide based assay, direct enzyme immunoassay (EIA), RAMAN spectroscopy nanotechnology, immuno-lateral flow assay, ELISA, and bead based cytometric bead array. In some embodiments, these two antibodies are tested as capture antibodies in a sandwich ELISA described herein using biotinylated anti-E6 antibodies as the detector antibodies. The assay design and results are disclosed in details in Example 4 and Table 4.

TABLE 5

| 737BLT | HPV1 Ova | HPV1 Map | 35 pmal | 52 pmal | 56 pmal |
|---|---|---|---|---|---|
| 1B2 | 3.236 | 3.478 | 0.310 | 1.983 | 0.295 |
| 1C8 | 3.018 | 3.199 | 0.199 | 3.104 | 0.211 |
| 1E2 | 2.777 | 3.000 | 2.432 | 0.288 | 0.231 |
| 4B3 | 2.614 | 2.729 | 0.090 | 1.728 | 0.120 |
| 4C3 | 2.848 | 2.825 | 0.108 | 2.554 | 0.154 |
| 4E9 | 3.169 | 2.728 | 0.288 | 2.665 | 0.329 |
| 5D8 | 3.126 | 2.548 | 0.138 | 0.183 | 0.144 |
| 5D9 | 0.775 | 0.434 | 0.018 | 0.026 | 0.018 |
| 5G3 | 3.202 | 2.564 | 0.242 | 0.377 | 0.246 |
| 6F2 | 3.277 | 2.457 | 2.376 | 0.185 | 0.141 |
| 6G8 | 2.875 | 2.673 | 2.749 | 0.271 | 0.273 |
| 7F10 | 3.409 | 3.199 | 0.810 | 2.410 | 0.268 |
| 7G10 | 3.340 | 2.723 | 0.024 | 0.055 | 0.078 |
| 8C1 | OVER | 3.083 | 0.270 | 3.016 | 0.251 |
| 8C2 | OVER | 2.482 | 0.150 | 1.128 | 0.236 |
| 8E8 | OVER | 2.822 | 0.305 | 2.686 | 0.376 |
| 8E9 | OVER | 2.800 | 0.153 | 1.537 | 0.232 |
| 8H6 | 3.003 | 2.207 | 2.633 | 0.463 | 0.482 |
| 9A10 | 3.277 | 2.345 | 2.238 | 0.205 | 0.086 |
| 9C9 | 3.152 | 2.722 | 0.110 | 0.551 | 0.184 |
| 9D6 | OVER | 2.900 | 1.374 | 1.331 | 0.989 |
| 10E9 | 3.352 | 3.021 | 0.178 | 3.324 | 0.154 |
| Media | 0.026 | 0.012 | 0.020 | 0.024 | 0.022 |
| Anti-Ova | 2.806 | NA | NA | NA | NA |
| Anti- MBP | NA | NA | 2.424 | 2.901 | 2.847 |
| Anti-His | NA | NA | NA | NA | NA |

TABLE 6

| 738BLT | HPV2 Ova | HPV2 Map | 35 pmal | 52 pmal | 56 pmal |
|---|---|---|---|---|---|
| 1H1 | 3.283 | OVER | 2.735 | 0.108 | 0.074 |
| 1G11 | 3.418 | OVER | 3.121 | 0.107 | 0.075 |
| 2B1 | 2.537 | 2.38 | 2.276 | 0.061 | 0.066 |
| 4 E9 | 2.807 | 1.911 | 0.054 | 0.167 | 0.060 |
| 4 E10 | 3.146 | OVER | 3.133 | 0.127 | 0.095 |
| 4H5 | OVER | 3.258 | 2.922 | 0.128 | 0.099 |
| 6A7 | 3.191 | 3.321 | 3.140 | 0.183 | 0.072 |
| 6C3 | 3.142 | 3.388 | 3.240 | 0.167 | 0.098 |
| 6 E6 | 3.117 | 3.486 | 3.353 | 0.179 | 0.122 |
| 6F11 | OVER | 3.382 | 3.302 | 0.152 | 0.186 |
| 6H5 | 3.171 | 2.853 | 2.467 | 0.389 | 0.316 |
| 7 E7 | 3.21 | 3.472 | 2.963 | 0.125 | 0.105 |
| 7H12 | OVER | 3.288 | 2.916 | 0.106 | 0.100 |
| 8 E8 | 1.852 | 0.993 | 1.334 | 1.257 | 0.727 |
| 8F9 | 3.176 | 3.34 | 3.069 | 0.203 | 0.161 |
| 8G10 | 3.075 | 3.397 | 2.910 | 0.139 | 0.075 |
| 8H5 | 1.343 | 0.034 | 0.139 | 0.202 | 0.110 |
| 8H6 | OVER | 0.034 | 2.990 | 0.241 | 0.156 |
| 8H9 | OVER | 0.034 | 3.111 | 0.141 | 0.094 |
| 9D7 | 1.965 | 0.034 | 0.356 | 0.066 | 0.092 |
| 9G6 | 3.33 | 0.034 | 3.210 | 0.142 | 0.087 |
| 9H10 | 3.175 | 0.034 | 2.952 | 0.243 | 0.194 |
| 10G10 | 3.275 | 0.034 | 2.952 | 0.243 | 0.194 |
| Media | 0.026 | 0.034 | 0.020 | 0.024 | 0.022 |
| Anti-Ova | 2.806 | 0.034 | NA | NA | NA |
| Anti- MBP | NA | 0.034 | 2.424 | 2.901 | 2.847 |
| Anti-His | NA | 0.034 | NA | NA | NA |

The purified antibodies generated via the method of the present invention, which are specific against the N-terminus of oncogenic E6 proteins, are further characterized in in vitro antibody-antigen, e.g. antibody-E6 binding assays for assessing their ability to bind and detect oncogenic E6 proteins of high-risk HPV strains. In some embodiments, each monoclonal antibody of the present invention is tested for cross reactivity in Western blots against purified E6 proteins from multiple HPV strains including HPV16, 18, 31, 33, 35, 45, 52, 56, 58, 69, 11, and 6b (Table 7).

TABLE 7

| | 16 | 18 | 33 | 31 | 35 | 45 | 52 | 56 | 58 | 69 | 11 | 6b |
|---|---|---|---|---|---|---|---|---|---|---|---|---|
| 737 BLT | | | | | | | | | | | | |
| 1B2.2 | 2 | 2 | 3 | 4 | 3 | 0 | 3 | 1 | 3 | 0 | 0 | 0 |
| 1B2.27 | 2 | 3 | 3 | 4 | 3 | 0 | 3 | 1 | 3 | 0 | 0 | 0 |
| 7F10.3 | 2 | 4 | 4 | 4 | 4 | 0 | 4 | 1 | 4 | 0 | 0 | 0 |
| 7F10.29 | <1 | 2 | 4 | 4 | 4 | <1 | 4 | 2 | 4 | 0 | 0 | 0 |
| 738 BLT | | | | | | | | | | | | |
| 4E9.7 | <1 | 3 | 0 | 0 | 0 | 3 | 0 | 0 | 0 | 0 | 0 | 0 |
| 4E9.10 | <1 | 3 | 0 | 0 | 0 | 3 | 0 | 0 | 0 | 0 | 0 | 0 |
| 6H5.3 | 0 | 0 | 0 | 0 | 4 | 0 | 0 | 0 | 0 | 0 | 0 | 0 |
| 6H5.8 | 0 | 0 | 0 | 0 | 4 | 0 | 0 | 0 | 0 | 0 | 0 | 0 |
| 4E10.2 | 0 | 4 | 0 | 0 | 4 | 4 | 0 | 0 | 0 | 0 | 0 | 0 |
| 4E10.13 | 0 | 4 | 0 | 0 | 4 | 4 | 0 | 0 | 0 | 0 | 0 | 0 |
| MEDIA | 0 | 0 | 0 | 0 | 1 | 0 | 0 | 0 | 0 | 0 | 0 | 0 |
| AVC | | | | | | | | | | | | |
| 3D5 AV | NT | 4 | NT | NT | NT | 4 | NT | NT | NT | NT | NT | NA |
| 4C6 AV | NT | 4 | NT | NT | 3 | NT | NT | NT | NT | NT | NT | NA |
| 4H1 AV | NT | NT | 4 | NT | NT | NT | 3 | NT | 4 | NT | 3 | NA |
| 7F10 AV | NT | NT | NT | 4 | NT | NT | NT | NT | NT | NT | NT | NA |
| 6F4 AV | 3 | 4 | NT | NT | NT | NT | NT | NT | NT | 2 | NT | NA |

These antibodies are further characterized in an in vitro antibody pair sandwich assay to determine complementary epitopes on E6 oncoproteins of high-risk HPV strains (Table 8). Examples of the purified antibodies of the present invention include but are not limited to 4E9.7, 4E10.2, 6H5.3, 2H9.15, 7F10.3, 4E9.7, 1B2.27, and those listed in Tables 5 and 6. These antibodies can be used as capture antibodies or detector antibodies for detecting the presence of E6 proteins in a sample in any immunological based assay formats including but not limited to slide based assay, direct enzyme immunoassay (EIA), Ramon spectroscopy nanotechnology, immuno-lateral flow assay, ELISA, and bead based cytometric bead array. In some embodiments, the subject antibodies are tested as capture antibodies in a sandwich ELISA described herein using another E6-binding partner, for example, another anti-E6 antibody or a PDZ domain containing polypeptide, as the detector. In other embodiments, the subject antibodies are tested as detector antibodies in a sandwich ELISA described herein using another E6-binding partner, for example, another anti-E6 antibody or a PDZ domain containing polypeptide, as the capture agent for E6 protein. In these examples, the other anti-E6 antibody may bind to the C-terminus of E6 protein or a region on E6 other than the N-terminal binding site to which the subject antibody binds. The PDZ domain polypeptides, such as MAGI-1 and MUPP1, typically bind to the PDZ domain motif in the C-terminal region of oncogenic E6 proteins.

TABLE 8

| | | |
|---|---|---|
| Capture | Detection | Ratio |

HPV 16 Bac pairs

| | | | |
|---|---|---|---|
| 738 - 4E9.7 | AV - 4C6 | 2.4 | |
| 744 - 8F1 | AV - 4C6 | 3.6 | |
| 876C2 - 11E10.30 | AV - 4C6 | 3.5 | |
| 876IS - 3C12.23 | AV - 4C6 | 2.8 | |
| 738 - 4E9.7 | AV - 8G11 | 3.5 | |
| 743 - 5H12.13 | AV - 8G11 | 2.2 | |
| 743 - 10B4.15 | AV - 8G11 | 2.7 | |
| 744 - 8F1 | AV - 8G11 | 5.3 | |
| 876C2 - 11E10.30 | AV - 8G11 | 5.1 | |
| 876IS - 3C12.23 | AV - 8G11 | 3.7 | |
| 876IS - 5G4 | AV - 8G11 | 2.2 | |
| 876IS - 7F3 | AV - 8G11 | 2.3 | |
| 738 - 4E9.7 | 743 - 6D3.5 | 2.4 | |
| 744 - 8F1 | 743 - 6D3.5 | 3.4 | |
| 876C2 - 11E10.30 | 743 - 6D3.5 | 3.1 | |
| 876IS - 3C12.23 | 743 - 6D3.5 | 2.6 | |
| 738 - 4E9.7 | 743 - 6D8.30 | 3.0 | |
| 743 - 5H12.13 | 743 - 6D8.30 | 2.1 | |
| 743 - 10B4.15 | 743 - 6D8.30 | 2.6 | |
| 744 - 8F1 | 743 - 6D8.30 | 4.0 | * |
| 876C2 - 11E10.30 | 743 - 6D8.30 | 4.0 | * |
| 876IS - 3C12.23 | 743 - 6D8.30 | 3.5 | * |
| 876IS - 5G4 | 743 - 6D8.30 | 2.2 | |
| 876IS - 7F3 | 743 - 6D8.30 | 2.6 | |
| AV - 4C6 | 743 - 8H11.23 | 2.1 | |
| 738 - 4E9.7 | 743 - 8H11.23 | 2.9 | |
| 743 - 5H12.13 | 743 - 8H11.23 | 2.2 | |
| 743 - 10B4.15 | 743 - 8H11.23 | 2.2 | |
| 744 - 8F1 | 743 - 8H11.23 | 4.8 | * |
| 876C2 - 11E10.30 | 743 - 8H11.23 | 4.1 | * |
| 876IS - 3C12.23 | 743 - 8H11.23 | 3.2 | |
| 876IS - 5G4 | 743 - 8H11.23 | 2.3 | |
| 876IS - 7F3 | 743 - 8H11.23 | 2.5 | |
| 738 - 4E9.7 | 876IS - 5G4 | 2.9 | |
| 743 - 5H12.13 | 876IS - 5G4 | 2.2 | |
| 743 - 6D8.30 | 876IS - 5G4 | 2.2 | |
| 743 - 10B4.15 | 876IS - 5G4 | 2.6 | |
| 744 - 8F1 | 876IS - 5G4 | 3.4 | |
| 876C2 - 11E10.30 | 876IS - 5G4 | 3.9 | * |
| 876IS - 3C12.23 | 876IS - 5G4 | 3.5 | * |
| 876IS - 5G4 | 876IS - 5G4 | 2.2 | |
| 876IS - 7F3 | 876IS - 5G4 | 2.4 | |

HPV 16 HEK pairs

| | | |
|---|---|---|
| 876IS - 1D7 | 876IS - 7F3 | 2.0 |

HPV 18 Bac pairs

| | | |
|---|---|---|
| AV - 3D5 | 744 - 1D3 | 2.5 |
| AV - 5F9 | 744 - 1D3 | 3.9 |
| AV - 15F4 | 744 - 1D3 | 4.1 |
| 876C2 - 11E10.30 | 744 - 1D3 | 2.1 |
| AV - 3D5 | 744 - 3G9 | 2.5 |
| AV - 5F9 | 744 - 3G9 | 3.9 |
| AV - 15F4 | 744 - 3G9 | 4.3 |
| 876C2 - 11E10.30 | 744 - 3G9 | 2.2 |
| AV - 5F9 | 744 - 7D11 | 2.8 |
| AV - 15F4 | 744 - 7D11 | 3.5 |
| AV - 5F9 | 744 - 10E11.10 | 2.2 |
| AV - 15F4 | 744 - 10E11.10 | 2.4 |

* MBR pairs
******* purified MBR pairs

In general, methods for making antibodies, particular monoclonal antibodies, are well known in the art and described in various well known laboratory manuals (e.g., Harlow et al., Antibodies: A Laboratory Manual, First Edition (1988) Cold spring Harbor, N.Y.; Harlow and Lane, Using Antibodies: A Laboratory Manual, CSHL Press (1999) and Ausubel, et al., Short Protocols in Molecular Biology, 3rd ed., Wiley & Sons, (1995)). Accordingly, given the peptide sequences, methods for making the subject antibodies do not need to be described herein in any great detail. Any fragment of a longer full-length E6 protein that contains a subject common motif (e.g., the full length protein), a full length E6 protein, or a fusion protein thereof may be used to make the subject antibodies. In certain embodiments, a full length E6 protein, a peptide containing a recited sequence, or a chemically modified (e.g., conjugated) derivative or fusion thereof (e.g., a MBP or GST fusion), may be used as an antigen. In certain embodiments, a nucleic acid encoding the polypeptide may be employed, or a mixture of different polypeptides (e.g., a mixture of E6 polypeptides, each polypeptide from a different HPV strain) may be used as an antigen (Michel (2002) Vaccine 20:A83-A88). Accordingly an antigen is mixed with an adjuvant, and a suitable non-human animal (e.g., a mouse, chicken, goat, rabbit, hamster, horse, rat or guinea pig, etc.) is immunized using standard immunization techniques (e.g., intramuscular injection) and once a specific immune response of the has been established, blood from the animal may be collected and polyclonal antisera that specifically binds to described peptides may be isolated. In many cases, cells from the spleen of the immunized animal are fused with a myeloma cell line, and, after fusion, the cells are grown in selective medium containing e.g., hypoxanthine, aminopterin, and thymidine (HAT), to select for hybridoma growth, and after 2-3 weeks, hybridoma colonies appear. Supernatants from these cultured hybridoma cells are screened for antibody secretion, usually by enzyme-linked immunosorbent assay (ELISA) or the like, and positive clones secreting monoclonal antibodies specific for the antigen can be selected and expanded according to standard procedures.

Accordingly, depending on the antibodies desired, a suitable animal is immunized with a subject peptide or a mixture of subject peptides (e.g., a mixture of 2, 3, 4, 5 about 6 or more, about 10 or more or about 15 or more, usually up to about 20 or 30 or more peptides described above). Antibodies are usually isolated from the animal and tested for binding to different HPV E6 proteins using standard methods (e.g., ELISA, western blot, etc.). In many embodiments, therefore, antibodies will be screened for binding to E6 proteins from HPV strains 16 and 18, HPV strains 16, 18, 31, 33 and 45, or, in certain embodiments, HPV strains 16, 18, 26, 30, 31, 34, 39, 45, 51, 52, 53, 58, 59, 66, 68, 69, 70, 73, and 82, and maybe others. Accordingly, antibodies that bind to, i.e., cross-react with, E6 proteins from more than one strain of HPV may be identified, and permanent cell lines producing those antibodies may be established using known methods. In other words, antibodies are usually tested for binding to more than one antigen, and those antigens are usually E6 proteins from various HPV strains, or fragments thereof. In most embodiments, the antibodies will be tested for binding to antigens in native and denatured states. Antibodies that bind to a plurality of E6 proteins have desirable binding properties, and, accordingly, find use in the subject methods.

In one example, polynucleotides encoding the N-terminal region of E6 proteins of high-risk HPV types listed hereinabove may be chemically synthesized or cloned via RT-PCR from cervical cancer cell lines. Both KLH-E6 and ovalbumin-E6 (OVA-E6) fusion protein types can be used. Production of KLH-E6 and OVA-E6 proteins can be by standard protocols in the art. Proteins can be expressed in DH5α E. coli using IPTG driven induction. A 2-hour induction at 37° C. may yield KLH-E6 or OVA-E6 peptides at about 1 mg/L, whereas induction overnight at 20° C. and purification including rebinding of protein to the gel matrix may result in a final yield of 2-10 mg/L. In addition, protein expression at a lower temperature than 37° C. may allow for better protein folding such that the native epitopes are better preserved, increasing the likelihood of generating antibodies that are specific against E6 N-terminus of multiple oncogenic HPV strains, i.e. pan-specific antibodies against oncogenic E6 proteins. Purity of KLH-E6 or OVA-E6 proteins is estimated to be >90% based on PAGE analysis. E6 fusion proteins can be used as the immunogens.

Mice can then be immunized with each of the oncogenic HPV E6 proteins, such as the N-terminal sequences. A variety of immunization protocols including varying antigen doses (100 μg-10 μg), adjuvants (CFA/IFA, poly(I)-poly(C), CpG+Alum) and routes (subcutaneous, intraperitoneal) are tested. A service facility for animal care, handling of immunizations and sera collection was contracted (Josman, Napa, Calif.). Immunization projects are set up with 5-15 mice each. Sera of immunized mice are tested in ELISA against the recombinant E6 protein. Mice showing sufficiently high titers (OD above 1 at 1:1000 dilution) against E6 in their sera are selected for fusions. To increase the frequency of hybridomas secreting anti-E6 antibodies, the E6 protein used in the final boost contained a different tag from that used during the immunization, for example, glutathione-S-transferase (GST)-E6 immunizing peptide is used in the boost when immunizations occurs with maltose binding protein (MBP)-E6 immunizing peptide, and vice versa. Detailed immunization methods for generating anti-E6 antibodies are disclosed in U.S. Pat. No. 7,399,467, which is herein incorporated by reference in its entirety.

In one example, polynucleotides encoding the C-terminal region of E6 proteins of high-risk HPV types listed hereinabove may be chemically synthesized or cloned via RT-PCR from cervical cancer cell lines. Both maltose-binding-protein-E6 (MBP-E6) and glutathione-S-transferase-E6 (GST-E6) fusion protein types can be used. Production of GST-E6 and MBP-E6 proteins can be by standard protocols recommended by the suppliers (Amersham and New England Biolabs, respectively). Proteins are expressed in DH5α E. coli using IPTG driven induction. A 2-hour induction at 37° C. yields GST-E6 or MBP-E6 recombinant proteins at about 1 mg/L, whereas induction overnight at 20° C. and purification including rebinding of protein to the gel matrix may result in a final yield of 2-10 mg/L. In addition, protein expression at a lower temperature than 37° C. may allow for better protein folding such that the native epitopes are better preserved, increasing the likelihood of generating antibodies that are specific against E6 C-terminus of multiple oncogenic HPV strains, i.e. pan-specific antibodies against oncogenic E6 proteins. Purity of MBP-E6 proteins is estimated to be >90% based on PAGE analysis. Recombinant E6 fusion proteins can be used as the immunogens.

Mice can then be immunized with each of the oncogenic HPV E6 protein C terminal sequences. A variety of immunization protocols including varying antigen doses (100 μg-10 μg), adjuvants (CFA/IFA, poly(I)-poly(C), CpG+Alum) and routes (subcutaneous, intraperitoneal) are tested. A service facility for animal care, handling of immunizations and sera collection was contracted (Josman, Napa, Calif.). Immunization projects are set up with 2-15 mice each. Sera of immunized mice are tested in ELISA against the recombinant E6 protein. Mice showing sufficiently high titers (OD above 1 at 1:1000 dilution) against E6 in their sera are selected for fusions. To increase the frequency of hybridomas secreting anti-E6 antibodies, the recombinant E6 protein used in the final boost contained a different tag from that used during the immunization, for example, GST-E6 immunizing peptide is used in the boost when immunizations occurs with MBP-E6 immunizing peptide, and vice versa. In some embodiments, the immunizing peptide containing a T cell epitope does not contain a tag.

Exemplary peptides of E6 suitable for immunizations are described in Table 1. The peptides are shown as a “consensus” sequence (i.e. peptides in which one of several amino acids may exist at one or more positions) in order to indicate that any one or a mixture of different peptides that are described by the consensus could be used to make the subject antibodies. Accordingly, when a consensus sequence is described, every individual peptide that falls within the consensus should be considered explicitly described. In particular embodiments, exemplary species of peptide encompassed by the consensus sequences have a sequence found in a naturally-occurring HPV E6 protein. Such exemplary sequences can be identified as sequences starting at the amino acid positions defined by the third column of Table 1, “Starting AA” of particular HPV types “HPV type”, and corresponding positions of other HPV E6 proteins (i.e., those positions that are aligned with the positions indicated in Table 1).

In one example, polynucleotides encoding E6 proteins of high-risk HPV types listed hereinabove may be chemically synthesized (DNA 2.0, Menlo Park, Calif.) or cloned via RT-PCR from cervical cancer cell lines. Both maltose-binding-protein-E6 (MBP-E6) and glutathione-S-transferase-E6 (GST-E6) fusion protein types can be used. Production of GST-E6 and MBP-E6 proteins can be by standard protocols recommended by the suppliers (Amersham and New England Biolabs, respectively). Proteins are expressed in DH5α E. coli using IPTG driven induction. A 2-hour induction at 37° C. yields GST-E6 or MBP-E6 recombinant proteins at about 1 mg/L, whereas induction overnight at 20° C. and purification including rebinding of protein to the gel matrix may result in a final yield of 2-10 mg/L. Purity of MBP-E6 proteins is estimated to be >90% based on PAGE analysis. Recombinant E6 fusion proteins can be used as the immunogens.

As is well known in the art, the subject antibodies may be conjugated to a detectable label, or may be part of a signal generating system, as described above. A detailed disclosure of generating antibodies against E6 protein of HPV including immunization of animals, fusion, screening and cloning of hybridomas secreting monoclonal antibodies against E6 protein can be seen in U.S. Pat. No. 7,399,467, which is herein incorporated by reference in its entirety.

Accordingly, using the methods set forth above, an antibody composition for detecting a plurality of HPV E6 proteins is provided. In certain embodiments, a mixture of different antibodies that recognize at least 5, 7, 9, 12, 15, 20 or 24 different strains of HPV may be employed. The composition may contain a combination of antibodies that recognize at least 3 different oncogenic E6 proteins. The composition may contain 1, 2, 3, 4, or 5 or more different antibodies, each antibody of the composition recognizing at least one (e.g., 2, 3, about 5, about 10, etc.) E6 proteins. Collectively, the antibodies bind to all or a portion of the E6 proteins of multiple HPV strains disclosed herein. The antibodies may be mixed, or separate from each other, i.e., in different vessels.

Any of the above-described antibodies may bind to an epitope set forth in Table 9.

TABLE 9
Epitopes

| | PV | tarting |
|---|---|---|
| (1) Sequence | type | AA |
| (4) (K/R)-(K/R)-R-F-H-(N/K/S/E/R)-I-(A/S) | 9 | 24 |
| (7) F-H-(N/K/S/E/R)-I-(A/S)-(G/H)-X-(W/Y) | 9 | 27 |
| (10) H-(N/K/S/E/R)-I-(A/S)-(G/H)-(R/Q)-(W/Y)-(T/K/R) | 9 | 28 |
| (13) P-(E/A/Q)-E-K-(Q/L/K/R)-(R/K/L)-(HN/I/L)-(V/L/C) | 6 | 12 |
| (16) (G/H)-(R/Q/T/M/G/A/Y/H/S/N/I)-(W/Y/F)-(T/R/K/A)-G-(R/Q/S/L)-C- | 9 | 32 |
| (R/L/M/A/T) | | |
| (19) (W/Y/F)-(T/R/K/A)-G-(R/Q/S/L)-C-(R/L/M/A/T)-(L/R/A/T)- | 9 | 34 |
| (N/R/S/A/Q/G) | | |
| (22) G-(R/Q/S/L)-C-(R/L/M/A/T)-(L/R/A/T)-(N/R/S/A/Q/G)-C-(W/C/R) | 9 | 36 |
| (25) (R/K)-P-(R/Y)-(KJT/S)-(LN)-(H/P)-(D/E/H/Q)-L | 9 | 0 |
| (28) (M/R/L)-F-(E/Q/D/H)-(D/N)-(P/T)-(Q/R/A/E/T)-(E/Q)-(R/K) | 9 | |
| (31) (D/N)-(P/T)-(Q/R/A/E/T)-(E/Q)-(R/K)-(R/K)-P-(RJY) | 9 | |
| (34) (LN)-(H/P)-(D/E/Q)-L-(C/S)-(E/T/Q)-(EN/A/T)-(LN)-(N/E/D) | 9 | 4 |
| (37) (D/E/N)-(LN/I)-(Q/E/R/T)-(LN/I)-(Q/N/D/S/A/N)-C-V-(F/Y/E)- | 9 | 6 |
| (40) L-(L/S)-I-R-C-(I/Y/H/L/M)-(R/I/C)-C | 9 | 01 |
| (43) (R/I/C)-C-(Q/L)-(K/R)-P-L-(C/T/G/N)-P | 9 | 07 |
| (46) (IQR)-P-L-(C/T/G/N)-P-(E/A/Q)-E-K | 9 | 10 |
| (49) P-(E/A/Q)-E-K-(Q/L/K)-(R/L/K)-(H/I)-(LN/C) | 6 | 12 |
| (52) K-(Q/L/K)-(R/L/K)-(H/I)-(L/V/C)-(D/E/N)-(E/D/Y/L/K/S)-(K/N) | 6 | 15 |
| (55) (LN/C)-(D/E/N)-(E/D/Y/L/K/S)-(K/N)-(K/R)-R-F-H | 6 | 19 |
| (58) I-(A/S)-(G/H)-(R/Q)-(W/Y)-(T/KJR)-G-(R/Q/L/S) | 6 | 28 |
| (61) (W/Y)-(T/K/R)-G-(R/Q/L/S)-C-(M/A/L/R/T)-(N/S/A/R)-C | 6 | 32 |

The antibodies of the invention may be screened for immunospecific binding to oncogenic E6 proteins by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and cellular immunostaining (fixed or native) assays to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety).

Certain hybridomas that produce the monoclonal antibodies described above and below may be deposited at the ATCC, for example, hybridoma cell lines 4E9.7 (PTA-9679), 4E10.2 (PTA-9680, and 6H5.3 (PTA-9681). Any of the deposited hybridomas, the antibodies produced by those hybridomas, as well as other antibodies that bind the same epitopes as the antibodies produced by those hybridomas, are also embodiments of this invention and may be claimed herein. Such antibodies may be employed in any of the methods described herein.

To further characterize or confirm the specificity of the subject antibodies, the antibodies of the invention may be screened using immunocytochemisty methods on cells (e.g., mammalian cells, such as CHO cells) transfected with a vector enabling the expression of an antigen or with vector alone using techniques commonly known in the art. Antibodies that bind antigen transfected cells, but not vector-only transfected cells, are antigen specific. In certain embodiments, the assay is an antigen capture assay, and an array or microarray of antibodies may be employed for this purpose. Methods for making and using microarrays of polypeptides are known in the art (see e.g. U.S. Pat. Nos. 6,372,483, 6,352,842, 6,346,416 and 6,242,266).

Examples of peptides that may be used for screening the specific antibodies of the present invention include but are not limited to those peptides listed in Table 2.

Any of the deposited hybridomas, the antibodies produced by those hybridomas, as well as other antibodies that bind to the same epitopes as the antibodies produced by those hybridomas, are also embodiments of this invention and may be claimed herein. Such antibodies may be employed in any of the methods described herein.

Detecting HPV E6 Proteins in a Sample

In one aspect, the present invention provides a method of detecting an E6 protein of a HPV strain in a sample, comprising the steps of contacting an antibody which specifically binds to amino-terminus (N-terminus) of oncognenic E6 proteins of at least two HPV strains with the sample, and detecting any binding of the antibody to the E6 protein in the sample; wherein binding of the antibody to the E6 protein in the sample indicates the presence of at least one HPV strain in the sample. In some embodiments, the HPV strain is an oncogenic strain. Since the subject antibodies target the N-terminus of E6 protein, the subject antibodies can be used in combination with antibodies or PDZ domain containing polypeptides that bind to the PDZ domain in the C-terminal region of E6 proteins. The antibodies of the present invention may avoid masking of the PDZ binding domain on E6 proteins.

In one aspect, the present invention provides a method of detecting an E6 protein of an oncogenic HPV strain in a sample, comprising the steps of contacting an antibody which specifically binds to carboxyl-terminus (C-terminus) of oncognenic E6 proteins of at least two HPV strains with the sample, and detecting any binding of the antibody to the E6 protein in the sample; wherein binding of the antibody to the E6 protein in the sample indicates the presence of at least one oncogenic HPV strain in the sample.

In general, the subject method involves contacting a sample suspected of having HPV with a capture antibody that specifically binds to an oncogenic E6 protein at its N or C terminus, contacting the sample with a detection antibody specific against the same E6 protein, and assessing any binding of the antibody mixture to the sample. The antibody of the present invention may be used as either a capture or a detector bioreagent for detection of E6 proteins. In most embodiments, binding of the subject antibody to the E6 protein indicates the presence of at least one high-risk HPV strain in the sample.

The antibodies of the invention may be used for immunospecific binding to the N-terminus or C-terminus of oncogenic E6 proteins of at least two high-risk HPV strains by any method known in the art. The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, immunohistochemistry, and cellular immunostaining (fixed or native) assays to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

In some embodiments, the method of the present invention utilizes an enzyme linked immunosorbent assay (ELISA), preferably a sandwich ELISA, to detect the presence of oncogenic E6 proteins bound to the antibody composition of the present invention. As used herein, the terms “sandwich”, “sandwich ELISA”, “sandwich diagnostic” and “capture ELISA” all refer to the concept of detecting a biological polypeptide with two different test agents. In some embodiments, the sandwich assay comprises two antibodies that bind specifically to an oncogenic E6 protein, for example, the E6 protein of at least two high-risk HPV strains. In some embodiments, the subject antibody is used as an immobilized antibody to capture E6 protein in an enzyme-linked immunosorbent assay (ELISA). For example, such ELISA may comprise (a) contacting the subject antibody with the sample; (b) contacting the E6 protein that is bound to the subject antibody with a second antibody or a PDZ domain containing polypeptide that specifically binds to the E6 protein at a binding site that is different from that of the subject antibody; and (c) detecting binding of the second antibody or the PDZ domain polypeptide to the E6 protein, thereby detecting the presence of the E6 protein in the sample. In other embodiments, the antibody is used as a detector antibody to detect E6 protein that is bound to a capture antibody specific for the E6 protein or a PDZ domain containing polypeptide in an enzyme-linked immunosorbent assay (ELISA). For example, such ELISA may comprise (a) contacting the sample with an antibody or a PDZ domain containing polypeptide that specifically binds to E6 protein at a binding site that is different from that of the subject antibody; (b) contacting the E6 protein that is bound to the antibody or the PDZ domain polypeptide with the subject antibody; and (c) detecting binding of the subject antibody to the E6 protein, thereby detecting the presence of the E6 protein in the sample. The capture antibody or the PDZ domain polypeptide may or may not be immobilized to a substrate.

ELISA is a well known technique in the art. Briefly, ELISA involves preparing antigen, coating the well of a 96 well multiwell plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISA, the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art.

As discussed supra, it will be appreciated that many of the steps in the above-described assays can be varied, for example, various substrates can be used for binding the antibodies; antibodies recognizing different E6 epitopes can be used; different labels for detecting antibody-E6 interactions can be employed; and different ways of detection can be used.

The antibody detection assays can employ a variety of surfaces to bind the antibodies or antibody fragments. For example, a surface can be an “assay plate” which is formed from a material (e.g. polystyrene) which optimizes adherence of protein thereto. Generally, the individual wells of the assay plate will have a high surface area to volume ratio and therefore a suitable shape is a flat bottom well (where the proteins of the assays are adherent). Other surfaces include, but are not limited to, polystyrene or glass beads, polystyrene or glass slides, cellulose, nitrocellulose, papers, dipsticks, plastics, films and the like.

For example, the assay plate can be a “microtiter” plate. The term “microtiter” plate when used herein refers to a multiwell assay plate, e.g., having between about 30 to 200 individual wells, usually 96 wells. Alternatively, high-density arrays can be used. Often, the individual wells of the microtiter plate will hold a maximum volume of about 250 ul. Conveniently, the assay plate is a 96 well polystyrene plate (such as that sold by Becton Dickinson Labware, Lincoln Park, N.J.), which allows for automation and high throughput screening. Other surfaces include polystyrene microtiter ELISA plates such as that sold by Nunc Maxisorp, Inter Med, Denmark. Often, about 50 ul to 300 ul, more preferably 100 ul to 200 ul, of an aqueous sample comprising buffers suspended therein will be added to each well of the assay plate.

The detectable labels of the invention can be any detectable compound or composition which is conjugated directly or indirectly with a molecule, for example, the second anti-E6 antibody of the present invention. The label can be detectable by itself (e.g., radioisotope labels or fluorescent labels) or, in the case of an enzymatic label, can catalyze a chemical alteration of a substrate compound or composition which is detectable. The preferred label is an enzymatic one which catalyzes a color change of a non-radioactive color reagent.

Direct labels include but are not limited to radioisotopes (e.g., ¹²⁵I; ³⁵S, and the like); enzymes whose products are detectable (e.g., luciferase, 11-galactosidase, horseradish peroxidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin; fluorescent proteins; and the like. Fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; and the like.

Sometimes, the label is indirectly conjugated with the antibody. One of skill is aware of various techniques for direct and indirect conjugation. For example, the antibody can be conjugated with biotin and any of the categories of labels mentioned above can be conjugated with avidin, or vice versa (see also “A” and “G” assay above). Biotin binds selectively to avidin and thus, the label can be conjugated with the antibody in this indirect manner. See, Ausubel, supra, for a review of techniques involving biotin-avidin conjugation and similar assays. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

Assay variations can include different washing steps. By “washing” is meant exposing the solid phase to an aqueous solution (usually a buffer or cell culture media) in such a way that unbound material (e.g., non-adhering cells, non-adhering capture agent, unbound ligand, receptor, receptor construct, cell lysate, or HRP antibody) is removed therefrom. To reduce background noise, it is convenient to include a detergent (e.g., Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) can be required.

Various buffers can also be used in the sandwich detection assays of the present invention. For example, various blocking buffers can be used to reduce assay background. The term “blocking buffer” refers to an aqueous, pH buffered solution containing at least one blocking compound which is able to bind to exposed surfaces of the substrate which are not coated with a PL or PDZ-containing protein. The blocking compound is normally a protein such as bovine serum albumin (BSA), gelatin, casein or milk powder and does not cross-react with any of the reagents in the assay. The block buffer is generally provided at a pH between about 7 to 7.5 and suitable buffering agents include phosphate and TRIS.

Various enzyme-substrate combinations can also be utilized in detecting the first antibody-E6-second antibody sandwich interactions. Examples of enzyme-substrate combinations include but are not limited to, for example:

(i) Horseradish peroxidase (HRP or HRPO) with hydrogen peroxide as a substrate, wherein the hydrogen peroxide oxidizes a dye precursor (e.g. orthophenylene diamine [OPD] or 3,3′,5,5′-tetramethyl benzidine hydrochloride [TMB]) (as described above).

(ii) alkaline phosphatase (AP) with para-Nitrophenyl phosphate as chromogenic substrate.

(iii) Beta-D-galactosidase (Beta D-Gal) with a chromogenic substrate (e.g. p-nitrophenyl-Beta-D-galactosidase) or fluorogenic substrate 4-methylumbelliferyl-Beta-D-galactosidase.

Numerous other enzyme-substrate combinations are available to those skilled in the art. For a general review of these, see U.S. Pat. Nos. 4,275,149 and 4,318,980, both of which are herein incorporated by reference in their entirety.

Preferred embodiments of the antibody ELISA are described in detail herein. However, it will be appreciated by ordinarily skilled practitioners that these assays can be modified in numerous ways while remaining useful for the purposes of the present invention. In some embodiments, the capture antibody is immobilized on a solid surface. The substrate to which the antibody is bound may in any of a variety of forms, e.g., a microtiter dish, a test tube, a dipstick, a microcentrifuge tube, a bead, a spinnable disk, a permeable or semi-permeable membrane, and the like. Suitable materials include glass, plastic (e.g., polyethylene, PVC, polypropylene, polystyrene, and the like), protein, paper, carbohydrate, lipid monolayer or supported lipid bilayer, films and other solid supports. Other materials that may be employed include ceramics, metals, metalloids, semiconductive materials, cements and the like.

In some embodiments, the capture antibodies are organized as an array. The term “array,” as used herein, refers to an ordered arrangement of immobilized proteins, in which particular different proteins (i.e., recognizing different E6 proteins of different HPV strains, or different epitopes of E6 protein) are located at different predetermined sites on the substrate. Because the location of particular antibodies on the array is known, binding at that location can be correlated with binding to the antigen situated at that location. Immobilization of antibodies on beads (individually or in groups) is another particularly useful approach. In one embodiment, individual antibodies are immobilized on beads. In one embodiment, mixtures of distinguishable beads are used. Distinguishable beads are beads that can be separated from each other on the basis of a property such as size, magnetic property, color (e.g., using flow cytometry) or affinity tag (e.g., a bead coated with protein A can be separated from a bead not coated with protein A by using IgG affinity methods). Binding to particular HPV protein may be determined.

When antibody-mediated immobilization methods are used, glass and plastic are especially useful substrates. The substrates may be printed with a hydrophobic (e.g., Teflon) mask to form wells. Preprinted glass slides with 3, 10 and 21 wells per 14.5 cm² slide “working area” are available from, e.g., SPI Supplies, West Chester, Pa.; also see U.S. Pat. No. 4,011,350). In certain applications, a large format (12.4 cm×8.3 cm) glass slide is printed in a 96 well format is used; this format facilitates the use of automated liquid handling equipment and utilization of 96 well format plate readers of various types (fluorescent, colorimetric, scintillation). However, higher densities may be used (e.g., more than 10 or 100 polypeptides per cm²). See, e.g., MacBeath et al, 2000, Science 289:1760-63. Typically, antibodies are bound to substrates (e.g., glass substrates) by adsorption. Suitable adsorption conditions are well known in the art and include incubation of 0.5-50 ug/ml (e.g., 10 ug/ml) mAb in buffer (e.g., PBS, or 50 to 300 mM Tris, MOPS, HEPES, PIPES, acetate buffers, pHs 6.5 to 8, at 4° C.) to 37° C. and from 1 hr to more than 24 hours. Proteins may be covalently bound or noncovalently attached through nonspecific bonding. If covalent bonding between the protein and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

The binding and detection of E6 protein by the subject antibody may be via any immunological based assay, such as immunoprecipitation, western blotting, enzyme immunoassays (EIA), RAMAN spectroscopy, lateral flow, and cytometric bead array (CBA). These assays are well known in the art and are briefly described herein, infra.

Immunoprecipitation protocols generally involve lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads).

Western blot analysis generally involves preparation of protein samples followed by electrophoresis of the protein samples in a polyacrylamide gel (e.g., 8%-20% SDS-PAGE depending on the molecular weight of the antigen), and transfer of the separated protein samples from the polyacrylamide gel to a membrane such as nitrocellulose, PVDF or nylon. Following transfer, the membrane is blocked in blocking solution (e.g., PBS with 3% BSA or non-fat milk), washed in washing buffer (e.g., PBS-Tween 20), and incubated with primary antibody (the antibody of interest) diluted in blocking buffer. After this incubation, the membrane is washed in washing buffer, incubated with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I), and after a further wash, the presence of the antigen may be detected. One of skill in the art would be knowledgeable as to the parameters that can be modified to increase the signal detected and to reduce the background noise.

Surface-enhanced Raman scattering nanotechnology has been a valuable tool for surface and interfacial research. The enhancement associated with this process overcomes the low traditional low sensitivity problem in the normal Raman scattering. One of the specific applications is that it makes detection of minute quantities of biochemical chemicals, e.g., DNA, RNA, proteins, on metal surfaces feasible for bio-detection and bio-fingerprinting. In some embodiments, Ramon spectroscopy nanotechnology may be used to detect binding of the subject antibodies to oncogenic E6 proteins.

In some embodiments, a cytometric bead array (CBA) may be used to detect binding of the subject antibodies to oncogenic E6 proteins. A Cytometric Bead Array (CBA), commonly referred to as a multiplexed bead assay, is a series of spectrally discrete particles that can be used to capture and quantitate soluble analytes, for example, an E6 protein. The analyte is then measured by detection of a fluorescence-based emission and flow cytometric analysis. The basic “sandwich assay” schema and protocols for the CBA are available at BD Biosciences. The CBA generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve.

In some embodiments, an immuno-lateral flow assay may be used to detect binding of the subject antibodies to oncogenic E6 proteins in a sample. In some embodiments, oncogenic HPV E6 is separated on a test strip. For example, oncogenic HPV E6 may be detected using a detectably labeled antibody of the present invention that binds oncogenic HPV E6. Oncogenic HPV E6 may be quantitated using a reflectance spectrophotometer, or by eye, for example. Methods and compositions for analyte detection are disclosed in US Patent Application Publication No. US20080199851, which is herein incorporated by reference in its entirety. In some embodiments, the capture antibody of the present invention may be fused or bound to another moiety including but not limited to all oligonucleotide, avidin, streptavidin, pyranosyl RNA (pRNA), aptamer or a combination thereof. The capture antibody or PDZ domain polypeptide may or may not be immobilized to a substrate.

In some embodiment, E6 protein from one oncogenic HPV strain is present and detected on the test strip using the antibody composition and the method of the present invention. In other embodiments, the antibody composition and the method of the present invention allows for detection of E6 proteins from at least two oncogenic HPV strains in a sample, for example, HPV16 and HPV18. In some embodiments, the antibody specifically binds to E6 proteins of at least 3, 4, 5, 6, 7, 8, 9, 10 or more different oncogenic HPV strains including but not limited to HPV 16, 18, 26, 30, 31, 34, 39, 45, 51, 52, 53, 58, 59, 66, 68, 69, 70, 73, and 82. HPV strain-specific E6 protein detection allows for a strip test for detecting the presence of E6 proteins, in which different HPV strains can be detected at distinct test lines on one test strip.

In many embodiments, oncogenic HPV E6 is separated from other proteins in a sample by applying the sample to one end of a test strip, and allowing the proteins to migrate by capillary action or lateral flow. Methods and devices for lateral flow separation, detection, and quantitation are known in the art. See, e.g., U.S. Pat. Nos. 5,569,608; 6,297,020; and 6,403,383. In these embodiments, a test strip comprises, in order from proximal end to distal end, a region for loading the sample (the sample-loading region) and a test region containing a capture antibody, which can be an antibody of the present invention. The sample is loaded on to the sample-loading region, and the proximal end of the test strip is placed in a buffer. Oncogenic E6 protein is captured by the bound antibody in the first test region. Detection of the captured oncogenic E6 protein is carried out as described below. For example, detection of captured E6 proteins is carried out using a detector anti-E6 antibody. The subject antibody may be used as a detector antibody in a lateral flow assay. The detector anti-E6 antibody may be detectably labeled. In some embodiments, the detector antibody is specific for an epitope of E6 proteins that is common to all oncogenic E6 proteins, or a mixture of antibodies that can, together, bind to all oncogenic E6 proteins.

In some embodiments, an immunohistochemical assay may be used to detect binding of the subject antibodies to oncogenic E6 proteins in a sample, for example, a histological sample. In some embodiments, the assay may be slide based detection of E6 proteins. Immunohistochemistry or IHC refers to the process of locating proteins in cells of a tissue section exploiting the principle of antibodies binding specifically to antigens in biological tissues. Visualizing an antibody-antigen interaction can be accomplished in a number of ways. In the most common instance, an antibody is conjugated to an enzyme, such as peroxidase, that can catalyze a color-producing reaction. Alternatively, the antibody can also be tagged to a fluorophore, such as FITC, rhodamine, Texas Red or Alexa Fluor. For IHC, the sample may be either thin (about 4-40 μm) slices taken of the tissue of interest, or the whole tissue if the tissue is not very thick and is penetrable. The sample used in IHC with the subject antibody for detection of oncogenic E6 proteins may be a cervical scrape or cervical biopsy.

Method of Detecting HPV E6 Proteins in a Sample by Antibody Sandwich Assays

In one aspect, the present invention provides a method of detecting HPV E6 proteins in a sample using an antibody sandwich detection approach. In general, the method involves (a) contacting a first capture antibody which specifically binds to E6 protein of at least one strain of HPV with the sample; (b) contacting the E6 protein that is bound to the immobilized first antibody with a second antibody, which specifically binds to E6 protein of at least one strain of HPV; and (c) detecting binding of the second antibody to the E6 protein, thereby detecting the E6 protein in the sample; wherein binding of the second antibody to the E6 protein indicates the presence of at least one HPV strain in the sample.

As used herein, the terms “sandwich”, “sandwich ELISA”, “Sandwich diagnostic” and “capture ELISA” all refer to the concept of detecting a biological polypeptide with two different test agents. In some embodiments, the sandwich assay comprises two antibodies that bind specifically to an HPV protein, for example, the E6 protein of at least one HPV strain. The two antibodies can bind to the same epitope on E6 or two different epitopes of E6 protein. For example, one anti-E6 antibody can be attached to a solid support. Test sample could be passed over the surface and the first anti-E6 antibody can bind its cognate E6 protein. An antibody with detection reagent can then be used to determine whether a specific HPV protein, for example, E6 protein had bound the first anti-E6 antibody. The term “specific binding” refers to binding between two molecules, for example, a ligand and a receptor, characterized by the ability of a molecule (ligand) to associate with another specific molecule (receptor) even in the presence of many other diverse molecules, i.e., to show preferential binding of one molecule for another in a heterogeneous mixture of molecules. Specific binding of a ligand to a receptor is also evidenced by reduced binding of a detectably labeled ligand to the receptor in the presence of excess unlabeled ligand (i.e., a binding competition assay).

For two-stage or sandwich approaches, the first antibody can bind to E6 oncoproteins at a location or epitope on the E6 protein that does not reduce the availability of the second antibody to bind to the same E6 protein. The sandwich method can improve the signal to noise ratio for a diagnostic by reducing background signal and amplifying appropriate signals. Antibodies can be generated that specifically recognize the diagnostic protein.

Preferred embodiments of the antibody sandwich assays are described in detail herein. However, it will be appreciated by ordinarily skilled practitioners that these assays can be modified in numerous ways while remaining useful for the purposes of the present invention. In some embodiments, the capture antibody is immobilized on a solid surface. The substrate to which the antibody is bound may in any of a variety of forms, e.g., a microtiter dish, a test tube, a dipstick, a microcentrifuge tube, a bead, a spinnable disk, a permeable or semi-permeable membrane, and the like. Suitable materials include glass, plastic (e.g., polyethylene, PVC, polypropylene, polystyrene, cellulose, nitrocellulose, and the like), protein, paper, carbohydrate, lipid monolayer or supported lipid bilayer, films and other solid supports. Other materials that may be employed include ceramics, metals, metalloids, semiconductive materials, cements and the like.

In some embodiments, the capture antibodies are organized as an array. The term “array,” as used herein, refers to an ordered arrangement of immobilized proteins, in which particular different proteins (i.e., recognizing different HPV proteins or different epitopes of an HPV protein, for example, E6 protein) are located at different predetermined sites on the substrate. Because the location of particular antibodies on the array is known, binding at that location can be correlated with binding to the antigen situated at that location. Immobilization of antibodies on beads (individually or in groups) is another particularly useful approach. In one embodiment, individual antibodies are immobilized on beads. In one embodiment, mixtures of distinguishable beads are used. Distinguishable beads are beads that can be separated from each other on the basis of a property such as size, magnetic property, color (e.g., using FACS) or affinity tag (e.g., a bead coated with protein A can be separated from a bead not coated with protein A by using IgG affinity methods). Binding to particular HPV protein may be determined.

When antibody-mediated immobilization methods are used, glass and plastic are especially useful substrates. The substrates may be printed with a hydrophobic (e.g., Teflon) mask to form wells. Preprinted glass slides with 3, 10 and 21 wells per 14.5 cm² slide “working area” are available from, e.g., SPI Supplies, West Chester, Pa.; also see U.S. Pat. No. 4,011,350). In certain applications, a large format (12.4 cm×8.3 cm) glass slide is printed in a 96 well format is used; this format facilitates the use of automated liquid handling equipment and utilization of 96 well format plate readers of various types (fluorescent, colorimetric, scintillation). However, higher densities maybe used (e.g., more than 10 or 100 polypeptides per cm²). See, e.g., MacBeath et al, 2000, Science 289:1760-63. Typically, antibodies are bound to substrates (e.g., glass substrates) by adsorption. Suitable adsorption conditions are well known in the art and include incubation of 0.5-50 ug/ml (e.g., 10 ug/ml) mAb in buffer (e.g., PBS, or 50 to 300 mM Tris, MOPS, HEPES, PIPES, acetate buffers, pHs 6.5 to 8, at 4° C.) to 37° C. and from 1 hr to more than 24 hours. Proteins may be covalently bound or noncovalently attached through nonspecific bonding. If covalent bonding between the fusion protein and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

Oncogenic E6 proteins can also be detected by their ability to bind to PDZ domains. Thus, in some embodiments, the method of the present invention, i.e. method of detecting oncogenic E6 protein using anti-E6 antibody sandwich assay, is compared with detecting oncogenic E6 proteins with a PDZ domain polypeptide, which involves contacting a sample containing or potentially containing an oncogenic HPV E6 protein with a PDZ domain polypeptide and detecting any binding of the oncogenic HPV E6 protein in the sample to the PDZ domain polypeptide, wherein the binding indicates the presence of an oncogenic HPV E6 protein in the sample, and thus the presence of an oncogenic HPV strain. In some embodiments, the method of the present invention enhances the signal-to-noise ratio of detecting an oncogenic E6 protein by about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 fold or more, as compared to using a PDZ domain polypeptide to detect the E6 protein. In some embodiments, the method has higher specificity of detecting an oncogenic HPV E6 protein as compared to using a PDZ domain containing polypeptide to detect the E6 protein. The method may also have higher sensitivity of detecting an oncogenic HPV E6 protein as compared to using a PDZ domain containing polypeptide to detect the E6 protein. Sensitivity can be measured by the number of E6 molecules that can be detected by the subject antibody in a given volume of a sample. Sensitivity can also be measured by the number of HPV infected cells per given volume of a sample. The In other embodiments, the method results in a lower false positive rate of erroneously detecting an oncogenic HPV E6 protein as compared to using a PDZ domain containing polypeptide to detect the E6 protein. The false positive rate of detecting E6 protein of an oncogenic HPV strain in a sample using the method of the present invention may be about 10%, 9%, 8%, 7, %, 6%, 5%, 4.5%, 4%, 3.5%, 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1% or less.

In some embodiments, the method of the present invention utilizes an enzyme linked immunosorbent assay (ELISA) to detect the presence of oncogenic E6 proteins bound to the antibody composition of the present invention. ELISA is a well known technique in the art. Briefly, ELISAs involve preparing antigen, coating the well of a 96 well multiwell plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art.

The binding affinity of an antibody to an antigen, for example, E6 protein, and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

In certain embodiments, however, the assay is an antigen capture assay, and an array or microarray of antibodies may be employed for this purpose to capture E6 proteins of more than one oncogenic HPV strains. Methods for making and using microarrays of polypeptides are known in the art (see e.g. U.S. Pat. Nos. 6,372,483, 6,352,842, 6,346,416 and 6,242,266).

### Assay Variations

As discussed supra, it will be appreciated that many of the steps in the above-described assays can be varied, for example, various substrates can be used for binding the antibodies; antibodies recognizing different E6 epitopes can be used; different labels for detecting antibody-E6 interactions can be employed; and different ways of detection can be used.

The antibody-sandwich detection assays can employ a variety of surfaces to bind the antibodies or antibody fragments. For example, a surface can be an “assay plate” which is formed from a material (e.g. polystyrene) which optimizes adherence of protein thereto. Generally, the individual wells of the assay plate will have a high surface area to volume ratio and therefore a suitable shape is a flat bottom well (where the proteins of the assays are adherent). Other surfaces include, but are not limited to, polystyrene or glass beads, polystyrene or glass slides, papers, dipsticks, plastics, films and the like.

For example, the assay plate can be a “microtiter” plate. The term “microtiter” plate when used herein refers to a multiwell assay plate, e.g., having between about 30 to 200 individual wells, usually 96 wells. Alternatively, high-density arrays can be used. Often, the individual wells of the microtiter plate will hold a maximum volume of about 250 ul. Conveniently, the assay plate is a 96 well polystyrene plate (such as that sold by Becton Dickinson Labware, Lincoln Park, N.J.), which allows for automation and high throughput screening. Other surfaces include polystyrene microtiter ELISA plates such as that sold by Nunc Maxisorp, Inter Med, Denmark. Often, about 50 ul to 300 ul, more preferably 100 ul to 200 ul, of an aqueous sample comprising buffers suspended therein will be added to each well of the assay plate.

The detectable labels of the invention can be any detectable compound or composition which is conjugated directly or indirectly with a molecule, for example, the second anti-E6 antibody of the present invention. The label can be detectable by itself (e.g., radioisotope labels or fluorescent labels) or, in the case of an enzymatic label, can catalyze a chemical alteration of a substrate compound or composition which is detectable. The preferred label is an enzymatic one which catalyzes a color change of a non-radioactive color reagent.

Direct labels include but are not limited to radioisotopes (e.g., ¹²⁵I; ³⁵S, and the like); enzymes whose products are detectable (e.g., luciferase, β-galactosidase, horse radish peroxidase, and the like); fluorescent labels (e.g., fluorescein isothiocyanate, rhodamine, phycoerythrin, and the like); fluorescence emitting metals, e.g., ¹⁵²Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin; fluorescent proteins; and the like. Fluorescent proteins include, but are not limited to, a green fluorescent protein (GFP), including, but not limited to, a “humanized” version of a GFP, e.g., wherein codons of the naturally-occurring nucleotide sequence are changed to more closely match human codon bias; a GFP derived from Aequoria victoria or a derivative thereof, e.g., a “humanized” derivative such as Enhanced GFP, which are available commercially, e.g., from Clontech, Inc.; a GFP from another species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; and the like.

Sometimes, the label is indirectly conjugated with the antibody. One of skill is aware of various techniques for direct and indirect conjugation. For example, the antibody can be conjugated with biotin and any of the categories of labels mentioned above can be conjugated with avidin, or vice versa (see also “A” and “G” assay above). Biotin binds selectively to avidin and thus, the label can be conjugated with the antibody in this indirect manner. See, Ausubel, supra, for a review of techniques involving biotin-avidin conjugation and similar assays. Alternatively, to achieve indirect conjugation of the label with the antibody, the antibody is conjugated with a small hapten (e.g. digoxin) and one of the different types of labels mentioned above is conjugated with an anti-hapten antibody (e.g. anti-digoxin antibody). Thus, indirect conjugation of the label with the antibody can be achieved.

Assay variations can include different washing steps. By “washing” is meant exposing the solid phase to an aqueous solution (usually a buffer or cell culture media) in such a way that unbound material (e.g., non-adhering cells, non-adhering capture agent, unbound ligand, receptor, receptor construct, cell lysate, or HRP antibody) is removed therefrom. To reduce background noise, it is convenient to include a detergent (e.g., Triton X) in the washing solution. Usually, the aqueous washing solution is decanted from the wells of the assay plate following washing. Conveniently, washing can be achieved using an automated washing device. Sometimes, several washing steps (e.g., between about 1 to 10 washing steps) can be required.

Various buffers can also be used in the sandwich detection assays of the present invention. For example, various blocking buffers can be used to reduce assay background. The term “blocking buffer” refers to an aqueous, pH buffered solution containing at least one blocking compound which is able to bind to exposed surfaces of the substrate which are not coated with a PL or PDZ-containing protein. The blocking compound is normally a protein such as bovine serum albumin (BSA), gelatin, casein or milk powder and does not cross-react with any of the reagents in the assay. The block buffer is generally provided at a pH between about 7 to 7.5 and suitable buffering agents include phosphate and TRIS.

Various enzyme-substrate combinations can also be utilized in detecting the first antibody-E6-second antibody sandwich interactions. Examples of enzyme-substrate combinations include but are not limited to, for example:

(i) Horseradish peroxidase (HRP or HRPO) with hydrogen peroxidase as a substrate, wherein the hydrogen peroxidase oxidizes a dye precursor (e.g. orthophenylene diamine [OPD] or 3,3′,5,5′-tetramethyl benzidine hydrochloride [TMB]) (as described above).

(ii) alkaline phosphatase (AP) with para-Nitrophenyl phosphate as chromogenic substrate.

(iii) Beta-D-galactosidase (Beta D-Gal) with a chromogenic substrate (e.g. p-nitrophenyl-Beta-D-galactosidase) or fluorogenic substrate 4-methylumbelliferyl-Beta-D-galactosidase.

Numerous other enzyme-substrate combinations are available to those skilled in the art. For a general review of these, see U.S. Pat. Nos. 4,275,149 and 4,318,980, both of which are herein incorporated by reference in their entirety.

Systems for Detecting HPV E6 Protein

The invention provides a system for detecting the presence of a HPV E6 protein, in one example, an oncogenic HPV E6 polypeptide in a sample. In general, the system comprises a first and a second antibody for a HPV E6 polypeptide, wherein the first antibody is an immobilized capture antibody specific against E6 of at least one HPV strain and the second antibody also binds to E6 of a HPV strain, e.g. an oncogenic HPV strain, and may be labeled or part of a signal producing system for detection of E6 in a sample. The second anti-E6 antibody may recognize the same epitope or a different epitope of E6 as the first anti-E6 capture antibody.

In certain embodiments, one of the binding partners is attached to a solid support, and the other binding partner may be labeled or part of a signal producing system. Proteins may be covalently bound or noncovalently attached through nonspecific bonding. If covalent bonding between the fusion protein and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

In some embodiments, oncogenic HPV E6 is separated on a test strip. For example, oncogenic HPV E6 may be detected using a detectably labeled second antibody of the present invention that binds oncogenic HPV E6. Oncogenic HPV E6 may be quantitated using a reflectance spectrophotometer, or by eye, for example. Methods and compositions for analyte detection are disclosed in US Patent Application Publication No. US20080199851, which is herein incorporated by reference in its entirety. In some embodiments, the capture antibody of the present invention may be fused or bound to another moiety including but not limited to all oligonucleotide, avidin, streptavidin, pyranosyl RNA (pRNA), aptamer or a combination thereof. The capture antibody of the present invention itself may or may not be immobilized to a substrate.

As used herein, test strip substrate refers to the material to which a capture moiety is linked using conventional methods in the art. A variety of materials can be used as the substrate, including any material that can act as a support for attachment of the molecules of interest. Such materials are known to those of skill in this art and include, but are not limited to, organic or inorganic polymers, natural and synthetic polymers, including, but not limited to, agarose, cellulose, nitrocellulose, cellulose acetate, other cellulose derivatives, dextran, dextran-derivatives and dextran co-polymers, other polysaccharides, glass, silica gels, gelatin, polyvinyl pyrrolidone (PVP), rayon, nylon, polyethylene, polypropylene, polybutylene, polycarbonate, polyesters, polyamides, vinyl polymers, polyvinylalcohols, polystyrene and polystyrene copolymers, polystyrene cross-linked with divinylbenzene or the like, acrylic resins, acrylates and acrylic acids, acrylamides, polyacrylamide, polyacrylamide blends, co-polymers of vinyl and acrylamide, methacrylates, methacrylate derivatives and co-polymers, other polymers and co-polymers with various functional groups, latex, butyl rubber and other synthetic rubbers, silicon, glass, paper, natural sponges, insoluble protein, surfactants, red blood cells, metals, metalloids, magnetic materials, or other commercially available media or a complex material composed of a solid or semi-solid substrate coated with materials that improve the hydrophilic property of the strip substrate, for example, polystyrene, Mylar, polyethylene, polycarbonate, polypropylene, polybutylene, metals such as aluminum, copper, tin or mixtures of metals coated with dextran, detergents, salts, PVP and/or treated with electrostatic or plasma discharge to add charge to the surface thus imparting a hydrophilic property to the surface.

In one embodiment, the lateral flow membrane is comprised of a porous material such as high density polyethylene sheet material manufactured by Porex Technologies Corp. of Fairburn, Ga., USA. The sheet material has an open pore structure with a typical density, at 40% void volume, of 0.57 gm/cc and an average pore diameter of 1 to 250 micrometers, the average generally being from 3 to 100 micrometers. In another embodiment, the label zone is comprised of a porous material such as a nonwoven spunlaced acrylic fiber (similar to the sample receiving zone), e.g., New Merge or HDK material. Often, the porous material may be backed by, or laminated upon, a generally water impervious layer, e.g., Mylar. When employed, the backing is generally fastened to the matrix by an adhesive (e.g., 3M 444 double-sided adhesive tape). Typically, a water impervious backing is used for membranes of low thickness. A wide variety of polymers may be used provided that they do not bind nonspecifically to the assay components and do not interfere with flow of the fluid sample. Illustrative polymers include polyethylene, polypropylene, polystyrene and the like. On occasion, the matrix may be self-supporting. Other membranes amenable to non-bibulous flow, such as polyvinyl chloride, polyvinyl acetate, copolymers of vinyl acetate and vinyl chloride, polyamide, polycarbonate, polystyrene, and the like, can also be used. In yet another embodiment the lateral flow membrane is comprised of a material such as untreated paper, cellulose blends, nitrocellulose, polyester, an acrylonitrile copolymer, and the like. The label zone may be constructed to provide either bibulous or non-bibulous flow, frequently the flow type is similar or identical to that provided in at least a portion of the sample receiving zone. In a frequent embodiment, the label zone is comprised of a nonwoven fabric such as Rayon or glass fiber. Other label zone materials suitable for use by the present invention include those chromatographic materials disclosed in U.S. Pat. No. 5,075,078, which is herein incorporated by reference.

In a frequent embodiment, the test strip substrate is treated with a solution that includes material-blocking and label-stabilizing agents. Blocking agents include bovine serum albumin (BSA), methylated BSA, casein, acid or base hydrolyzed casein, nonfat dry milk, fish gelatin, or similar. Stabilizing agents are readily available and well known in the art, and may be used, for example, to stabilize labeled reagents. In some embodiments, the upstream compartment containing a solution 307 can comprise multiple ampules, which can be selectively punctured or broken to release their contents. Therefore, in one embodiment, blocking reagents are contained in one ampule which is utilized to pre-treat (e.g., “block”) the test strip (i.e., lateral flow membrane), while the additional ampule is reserved for washing the sample through the test strip.

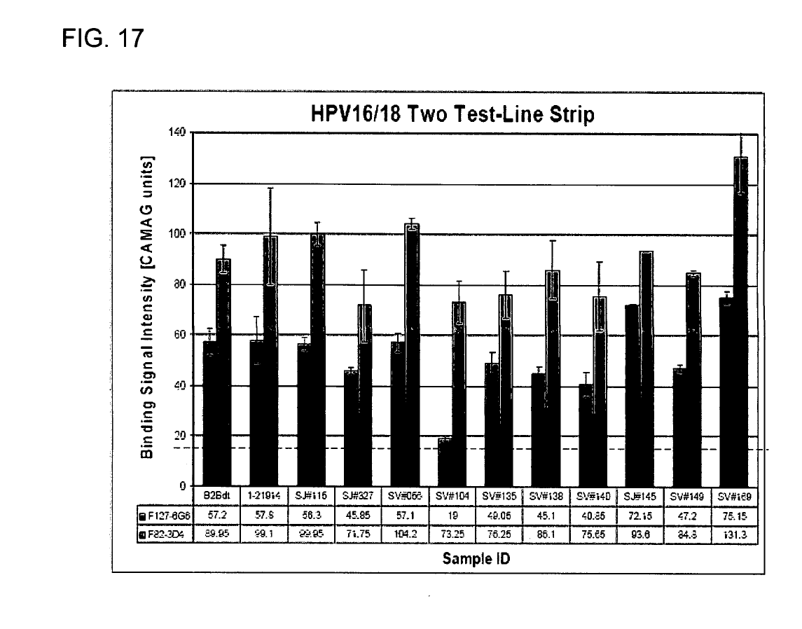

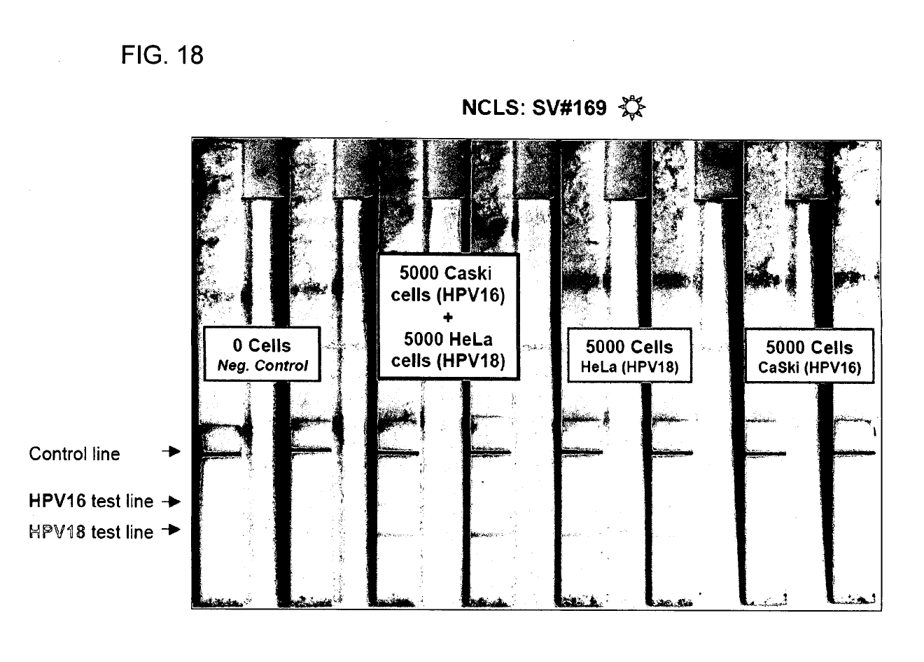

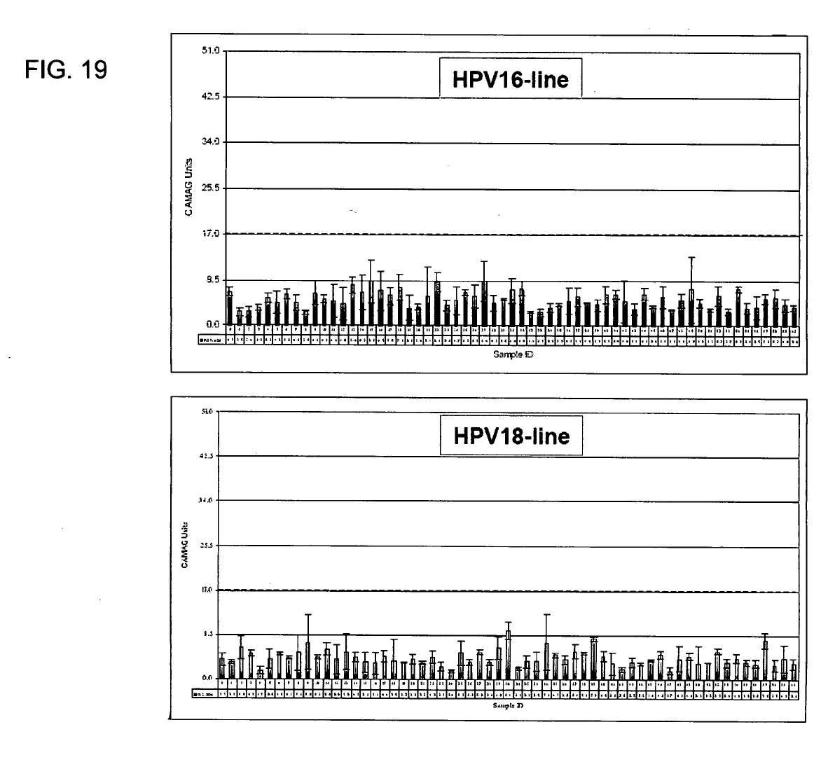

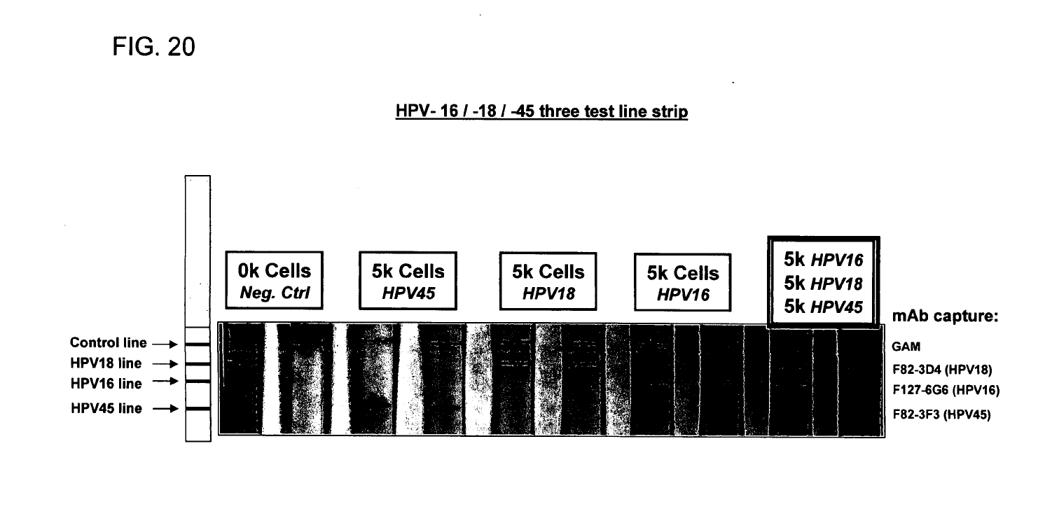

In some embodiment, E6 protein from one oncogenic HPV strain is present and detected on the test strip using the antibody composition and the method of the present invention. In other embodiments, the antibody composition and the method of the present invention allows for detection of E6 proteins from more than one oncogenic HPV strains in a sample, for example, HPV16 and HPV18. HPV strain-specific E6 protein detection allows for a strip test for detecting the presence of E6 proteins, in which different HPV strains can be detected at distinct test lines on one test strip. For example, E6 proteins from HPV16 and HPV18 are detected as two distinct lines on a two test-line strip using anti-E6 HPV16+HPV18 antibody detector cocktail of the present invention as shown in FIGS. 17 and 18. In another example, E6 proteins from HPV16, HPV18 and HPV45 are detected as three distinct lines on a three test-line strip using anti-E6 HPV16+HPV18+HPV45 antibody detector cocktail of the present invention as shown in FIG. 20. Such Strip test approach utilizing the antibody sandwich detection method of the present invention results in a low false positive rate of detecting oncogenic E6 proteins in a sample. In some embodiments, the false positive rate of detecting E6 is 0 out of 60 biological samples tested (FIG. 19). The false positive rate may be less than about 10%, 9%, 8%, 7, %, 6%, 5%, 4.5%, 4%, 3.5%, 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1% or less.

In many embodiments, oncogenic HPV E6 is separated from other proteins in a sample by applying the sample to one end of a test strip, and allowing the proteins to migrate by capillary action or lateral flow. Methods and devices for lateral flow separation, detection, and quantitation are known in the art. See, e.g., U.S. Pat. Nos. 5,569,608; 6,297,020; and 6,403,383. In these embodiments, a test strip comprises, in order from proximal end to distal end, a region for loading the sample (the sample-loading region) and a test region containing the first anti-E6 antibody, i.e. the capture antibody of the present composition. The sample is loaded on to the sample-loading region, and the proximal end of the test strip is placed in a buffer. Oncogenic E6 protein is captured by the bound antibody in the first test region. Detection of the captured oncogenic E6 protein is carried out as described below. For example, detection of captured E6 proteins is carried out using a second anti-E6 antibody of the present composition. The second anti-E6 antibody may be detectably labeled. In some embodiments, the second antibody is specific for an epitope of E6 proteins that is common to all oncogenic E6 proteins, or a mixture of antibodies that can, together, bind to all oncogenic E6 proteins.

Identification of Complementary Antibody Pair

In one aspect, the present invention provides a method of screening antibodies for antibody pairs that bind to E6 protein of a HPV strain with enhanced sensitivity as compared to an individual antibody specific against the E6 protein, the method comprising: (a) contacting a first antibody of the antibody pair with the E6 protein; (b) contacting the E6 protein that is bound to the first antibody with a second antibody of the antibody pair; (c) detecting binding of the second antibody to the E6 protein that is bound to the first antibody of the antibody pair; and selecting the antibody pair that has a higher signal-to-noise ratio as compared to the unpaired individual antibodies; wherein a higher signal-to-noise ratio indicates an enhanced sensitivity of E6 protein detection.

In one aspect, the present invention discloses the development of an immunoassay to detect antibody pairs that bind target antigen, for example, E6 protein, with high affinity. In general, one antibody is in contact with the target antigen, for example, E6 protein, and the ability of the second antibody to bind the E6 protein that is bound to the first antibody is determined. The first antibody itself may or may not be immobilized to a substrate. In one example, the first antibody is immobilized to a solid substrate. In another example, the first antibody is fused or bound to another moiety including but not limited to all oligonucleotide, avidin, streptavidin, pyranosyl RNA (pRNA), aptamer or a combination thereof. Methods and compositions for analyte detection are disclosed in US Patent Application Publication No. US20080199851, which is herein incorporated by reference in its entirety. Various assay formats known in the art can be used to identify antibody pairs that bind to E6 protein with higher affinity and higher sensitivity than the individual antibodies alone using the method of the present invention. These assays include but are not limited to solid-phase ELISA immunoassays, immunoprecipitation, Biacore, and Western blot assays. These assays, which are disclosed herein, can be readily used to screen for hundreds to thousands of potential antibody-E6 interactions in a short period of time. Thus these assays can be used to identify yet more novel synergistic antibodies that interact with E6 proteins with high affinity.

In one example, for illustrative purposes, ELISA plate(s) are coated with sheep anti-mouse IgG Fc γ, washed, and blocked; then incubated with the first antibody, washed and blocked. The antigen, for example, E6 protein, is introduced to the plate, incubated and washed. The second antibody is then incubated with Alkaline Phosphatase labeled F(ab′)₂ and subsequently blocked with murine IgG prior to introduction to the plate containing the antigen, for example E6, bound to the first antibody. The presence of an immunological sandwich (antibody 1 bound to antigen; the antigen bound to antibody 2) is detected colorimetrically with PNPP by reading absorbance at 405 nm. Signal to Noise (S/N) ratios are calculated to identify those synergistic antibodies producing the highest signals, thus illustrating an antibody pair. The identified antibody pairs are successfully implemented in various immunoassay formats, for example, lateral flow formats.

The antibodies of the invention may be screened for immunospecific binding by any method known in the art. The immunoassays which can be used in identifying antibody pairs, for example, an antibody pair of the present invention include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, and cellular immunostaining (fixed or native) assays to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

Antibodies of the invention may be screened using immunocytochemisty methods on cells (e.g., mammalian cells, such as CHO cells) transfected with a vector enabling the expression of an antigen or with vector alone using techniques commonly known in the art. Antibodies that bind antigen transfected cells, but not vector-only transfected cells, are antigen specific.

In certain embodiments, however, the assay is an antigen capture assay, and an array or microarray of antibodies may be employed for this purpose. Methods for making and using microarrays of polypeptides are known in the art (see e.g. U.S. Pat. Nos. 6,372,483, 6,352,842, 6,346,416 and 6,242,266).

Various known methods can be used to purify the identified antibody pairs. These methods include but are not limited to immunofiltration or affinity purification of antibodies. Briefly, immunofiltration refers to purification of antibodies by mixing with specific antigen. The antigen is then removed from the antibody by treatment with soluble carriers. Hybridoma cells whose supernatants give the desired activity are selected for cloning. In one example, cells are cloned by limiting dilution in a 96-well flat bottom plate. Purification of antibodies from tissue culture supernatants can be performed by protein G and A affinity chromatography (Amersham). The isotype of the antibodies can be determined using Cytometric bead array. Antibody purification methods and protocols are well established in the art and are within the knowledge of one skilled in the art.

Sample Collection

Biological samples to be analyzed using the methods of the invention may be obtained from any mammal, e.g., a human or a non-human animal model of HPV. In many embodiments, the biological sample is obtained from a living subject.

In some embodiments, the subject from whom the sample is obtained is apparently healthy, where the analysis is performed as a part of routine screening. In other embodiments, the subject is one who is susceptible to HPV, (e.g., as determined by family history; exposure to certain environmental factors; etc.). In other embodiments, the subject has symptoms of HPV (e.g., cervical warts, or the like). In other embodiments, the subject has been provisionally diagnosed as having HPV (e.g. as determined by other tests based on e.g., PCR).

The biological sample may be derived from any tissue, organ or group of cells of the subject. In some embodiments a cervical scrape, biopsy, or lavage is obtained from a subject. In other embodiments, the sample is a blood or urine sample. In still other embodiments, the sample is a histological sample.

In some embodiments, the biological sample is processed, e.g., to remove certain components that may interfere with an assay method of the invention, using methods that are standard in the art. In some embodiments, the biological sample is processed to enrich for proteins, e.g., by salt precipitation, and the like. In certain embodiments, the sample is processed in the presence protease inhibitor to inhibit degradation of the E6 protein.

In the assay methods of the invention, in some embodiments, the level of E6 protein in a sample may be quantified and/or compared to controls. Suitable control samples are from individuals known to be healthy, e.g., individuals known not to have HPV. Control samples can be from individuals genetically related to the subject being tested, but can also be from genetically unrelated individuals. A suitable control sample also includes a sample from an individual taken at a time point earlier than the time point at which the test sample is taken, e.g., a biological sample taken from the individual prior to exhibiting possible symptoms of HPV.

In certain embodiments, a sample is contacted to a solid support-bound antibody or PDZ domain polypeptide under conditions suitable for binding of the antibody or the PDZ domain polypeptide to E6 proteins in the sample, and after separation of unbound sample proteins from the bound proteins, the bound proteins are detected using the subject antibody using known methods.

Diagnosing the presence of pathogens requires collection of samples appropriate to the organism. For detection of HPV E6 proteins, one would collect tissue for testing from the cervix, penis, anus, or throat using a scrape, swab or biopsy technique. For diagnosis of bloodborne pathogens such as HIV, collection of blood through standard means would be most appropriate. Diagnosis of fungal or viral infections that may have caused skin lesions would require the collection of a sample from the affected area.

This invention is not intended to cover sampling devices. However, it should be noted that since the invention is predicated on the detection of E6 proteins, appropriate care must be taken to collect a sufficient amount of sample to detect pathogen proteins and to maintain the integrity of proteins in the sample. The amount of sample to collect should be determined empirically for each diagnostic test. Factors in the decision may include, but not be limited to, the stage at which detection is desired, the amount of pathogen per unit sample, the amount of diagnostic protein per unit per unit sample, availability of diagnostic epitopes and the stability of diagnostic epitopes.

Exemplary collection devices for cervical tissue include, but are not limited to, those described in U.S. Pat. Nos. 6,241,687, 6,352,513, 6,336,905, 6,115,990 and 6,346,086. These collection devices would facilitate the collection of cervical tissue for the diagnosis of oncogenic human papillomavirus infection. These devices are predominantly collection of cervical cells or tissues through scraping; alternatively, one could use standard biopsy methods to collect samples from any tissues to be examined.

Although the diagnostic method disclosed in this application is directed at the detection of E6 proteins, sample collection need not be limited to collection of proteins. One could alternatively collect RNA from tissue samples, use an in vitro translation kit to produce protein from collected templates, and then assay using methods disclosed herein. In a similar manner, DNA could be collected from test samples, specific primers for oncogenic E6 and E7 proteins could be used to either amplify the DNA content (using a DNA polymerase) or transcribe and translate the sample into proteins that could be tested with methods disclosed herein.

“Subject”, “individual,” “host” and “patient” are used interchangeably herein, to refer to any animal, e.g., mammal, human or non-human. Generally, the subject is a mammalian subject. Exemplary subjects include, but are not necessarily limited to, humans, non-human primates, mice, rats, cattle, sheep, goats, pigs, dogs, cats, birds, deer, elk, rabbit, reindeer, deer, and horses, with humans being of particular interest.

Specificity and Sensitivity

In some embodiments, the term “specific binding” or “specificity” refers to the ability of an antibody or a combination of antibodies to preferentially bind to a particular analyte or component that is present in a homogeneous mixture of different analytes or components in a biological sample. The term “analyte” is used herein interchangeably and refers to a known or unknown component of a sample. Typically, a specific binding interaction will discriminate between desirable and undesirable analytes in a sample, typically more than about 10 to 100-fold or more (e.g., more than about 1000- or 10.000-fold). Typically, the affinity between a capture agent e.g. an antibody or a polypeptide and an analyte in a sample when they are specifically bound in an antibody/antigen complex is at least 10⁻⁷, at least 10⁻⁸ M, at least 10⁻⁹ M, at least 10⁻¹⁰ M, usually up to about 10⁻¹¹ M. In some embodiments, specificity refers to the proportion of people without HPV who have a negative test result.

In some embodiments, the HPV E6 protein detection method of the present invention using the subject antibodies has a high specificity of detecting oncogenic E6 proteins. In some embodiments, the specificity of the HPV detection method of the invention is about 85%, 90%, 91%, 92%, 93%, 94%, 95%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or more than 99.9%. In some embodiments, the rate of erroneously detecting an E6 protein of a high-risk HPV strain when such HPV strain is in fact absent in a sample, i.e. false positive rate, is about 10%, 9%, 8%, 7%, 6%, 5%, 4.5%, 4%, 3.5%, 3%, 2.5%, 2%, 1.5%, 1%, 0.9%, 0.8%, 0.7%, 0.6%, 0.5%, 0.4%, 0.3%, 0.2%, 0.1%, or less than 0.1%.

In some embodiments, the specificity of detecting E6 protein using the method of the present invention is increased as compared to using a PDZ domain containing polypeptide that bind to E6 protein. In one embodiment of the invention, the binding of E6 to anti-E6 antibodies of the present invention or a plurality of PDZ proteins is determined. Using this method, it is possible to compare an anti-E6 antibody with a PDZ domain polypeptide bound with particular specificity by the E6 protein. In some embodiments, the specificity of detecting oncogenic E6 protein using the composition and the method of the present invention is increased to about 85%, 90%, 91%, 92%, 93%, 94%, 95%, 96%, 96.5%, 97%, 97.5%, 98%, 98.5%, 99%, 99.1%, 99.2%, 99.3%, 99.4%, 99.5%, 99.6%, 99.7%, 99.8%, 99.9% or more. As compared to using a PDZ domain containing polypeptide to detect E6 protein, the specificity of E6 detection using the antibody sandwich method of the present invention is increased by about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 500, 1,000, 10,000 fold or more.

The subject antibody binds N-terminus of oncogenic E6 proteins with high affinity. In some embodiments, the antibody binds to E6 protein with a binding affinity of less than 10⁻⁸ M, 10⁻⁹M, 10⁻¹⁰ M, 10⁻¹¹ M, or 10⁻¹² M. In some embodiments, the subject antibody binds to E6 proteins of high-risk HPV strains with at least 1.5, 1.6, 1.7, 1.8, 1.9, 2, 2.1, 2.2, 2.3, 2.4, 2.5, 2.6, 2.6, 2.8, 2.9, 3.0, 3.5, 4, 4.5, 5, 5.5, 6, 6.5, 7, 7.5, 8, 8.5, 9, 9.5, 10, 20, 30, 40, 50, 60, 70, 80, 90, 100, or more than 100 times higher affinity than the previously available antibodies that are specific for E6 proteins. The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody. Binding affinity of an antibody to an antigen may also be measured via known techniques in the art including but not limited to BiaCore analysis, which is based on surface plasmon resonance (SPR), and ELISA.

The subject antibody binds C-terminus of oncogenic E6 proteins with high affinity. For example, the antibody may bind to E6 protein with a binding affinity of less than 10⁻⁸ M, less than 10⁻⁹ M, less than 10⁻¹⁰ M, less than 10⁻¹¹ M, or less than 10⁻¹² M. In some embodiments, the subject antibody binds to E6 proteins of high-risk HPV strains with at least 1.5, 1.6, 1.7, 1.8, 1.9, 2, 2.1, 2.2, 2.3, 2.4, 2.5, 2.6, 2.6, 2.8, 2.9, 3.0, 3.5, 4, 4.5, 5, 5.5, 6, 6.5, 7, 7.5, 8, 8.5, 9, 9.5, 10, 20, 30, 40, 50, 60, 70, 80, 90, 100 or more than 100 times higher affinity than the previously available antibodies that are specific for E6 proteins. The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody. Binding affinity of an antibody to an antigen may also be measured via known techniques in the art including but not limited to BiaCore analysis, which is based on surface plasmon resonance (SPR), and ELISA.

In some embodiments, sensitivity refers to the proportion of people with HPV who have a positive test result. In other embodiments, sensitivity refers to the smallest amount of a substance, such as a protein in a sample, which a diagnostic test can detect. In some embodiments, the sensitivity of correctly detecting oncogenic E6 proteins of at least two high-risk HPV strains is about 70%, 75%, 80%, 85%, 90%, 91%, 92%, 93%, 94%, 95%, 96%, 97%, 97.5%, 98%, 98.5%, 99%, 99.1%, 99.2%, 99.3%, 99.4%, 99.5%, 99.6%, 99.7%, 99.8%, 99.9% or more than 99.9%. Sensitivity can be measured by the number of E6 molecules that can be detected by the subject antibody in a given volume of a sample, for example, # of E6 molecules/ml of sample. For example, the subject method can detect about 200-300 pg of recombinant E6 protein per ml of sample. In some embodiments, the subject method detects about 1 fg of E6 protein per dysplastic cell. Sensitivity can also be measured by the number of HPV infected cells per given volume of a sample. Understanding the sensitivity of the antibody of the present invention is essential because it helps to define the amount of tissue or cell sample that must be tested to obtain a definitive result.

Sensitivity of E6-antibody binding can be measured based on apparent affinity, which is determined based on the concentration of one molecule required to saturate the binding of a second molecule, e.g. the binding of a ligand to a receptor, in this case, binding of E6 to a subject anti-E6 antibody. In one example, a fixed concentration of an anti-E6 antibody of the present invention and increasing concentrations of a labeled E6 peptide (labeled with, for example, biotin or fluorescein) are mixed together in solution and allowed to react. Representative HPV E6 peptide amino acid sequences are disclosed herein and also in U.S. Pat. Nos. 7,312,041 and 7,399,467, which are both incorporated by reference in their entirety. In one embodiment, preferred E6 peptide concentrations are 0.1 uM, 1 uM, 10 uM, 100 uM, 1 mM. In various embodiments, appropriate reaction times can range from 10 minutes to 2 days at temperatures ranging from 4° C. to 37° C. In some embodiments, the identical reaction can also be carried out using a nonspecific antibody as a control. Antibody-E6 complexes can be separated from unbound labeled peptide using a variety of methods known in the art. For example, the complexes can be separated using high performance size-exclusion chromatography (HPSEC, gel filtration) (Rabinowitz et al., 1998, Immunity 9:699), affinity chromatography (e.g. using glutathione Sepharose beads), and affinity absorption (e.g., by binding to an anti-GST-coated plate as described supra). The antibody-E6 complex is detected based on presence of the label on the E6 peptide ligand using a variety of methods and detectors known to one of skill in the art. For example, if the label is fluorescein and the separation is achieved using HPSEC, an in-line fluorescence detector can be used. The antibody-E6 binding signal is plotted as a function of ligand concentration and the plot is fit. (e.g., by using the Kaleidagraph software package curve fitting algorithm) to the following equation, where “Signal_{[ligand]}” is the binding signal at PL peptide concentration “[ligand],” “Kd” is the apparent affinity of the binding event, and “Saturation Binding” is a constant determined by the curve fitting algorithm to optimize the fit to the experimental data:

Signal_{[ligand]}=Saturation Binding×([ligand]/([ligand+Kd])

The calculation of binding affinity itself can be determined using any suitable equation (see Cantor and Schimmel (1980) BIOPHYSICAL CHEMISTRY W H Freeman & Co., San Francisco) or software. It will be appreciated that binding assays are conveniently carried out in multiwell plates (e.g., 24-well, 96-well plates, or 384 well plates).

It will be recognized that high specificity and high sensitivity interactions between an E6-binding partner and E6 represent potentially more valuable system for detecting oncogenic HPV strains in a sample. Signal-to-noise ratio typically compares the level of a desired signal, for example, specific binding to E6 protein, and the level of background noise, for example, any unspecific binding not to E6 protein. The higher the ratio, the less obtrusive the background noise is. In some embodiments, the method of the present invention enhances the signal-to-noise ratio of detecting oncogenic E6 proteins of at least two HPV strains by about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100 fold or more, as compared to using previously available anti-E6 antibodies or a PDZ domain polypeptide to detect E6 proteins.

In some embodiments, the method of the present invention results in a lower false positive rate of erroneously detecting an oncogenic HPV E6 protein as compared to using a PDZ domain containing polypeptide to detect the E6 protein. The false positive rate for a test is the false-positive test results divided by all patients without the disease. In one example, the method of the present invention results in 0 false positive on 60 individual HPV negative cervical swab samples (FIG. 19). The false positive rate of detecting E6 protein of an oncogenic HPV strain in a sample using the method of the present invention may be about 10%, 9%, 8%, 7, %, 6%, 5%, 4.5%, 4%, 3.5%, 3%, 2.9%, 2.8%, 2.7%, 2.6%, 2.5%, 2.4%, 2.3%, 2.2%, 2.1%, 2%, 1.9%, 1.8%, 1.7%, 1.6%, 1.5%, 1.4%, 1.3%, 1.2%, 1.1%, 1%, 0.9%, 0.8%, 0.7%, 0.6%, 0.5%, 0.4%, 0.3%, 0.2%, 0.1% or less. In one embodiment, the false positive rate of detecting oncogenic E6 protein in a sample is less than 1.7%.

Kits

The present invention also includes kits for carrying out the methods of the invention. In some embodiments, a subject kit contains a subject antibody that specifically binds to oncogenic E6 proteins of at least two HPV strains. In some embodiments, the antibody is a capture antibody. In some embodiments, the antibody is a detector antibody. In some embodiments, the antibody is labeled with a detectable label. In other embodiments, a secondary labeling component, such as a detectably labeled secondary antibody, is included. In some embodiments, a subject kit further comprises a PDZ domain containing polypeptide, such as MAGI-1. In some embodiments, a subject kit further comprises a means, such as a device or a system, for isolating oncogenic HPV E6 protein from the sample. In some embodiments, a subject kit contains a capture antibody and a detection antibody of the present invention that specifically bind to E6 protein of a HPV strain. In some embodiments, the kit further contains a third antibody for binding to the second antibody for detection of the E6 protein. In some embodiments, the second antibody is labeled with a detectable label. In other embodiments, a secondary labeling component, such as a detectably labeled third antibody, is included. In some embodiments, a subject kit further comprises a means, such as a device or a system, for isolating HPV E6 protein from the sample. The kit may optionally contain protease inhibitor. In some embodiments, the diagnostic kit further contains a strip for performing the method of the present invention, i.e. on which the antibodies of the present invention bind to E6 protein in a sample. The diagnostic kit may detect E6 protein of one HPV strain or more than one HPV strain. For example, the diagnostic kit may detect oncogenic E6 proteins of HPV-16, HPV-18, HPV-45 or a combination thereof. In other embodiments, the kit contains antibodies of the present invention that specifically bind to E6 proteins of low-risk HPV strains, for example, HPV6 and HPV11. In some embodiments, the kit contains reagents for performing enzyme-linked immunosorbent assay (ELISA).

A subject kit can further include, if desired, one or more of various conventional components, such as, for example, containers with one or more buffers, detection reagents or antibodies. Printed instructions, either as inserts or as labels, indicating quantities of the components to be used and guidelines for their use, can also be included in the kit. In the present disclosure it should be understood that the specified materials and conditions are important in practicing the invention but that unspecified materials and conditions are not excluded so long as they do not prevent the benefits of the invention from being realized. Exemplary embodiments of the diagnostic methods of the invention are described above in detail.

In a subject kit, the oncogenic E6 protein detection reaction may be performed using an aqueous or solid substrate, where the kit may comprise reagents for use with several separation and detection platforms such as test strips, sandwich assays, etc. Kits may also include components for conducting western blots (e.g., pre-made gels, membranes, transfer systems, etc.); components for carrying out ELISAs (e.g., 96-well plates); components for carrying out immunoprecipitation (e.g. protein A); columns, especially spin columns, for affinity or size separation of oncogenic E6 protein from a sample (e.g. gel filtration columns, PDZ domain polypeptide columns, size exclusion columns, membrane cut-off spin columns etc.).

Subject kits may also contain control samples containing oncogenic or non-oncogenic E6 proteins, and/or a dilution series of oncogenic E6 proteins, where the dilution series represents a range of appropriate standards with which a user of the kit can compare their results and estimate the level of oncogenic E6 proteins in their sample. Such a dilution series may provide an estimation of the progression of any cancer in a patient. Fluorescence, color, or autoradiological film development results may also be compared to standard curves of fluorescence, color or film density provided by the kit.

In addition to above-mentioned components, the subject kits typically further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the interne, are provided. An example of this embodiment is a kit that includes a web address where the instructions can be viewed and/or from which the instructions can be downloaded. As with the instructions, this means for obtaining the instructions is recorded on a suitable substrate.

Utility

An effective diagnostic to detect high-risk HPV infection requires a “pan” antibody that is able to detect the most clinically relevant high-risk HPV types. Currently, HPV DNA tests lack the specificity required to decrease the number of “non-cancer progressing” women referred for colposcopy. In contrast, HPV RNA tests lack the clinical sensitivity for use as a primary screen. A protein based HPV E6 test, which is embodied in the present invention, may thus have the advantage of possessing the sensitivity and specificity needed for a cancer screen, preferably cervical cancer screen.

HPV E6 initiates oncogenesis by binding to tumor suppressors and oncogenes via the PDZ binding domain. It also allows specific separation of HPV strains, useful for diagnostic and therapeutic development. The method of the present invention offers potential for antibodies to complement binding of antibodies that are specific for the carboxyl-terminal (C-terminal) region of E6 antibodies and avoid masking of the PDZ binding domain. In addition, the antibody of the present invention can specifically recognize HPV16 and HPV18 only, which allows for pre-screening of individuals prior to receiving vaccination with Gardasil, which is a cervical cancer vaccine against 4 types of HPV, i.e. HPV6, 11, 16, and 18, or other HPV-related vaccines.

Some of the advantages of developing antibodies to the PDZ binding motif at the C-terminal region of the oncogenic E6 proteins of high-risk HPV strains include but are not limited to: first, it confers high specificity of HPV detection through the binding to the PDZ domain motif only found in high-risk HPV types. Thus, it may allow capture of specific HPV strains, such as HPV16 and HPV18, and also allow specific separation of the oncogenic HPV strains, useful for diagnostic and therapeutic development. Second, the method of the present invention offers potential for antibodies to complement binding of antibodies that are specific for the amino-terminal (N-terminal) region of E6 antibodies. Third, it allows extraction of aggregated and insoluble HPV E6 protein in Urea with the potential to gain analytical sensitivity in assays tolerating high concentrations of Urea. Fourth, it avoids reliance on the specific binding of PDZ domain containing polypeptides (i.e. hDlg, MAGI-1, MUPP1) and obviates the need to decrease binding of these PDZ domain containing polypeptides to their endogenous ligands in cell lysates, thereby resulting in less background noise. Finally, the antibody of the present invention can specifically recognize HPV16 and HPV18 only, which allows for pre-screening of individuals prior to receiving vaccination with Gardasil, which is a cervical cancer vaccine against 4 types of HPV, i.e. HPV6, 11, 16, and 18, or other HPV-related vaccines.

Furthermore, the method of the present invention may also be used as part of a test to detect low-risk HPV types in pregnant women as there is a risk of developing perinatal infection of the fetus with low-risk HPV types, such as HPV6 and HPV11.

The antibody composition and methods of the instant invention are useful for a variety of diagnostic analyses. The instant antibodies and methods are useful for diagnosing infection by an oncogenic strain of HPV in an individual; for determining the likelihood of having cancer; for determining a patient's response to treatment for HPV; for determining the severity of HPV infection in an individual; and for monitoring the progression of HPV in an individual. The antibodies and the methods of the instant invention are useful in the diagnosis of infection with an oncogenic or a non-oncogenic strain of HPV associated with cancer, including cervical, ovarian, breast, anus, penis, prostate, larynx and the buccal cavity, tonsils, nasal passage, skin, bladder, head and neck squamous-cell, occasional periungal carcinomas, as well as benign anogenital warts. The antibodies and the methods of the instant invention are useful in the diagnosis of infection with an oncogenic or a non-oncogenic strain of HPV associated with Netherton's syndrome, epidermolysis verruciformis, endometriosis, and other disorders. The antibodies and the methods of the instant invention are useful in the diagnosis of infection with an oncogenic or a non-oncogenic strain of HPV in adult women, adult men, fetuses, infants, children, and immunocompromised individuals.

In some embodiments, the antibodies of the present invention can be used for the amelioration of an HPV disease, comprising administering to a subject in need thereof an effective amount of an antibody which specifically binds an HPV E6 protein. By amelioration of an HPV disease is meant to include methods of treating, suppressing or prevent an HPV disease or symptom of an HPV disease. HPV diseases include those listed above, such as cervical cancer and warts. In some embodiments, the antibody specifically binds to carboxyl-terminus (C-terminus) of the E6 protein. In some embodiments, the antibody specifically binds to carboxyl-terminus (C-terminus) of oncognenic E6 proteins of at least two HPV strains with the sample. In some embodiments, the antibody specifically binds to E6 proteins of HPV strains 16, 18, and 45; strains 16, 18, 31, 33, 45, 52, and 58; strains 16, 18, 26, 30, 31, 39, 45, 51, 68, 69, and 82; or strains 16, 18, 26, 30, 31, 34, 39, 45, 51, 52, 53, 58, 59, 66, 68, 69, 70, 73, and 82. In some embodiments, the antibody binds to at least 2, 3, 4, 5, 6, 7, 8, 9, 10, 15, 20 or 25 different HPV strains. In some embodiments, the antibody binds to oncogenic HPV strains. In some embodiments, the antibody binds to oncogenic HPV strains and not non-oncogenic HPV strains. In some embodiments, the antibody binds to E6 protein with a binding affinity of less than 10⁻⁸ M, M, 10⁻¹⁰ M, 10⁻¹¹ M, or 10⁻¹² M. In some embodiments, the antibody is a monoclonal antibody. In some embodiments, a combination of antibodies is used, as described above, in order to ameliorate an HPV disease. In some embodiments, an antibody of the invention can be combined with another therapeutic for the treatment of an HPV disease. For example, an antibody of the invention can be combined with another anti-cancer agent for the treatment of cervical cancer. Suitable dosages for any of the above co-administered agents are those presently used and may be lowered due to the combined action (synergy) of the agent and the antibody of the present invention.

For the prevention or treatment of disease, the appropriate dosage of antibody will depend on the type of disease to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g. 0.1-20 mg/kg) of antibody is an initial candidate dosage for administration to the patient, whether, for example, by one or more separate administrations, or by continuous infusion. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs.

In some embodiments, the dosage of the antibody will be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the subject. Such doses may be administered intermittently, e.g. every week or every three weeks (e.g. such that the patient receives from about two to about twenty, e.g. about six doses, of the antibody). An initial higher loading dose, followed by one or more lower doses, may be administered. An exemplary dosing regimen comprises administering an initial loading dose of about 1-20 mg/kg, 1-10 mg/kg, 1-5 mg/kg, 3-5 mg/kg or 4 mg/kg, followed by a weekly maintenance dose of about 1-20 mg/kg, 1-10 mg/kg, 1-5 mg/kg, 2-5 mg/kg or 2 mg/kg of the antibody. However, other dosage regimens may be useful. The progress of this therapy is easily monitored by conventional techniques and assays.

In some embodiments, the antibody is administered in the dosage range of about 1.0 to 5.0 μmol, or 2.0 to 4.0 μmol of antibody per cell (such as per HPV infected cell). In some instances, a subject can be infected with 30,000 to 40,000 E6 molecules per cell. Thus, this dosing range could be used to effectively treat a subject having this level of E6 load per cell.

Alternatively, the antibody is suitably administered serially or in combination with radiological treatments—irradiation or introduction of radioactive substances—such as those referred to in UICC (Ed.), Klinische Onkologie, Springer-Verlag (1982).

Aside from administration of the antibody to the subject, the present application contemplates administration of the antibody by gene therapy. Such administration of nucleic acid encoding the antibody is encompassed by the expression “administering a therapeutically effective amount of an antibody”. See, for example, WO 1996/07321 published Mar. 14, 1996 concerning the use of gene therapy to generate intracellular antibodies.

There are two major approaches to getting the nucleic acid (optionally contained in a vector) into the subject's cells, in vivo and ex vivo. For in vivo delivery the nucleic acid is injected directly into the subject, usually at the site where the antibody is required. For ex vivo treatment, the subject's cells are removed, the nucleic acid is introduced into these isolated cells, and the modified cells are administered to the subject either directly or, for example, encapsulated within porous membranes that are implanted into the subject (see, e.g. U.S. Pat. Nos. 4,892,538 and 5,283,187). There are a variety of techniques available for introducing nucleic acids into viable cells. The techniques vary depending upon whether the nucleic acid is transferred into cultured cells in vitro, or transferred in vivo in the cells of the intended host. Techniques suitable for the transfer of nucleic acid into mammalian cells in vitro include the use of liposomes, electroporation, microinjection, cell fusion, DEAE-dextran, the calcium-phosphate precipitation method, etc. A commonly used vector for ex vivo delivery of the gene is a retrovirus.

The subject methods may generally be performed on biological samples from living subjects. A particularly advantageous feature of the invention is that the methods can simultaneously detect, in one reaction, several known oncogenic strains of HPV. In particular embodiments, the antibody composition of the invention may be employed in immunohistological examination of a sample.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Generation of Antibodies Specific for N-Terminal E6 Protein

In this example, two mAbs specific for the N-terminal E6 protein, 737BLT and 738BLT mAbs, were generated.

Mice

Female SJL mice (Taconic Hudson, N.Y.) or Balb/c mice (Charles River Laboratories Raleigh, N.C.) between 6 to 8 weeks old were obtained for use in antibody development. Mice were housed and immunized according to an approved Institutional Animal Care and Use Committee protocol.

Oncopeptide Construction

The sperm whale myoglobin amino acid sequence from 106-118 (FISEAIIHVLHSR) was used as a foreign T cell epitope. The B cell epitopes RRETQL or RRETQV were derived from the C-terminus of human HPV 16 E6 and HPV 18 E6 respectively. Single onco-peptides were synthesized by New England Peptide (Gardner, Mass.) with the generic format: H₂N-FISEAIIHVLHSR RRETQL-OH or H₂N-FISEAIIHVLHSR RRETQV-OH and provided as a lyophilized product. HPLC purity was >85%. Peptides were hydrated in DEPC water (Invitrogen, Carlsbad, Calif.) at 2.5 mg/ml and stored at 4° C.

Consensus Peptide Construction

The B cell consensus epitopes FQDPAERPRKLHDLCTEL (737BLT) and FQDPAERPYKLPDLCTEL (738BLT) were derived from the N-terminus of oncogenic human HPV E6. Chemical conjugation of peptides with the carrier proteins keyhole limpet hemocyanin (KLH) or ovalbumin (OVA) was performed by Peptron (Santa Clara, Calif.) and provided as a lyophilized product. Carrier/peptides were hydrated in DEPC water (Invitrogen, Carlsbad, Calif.) at 1 mg/ml and stored at 4° C.

Immunization of Mice with Oncopeptides

Two different immunizations were performed with either T cell epitope-RRETQL or T cell epitope-RRETQV using a rapid intrasplenic immunization (IS). Balb/c mice at 12 weeks of age were used for rapid IS immunization. Mice were anesthetized with 1.2 mg Ketamine-HCL (Bioniche Animal Health, Athens, Ga.) and 0.39 mg xylazine (Lloyd Laboratories, Shenandoah, Iowa) injected intraperitoneally prior to immunization. Mice were shaved with clippers to visualize the spleen under the skin on day 0. Thirty minutes after initial anesthesia mice were immobilized by 2% isoflurane gas (Butler Animal Health Supply, Dublin, Ohio) in an EZ Anesthesia vaporizer (Palmer, Pa.) according to product instructions. One-hundred micrograms of onco-peptide mixed with 5 μl Gerbu adjuvant was injected directly into the spleen in two sites in a total volume of 45 μl using a BD Ultrafine II insulin syringe (Franklin Lakes, N.J.) on days 0, 4, and 11. Fifty-micrograms of anti-CD40 agonist Mab clone 1C10 (R&D Systems, Minneapolis, Minn.) was injected in the subcutaneous tissue at the base of the tail in a 25 μl volume on day 10.²⁷ Mice were sacrificed on day 13 and spleens were collected for fusion.

Immunization of Mice with Consensus Peptides

Repetitive immunizations multiple site (RIMMS) protocols were performed in separate mice with each KLH-conjugated consensus peptide. SJL mice at 12 weeks of age were used for the RIMMS protocol. Prior to immunization, mice were anesthetized with 2% isoflurane (Butler Animal Health Supply, Dublin, Ohio) in an EZ Anesthesia vaporizer (Palmer, Pa.) according to manufacturer instructions. Fifteen micrograms of KLH-conjugated consensus peptide was emulsified in Freund's complete adjuvant (Sigma-Aldrich, St Louis, Mo.) and RIBI adjuvant (Ribi ImmunoChem Research, Inc., Hamilton, Mont.) for the initial day 0 immunization. Seven micrograms and 5 micrograms of consensus peptides were emulsified in RIBI adjuvant and injected on days 4 and 11 respectively. Multiple injections were made in the subcutaneous tissue with 50 μl/site to focus drainage into the popliteal, inguinal, axillary, and brachial lymph nodes. Mice were sacrificed on day 13 and the 8 bilateral lymph nodes were collected and pooled for fusion.

Cell Fusion

Lymphocytes were flushed from organs with 10 ml RPMI (Invitrogen, Carlsbad, Calif.), pelleted by centrifugation, and fused 1:1 with a stable Bcl-2 expressing P3×63.Ag8.653 myeloma cell line with 100 μl polyethylene glycol 1500 (Roche Diagnostics, Indianapolis, Ind.) per 10⁷ lymphocytes. Cells were resuspended in selection medium, (50% Ex-Cell™ 610-HSF [Lenexa, Kans.], 38% RPMI, 10% FBS [Hyclone, Logan, Utah], 1% penicillin-streptomycin-L glutamine, 1% non-essential amino acids [Invitrogen, Carlsbad, Calif.], 5.7 μM azaserine, 100 μM hypoxanthine [Sigma-Aldrich, St Louis, Mo.], 1 μg/L human IL-6) and seeded into 96-well tissue culture plates (Corning, Corning, N.Y.) at 5.3×10⁶ lymphocytes/plate for oncopeptides or 7×10⁶ lymphocytes/plate for consensus peptides. Non-hybridoma immunoglobulin, derived from non-fused B cells, was minimized by media replacement on days 7-9 post-fusion with feeding media (50% Ex-Cell™ 610-HFS, 33% RPMI, 1% penicillin-streptomycin-L glutamine, 1% non-essential amino acids, and 15% FBS). Resulting hybridomas were screened for target specificity by ELISA on day 12 using the immunogen and ovalbumin-conjugated-RRETQL or ovalbumin-conjugated-RRETQV for oncopeptide fusions and the respective immunogen and respective ovalbumin-conjugated consensus peptides. Positive cultures were expanded to 12-well tissue culture plates (Corning, Corning, N.Y.), grown to confluency in expansion media (50% Ex-Cell™ 610-HSF, 33% RPMI, 10% FBS, 1% penicillin-streptomycin-L glutamine, 1% non-essential amino acids, 1 μg/L, human IL-6, 5% Hybridoma Cloning Factor [Bioveris, Gaithersburg, Md.]), and frozen. Antibody containing supernatants were characterized by ELISA and Western blot.

Hybridoma Characterization: ELISA, Western Blot

High protein-binding polystyrene 96-well plates (Corning, Corning, N.Y.) were coated overnight with recombinant proteins at 1 μg/ml, onco-peptide immunogens and KLH-conjugated consensus peptide immunogens at 2 μg/ml, ovalbumin-conjugated-RRETQL, ovalbumin-conjugated-RRETQV, and ovalbumin-conjugated consensus peptides at 5 ug/ml in carbonate/bicarbonate coating buffer (pH 9.6). After washing in phosphate buffered saline with 0.05% Tween-20 (PBST) (Sigma-Aldrich, St Louis, Mo.), the wells were blocked with PBST containing 5% goat sera (Invitrogen, Carlsbad, Calif.) for a minimum of 1 hour at room temperature (RT). Undiluted culture supernatant was incubated on coated plates at 50 μl per well for 1 hour at RT. Mouse anti-His C-term antibody (Invitrogen, Carlsbad, Calif.) for recombinant Histidine-tagged proteins, mouse anti-Ovalbumin (Sigma-Aldrich, St Louis, Mo.) for ovalbumin-conjugates, mouse anti-KLH (Sigma-Aldrich, St Louis, Mo.) for KLH-conjugates, and expansion media were used as positive and negative controls. Plates were washed three times with PBST and probed for 1 hour at RT with HRP-labeled goat anti-mouse IgG (Southern Biotech, Birmingham, Ala.) which was diluted 1:1000 in PBST and 5% goat sera. Plates were washed five times with PBST and developed for 10 minutes at RT with TMB substrate (Millipore, Temecula, Calif.). Development was stopped by addition of an equal volume of 2.0N sulfuric acid (VWR, West Chester, Pa.). Absorbance was measured at 450 nm with an automated spectrophotometer.

For Western blots, recombinant proteins were reduced and denatured with NuPage® LDS sample buffer and sample reducing agent (Invitrogen, Carlsbad, Calif.), and then heated for 5 minutes at 95° C. Twenty micrograms of protein was separated by electrophoresis on 2D NuPage® 4-12% Bis-Tris gels (Invitrogen, Carlsbad, Calif.) with NuPage® MES SDS running buffer (Invitrogen, Carlsbad, Calif.) at 200 volts for 30 minutes. Proteins were transferred to nitrocellulose (Invitrogen, Carlsbad, Calif.) using NuPage® transfer buffer (Invitrogen, Carlsbad, Calif.) plus 10% methanol at 30 volts for 90 minutes. Membranes were blocked overnight at 4° C. with TBS-casein (Bio-Rad, Hercules, Calif.). Blots were assembled on a Miniblotter® 28 (Immunetics, Boston, Mass.). Non-diluted supernatant was loaded into individual slots and incubated for 1 hour at RT, and then washed extensively with PBST. Mouse anti-His C-term antibody (Invitrogen, Carlsbad, Calif.) or mouse Penta-His (Qiagen, Valencia, Calif.) and expansion media were used as positive and negative controls. Blots were then incubated with alkaline phosphatase (AP) labeled goat anti-mouse IgG (Southern Biotech, Birmingham, Ala.) at 1:1000 in TBS-casein for 1 hour at RT. Blots were extensively washed followed by development with Promega Western Blue® AP substrate (Promega, Madison, Wis.) at RT.

Purification of Monoclonal Antibodies

Hybridoma culture supernatants were diluted 1:1 with binding buffer (50 mM boric acid, 4 M sodium chloride, pH 9.0) and passed through a 5 ml HiTrap MabSelect SuRe™ protein A column (GE Healthcare, Piscataway, N.J.) using the AKTAxpress chromatography system. The column was washed with 10 column volumes of binding buffer followed by elution with 5 column volumes of 50 mM sodium citrate (pH 3.0), 50 mM sodium phosphate, 300 mM sodium chloride. Antibody eluates were stored in a sample loop and immediately buffer exchanged into 50 mM sodium phosphate (pH 7.4), 150 mM sodium chloride using a HiPrep 26/10 desalting column (GE Healthcare, Piscataway, N.J.). Antibody containing fractions were pooled and 0.2 μm filtered through a PES Supor membrane syringe filter (Pall Life Sciences, Ann Arbor, Mich.). Immunoglobulin levels were quantified using A₂₈₀ and purity was assessed using SDS-PAGE under reducing and non-reducing conditions.

Limit Dilution Cloning

Hybridomas were cloned by limit dilution into 2×96 well plates. Each well was microscopically observed to confirm the presence of a single cell. After incubation for 12 days, wells with growth originating from a single cell were screened by ELISA.

Expansion of Hybridomas

Selected monoclonal hybridomas were expanded to 500 ml in either Lampire Cell Culture Bags (Lampire, Pipersville, Pa.) or T-225 flasks (Corning, Corning N.Y.) and allowed to grow to exhaustion (<20% viable cells). Supernatant was decanted and cells were pelleted by centrifugation. Clarified supernatants were passed through a 0.2 μm PES filter (Nalge Nunc, Rochester, N.Y.) prior to purification.

### Example 2

### Specificity of the Subject Antibody for HPV E6 Oncopeptides

The antibodies generated by the method described in Example 1 were screened for specificity to high-risk HPV E6 oncoproteins by ELISA and Western blot. For the sandwich ELISA using hybridoma supernatant, capture mAb supernatant was diluted 1:7.5 in coating buffer and 50 μl/well was coated onto 96-well ELISA plates (Costar, Corning, N.Y.) overnight at 4° C. The plates were washed one time with PBST and wells were blocked with 300 μl 3% BSA/PBST for 1 hour at room temperature. The blocking solution was aspirated and 50 μl of antigen, diluted to 1 μg/ml in 1% BSA/PBST, was added to each well for 1 hour at room temperature. Detection mAb supernatant was diluted 1:7.5 in 1% BSA/PBST and incubated with horseradish peroxidase (HRP) labeled goat anti-mouse IgG-Fc antibody (Bethyl, Montgomery, Tex.) at a final concentration of 150 ng/ml at room temperature for 30 minutes. Plates were washed three times with PBST. An equal volume of 3 mg/ml mouse IgG (dialyzed to remove sodium azide, BioCheck, Foster City, Calif.) was added to the mAb-HRP detector complex prior to transferring 50 μl to each well for 1 hour at room temperature. Plates were washed three times with PBST followed by addition of 50 μl TMB substrate solution (Sigma, St. Louis, Mo.) per well. The reaction was allowed to develop for 10-30 minutes and was stopped with 2N sulfuric acid (VWR). Absorbance at 450 nm was read using a SpectraMax M2 plate reader (Molecular Devices, Sunnyvale, Calif.). Signal to noise ratios were determined by dividing the OD of wells with antigen by the OD of wells with buffer for each antibody pair. Positive antibody pairs were identified by having signal-to-noise ratios>2.0.

For sandwich ELISA using purified biotinylated mAb, the following changes were made to the protocol described above for using hybridoma supernatant. Purified capture mAbs were coated directly onto ELISA plates at 2 μg/ml. Purified biotinylated detection mAbs were diluted to 1 μg/ml in 1% BSA/PBST and 50 μl was added to each well followed by incubation for 1 hour at room temperature. After plates were washed three times with PBST, 50 μl of HRP-labeled streptavidin (Pierce, Rockford, Ill.), diluted 1:5000 in 1% BSA/PBST, was added to each well. The remainder of the assay was performed as previously described.

As shown in Tables 5 and 6, the antibodies generated via the subject method are specific for the N-terminal end of E6 proteins from high-risk HPV strains.

### Example 3

### Cross-Reactivity of the Subject Antibody to High-Risk HPV E6 Proteins

The high-affinity polyclonal antibodies were put through a round of limiting dilution as described in Example 1 to generate monoclonal hybridoma cell lines. Each monoclonal antibody was tested for cross reactivity in Western blots against purified E6 proteins from multiple HPV strains, for example, HPV16, 18, 33, 31, 35, 45, 52, 56, 58, 69, 11, and 6b as shown in Table 7. Western blotting was carried out as described in Example 1. Briefly, recombinant proteins were reduced and denatured with NuPage® LDS sample buffer and sample reducing agent (Invitrogen, Carlsbad, Calif.), and then heated for 5 minutes at 95° C. Twenty micrograms of protein was separated by electrophoresis on 2D NuPage® 4-12% Bis-Tris gels (Invitrogen, Carlsbad, Calif.) with NuPage® MES SDS running buffer (Invitrogen, Carlsbad, Calif.) at 200 volts for 30 minutes. Proteins were transferred to nitrocellulose (Invitrogen, Carlsbad, Calif.) using NuPage® transfer buffer (Invitrogen, Carlsbad, Calif.) plus 10% methanol at 30 volts for 90 minutes. Membranes were blocked overnight at 4° C. with TBS-casein (Bio-Rad, Hercules, Calif.). Blots were assembled on a Miniblotter® 28 (Immunetics, Boston, Mass.). Non-diluted supernatant was loaded into individual slots and incubated for 1 hour at RT, and then washed extensively with PBST. Mouse anti-His C-term antibody (Invitrogen, Carlsbad, Calif.) and expansion media were used as positive and negative controls. Blots were then incubated with alkaline phosphatase (AP) labeled goat anti-mouse IgG or IgM (Southern Biotech, Birmingham, Ala.) at 1:1000 in TBS-casein for 1 hour at RT. Blots were extensively washed followed by development with Promega Western Blue® AP substrate (Promega, Madison, Wis.) for 20 minutes at RT.

Table 7 shows “pan” binding and cross reactivity to E6 proteins from multiple high-risk HPV strains using the high-risk HPV consensus peptide mAbs of the present invention.

### Example 4

### Determining Complementary Epitopes of the Subject Antibodies Using Sandwich Assays

Sandwich assay was used to determine the complementary antibody pairs, i.e. capture and detection antibodies for detecting oncogenic E6 proteins from HPV 16 and HPV 18. The sandwich ELISA was carried out as described in details in Example 2. The antibody pairs that have complementary epitopes on E6 proteins are shown in Table 8.

### Example 5

### Detection of Antibody-E6 Binding Via Immunohistochemistry on Cell Lines Containing High-Risk HPV

The antibodies of the present invention are used in immunohistochemistry (IHC) to detect E6 proteins from ovarian tissues containing high-risk HPV strains on tissue slides. IHC is a well known technique in the art and the procedure is briefly disclosed herein. Formalin fixed paraffin-embedded ovarian tissues are cut into 4 μm sections and placed on superfrost+slides and baked at 60° C. for 20 minutes. Slides are stained using the Benchmark XT staining platform and Ventana reagents. The following staining parameters are used: antigen retrieval using Cell Conditioner 1 (standard), 11E4 antibody incubation at 37° C., (1 hour), DAB detection using I-VIEW detection kit, hematoxylin II, (counterstain), and bluing reagent. At the completion of staining run, slides are post-processed by washing in 1% Dawn dishwashing detergent solution followed by a 3 minute tap water rinse. Slides are dehydrated through a series of alcohols and xylene, mounted and coverslipped. The IHC results demonstrate that the antibodies specific for the N-terminus of E6 proteins bind and detect E6 proteins from high-risk HPV strains on HPV-containing tissues.

### Example 6

### Cross-Reactivity of Consensus Peptide Antibodies to HPV E6 Types in ELISA

In this example, five mAb clones specific for the N-terminal E6 protein, 1B2.27, 7F10.3, 4E9.7, 4E10.2, and 6H5.3, were used to detect E6 proteins from various HPV strains, HPV16, 18, 30, 31, 35, 45, 52, 53, 58, 59, 66, 68, 69, 6b, and 11 in an ELISA format. Recombinant HPV E6 proteins were purified and coated directly to microtiter plates or captured with a PDZ domain containing protein. Primary antibodies to the consensus peptides were diluted to 1 μg/ml and added to the wells. Binding was detected by addition of a secondary goat anti-mouse IgG:HRP followed by the substrate TMB. Signal to noise (S/N) ratios were calculated by dividing the OD₄₅₀ of test wells by the OD₄₅₀ from wells with no consensus peptide antibody. A more specific method of carrying out the sandwich ELISA is disclosed herein below:

Direct ELISA: High protein-binding polystyrene 96-well plates (Corning, Corning, N.Y.) were coated overnight with recombinant proteins at 1 μg/ml in carbonate/bicarbonate coating buffer (pH 9.6). After washing in phosphate buffered saline with 0.05% Tween-20 (PBST) (Sigma-Aldrich, St Louis, Mo.), the wells were blocked with PBST containing 3% BSA for a minimum of 1 hour at room temperature (RT). Purified monoclonal antibodies diluted to 1 μg/ml in PBST+1% BSA were incubated on coated plates at 50 μl per well for 1 hour at room temperature (RT). Plates were washed three times with PBST and probed for 1 hour at RT with HRP-labeled goat anti-mouse IgG (Southern Biotech, Birmingham, Ala.) which was diluted 1:1000 in PBST and 1% BSA. Plates were washed five times with PBST and developed for 10 minutes at RT with TMB substrate (Sigma-Aldrich, St Louis, Mo.). Development was stopped by addition of an equal volume of 2.0N sulfuric acid (VWR, West Chester, Pa.). Absorbance was measured at 450 nm with an automated spectrophotometer.

PDZ capture assay: High protein-binding polystyrene 96-well plates (Corning, Corning, N.Y.) were coated overnight with goat anti-GST diluted 1:1000 in carbonate/bicarbonate coating buffer (pH 9.6). After washing in phosphate buffered saline with 0.05% Tween-20 (PBST) (Sigma-Aldrich, St Louis, Mo.), the wells were blocked with PBST containing 3% BSA for a minimum of 1 hour at room temperature (RT). GST-PDZ MAGI diluted to 1 ug/ml in 1% BSA/PBST was added to the plates at 50 μl/well for one hour at RT. Plates were washed three times with PBST followed by addition of recombinant E6 protein diluted to 1 ug/ml in 1% BSA/PBST at 50 ul/well for one hour at RT. Plates were washed three times with PBST followed by addition purified monoclonal antibodies diluted to 1 μg/ml in 1% BSA/PBST at 50 μl/well for one hour at room temperature (RT). Plates were washed three times with PBST and probed for 1 hour at RT with HRP-labeled goat anti-mouse IgG (Southern Biotech, Birmingham, Ala.) which was diluted 1:1000 in 1% BSA/PBST. Plates were washed three times with PBST and developed for 10 to 30 minutes at RT with TMB substrate (Sigma-Aldrich, St Louis, Mo.). Development was stopped by addition of an equal volume of 2.0N sulfuric acid (VWR, West Chester, Pa.). Absorbance was measured at 450 nm with an automated spectrophotometer.

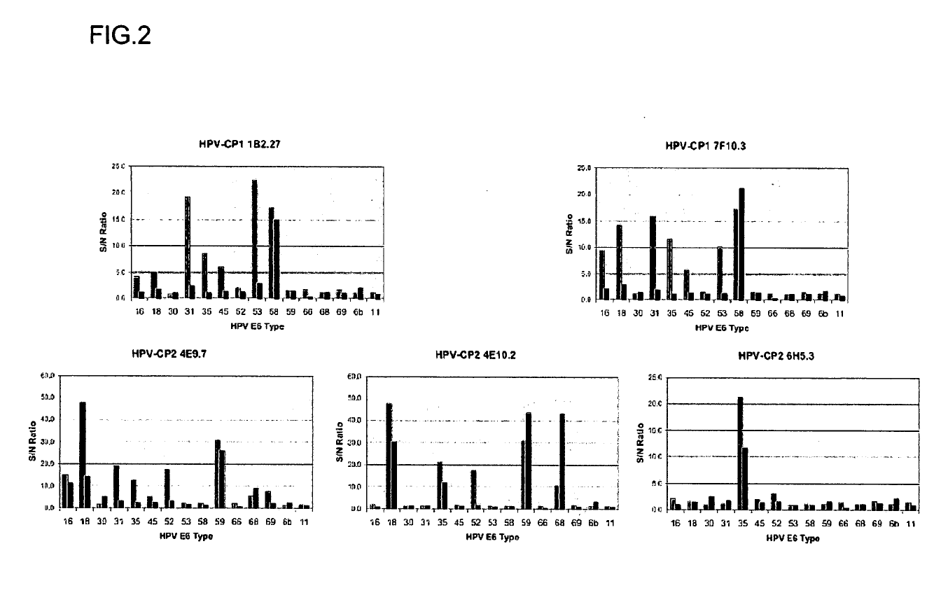

As shown in FIG. 2, cross-reactivity profiles varied greatly between the five antibodies. Clone 6H5.3 demonstrated monospecific binding, while clone 4E9.7 had the ability to bind 8 HPV E6 types in the direct ELISA format.

### Example 7

### Cross-Reactivity of Consensus Peptide Antibodies to HPV E6 Types in Western Blot

In this example, five mAb clones specific for the N-terminal E6 protein, 1B2.27, 7F10.3, 4E9.7, 4E10.2, and 6H5.3, were used to probe E6 proteins from various HPV strains, HPV16, 18, 30, 31, 35, 45, 52, 53, 58, 59, 66, 68, 69, 6b, and 11, in Western blots. Briefly, recombinant HPV E6 proteins were resolved by SDS-PAGE. Western blots were probed with the consensus peptide antibodies of the present invention. Goat anti-mouse IgG:AP was used to detect binding of the antibodies to E6.

More specifically, recombinant proteins were reduced and denatured with NuPage® LDS sample buffer and sample reducing agent (Invitrogen, Carlsbad, Calif.), and then heated for 8 minutes at 100° C. One microgram of protein was separated by electrophoresis on NuPage® 4-12% Bis-Tris gels (Invitrogen, Carlsbad, Calif.) with NuPage® MES SDS running buffer (Invitrogen, Carlsbad, Calif.) at 200 volts for 30 minutes. Proteins were transferred to nitrocellulose (Invitrogen, Carlsbad, Calif.) using the iBlot system (Invitrogen, Carlsbad, Calif.). Membranes were blocked with TBS-casein (Bio-Rad, Hercules, Calif.). Primary consensus peptide antibodies were diluted to 1 μg/ml in TBS-casein and incubated on the membranes at RT for one hour. Goat anti-mouse Penta-His antibody (Qiagen) was diluted 1:1000 in TBS-casein for use as a loading control. Blots were washed 3 times for 5 minutes each with TBST. Blots were then incubated with alkaline phosphatase (AP) labeled Fc-specific sheep anti-Mouse antibody (Jackson Immuno) diluted 1:5000 in TBS-casein for 1 hour at RT. Blots were washed 3 times for 5 minutes each with TBST followed by development with NBT/BCIP substrate (Promega, Madison, Wis.) for 10 to 20 minutes at RT.

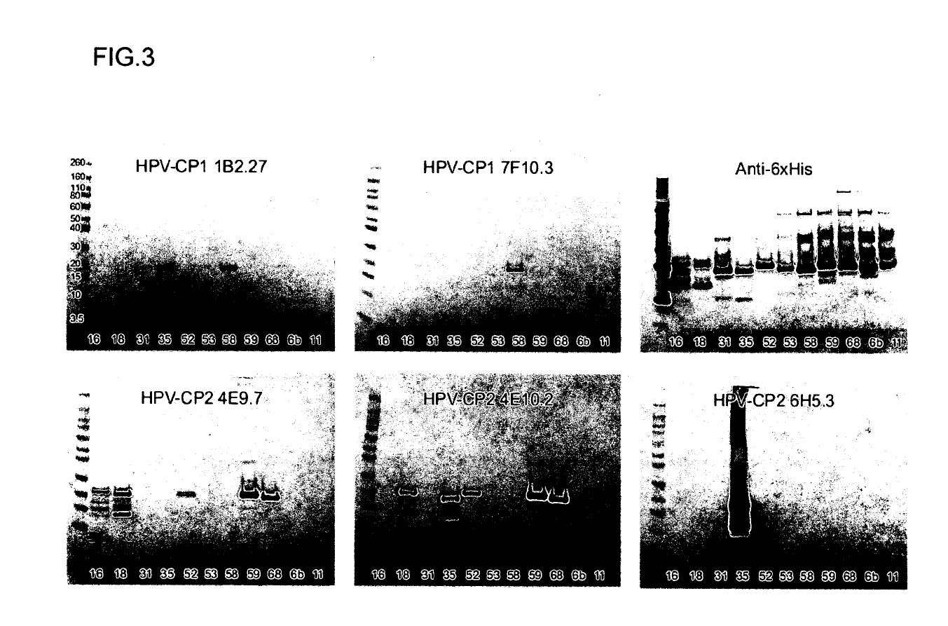

As shown in FIG. 3, the antibody cross-reactivity profiles ranged from being specific to a single HPV type to being specific for 5 or more HPV types. However, cross-reactivity of the antibodies of the present invention to low risk HPV types 6b and 11 was not observed, suggesting that the antibodies of the present invention were specific to the oncogenic E6 proteins from the high-risk HPV strains.

### Example 8

### Immunoprecipitation of Recombinant HPV-16 E6 by the Consensus Peptide Antibodies

In this example, five mAb clones specific for the N-terminal E6 protein, 1B2.27, 7F10.3, 4E9.7, 4E10.2, and 6H5.3, were used to immunoprecipitate E6 protein from HPV16. Briefly, the antibodies were linked to protein-G Dynabeads and incubated with recombinant maltose binding protein (MBP) tagged HPV-16 E6. After washing, the immune complexes were separated by SDS-PAGE followed by Western blotting with an HPV-16 E6 specific mouse antibody. An alkaline phosphatase conjugated anti-mouse light chain specific antibody was used to detect the immunoprecipitated HPV16 E6:MBP.

More specifically, protein-G Dynabeads (Invitrogen, Carlsbad, Calif.) were resuspended by vortexing for 20 seconds. Beads were transferred to a microfuge tube and then washed by magnetic separation/resuspension three times with 200 μl of RIPA buffer [50 mM Tris-HCl, 150 mM NaCl, 0.3% w/v deoxycholic acid, 1% v/v Triton X-100, 1 mM EDTA, pH 7.4]. Each immunoprecipitation (IP) reaction was set up with 50 μl of beads. Five μg of consensus peptide antibody was prepared in 200 μl of RIPA buffer and added to the beads for 30 minutes at RT with end-over-end rotation. Beads were washed 3 times with 200 μl of RIPA. Maltose binding protein (MBP) tagged HPV-16 E6 was diluted to 1 μg in 200 μl of RIPA and was then added to the beads. After a 30 minute incubation with the beads, unbound HPV-16 E6:MBP was removed by washing the beads 3 times with 200 μl of RIPA. After the third wash, the beads were transferred to a fresh microfuge tube and were then resuspended in 15 μl of NuPAGE sample buffer plus DTT (Invitrogen, Carlsbad, Calif.). Samples were heated at 100° C. for 8 minutes. One microgram of recombinant HPV-16 E6:MBP was added as a control for molecular weight and primary antibody binding. Proteins were separated by electrophoresis on NuPage® 4-12% Bis-Tris gels (Invitrogen, Carlsbad, Calif.) with NuPage® MES SDS running buffer (Invitrogen, Carlsbad, Calif.) at 200 volts for 30 minutes. Proteins were transferred to nitrocellulose (Invitrogen, Carlsbad, Calif.) using the iBlot system (Invitrogen, Carlsbad, Calif.). Membranes were blocked with TBS-casein (Bio-Rad, Hercules, Calif.). Primary mouse anti-HPV-16 E6 clone 2C4.12 (developed internally) was diluted to 1 μg/ml in TBS-casein and incubated on the membranes at RT for one hour. Blots were washed 3 times for 5 minutes each with TBST. Blots were then incubated with alkaline phosphatase (AP) labeled light chain specific goat anti-Mouse antibody (Jackson Immuno) diluted 1:2500 in TBS-casein for 1 hour at RT. Blots were washed 3 times for 5 minutes each with TBST followed by development with NBT/BCIP substrate (Promega, Madison, Wis.) for 10 to 20 minutes at RT.

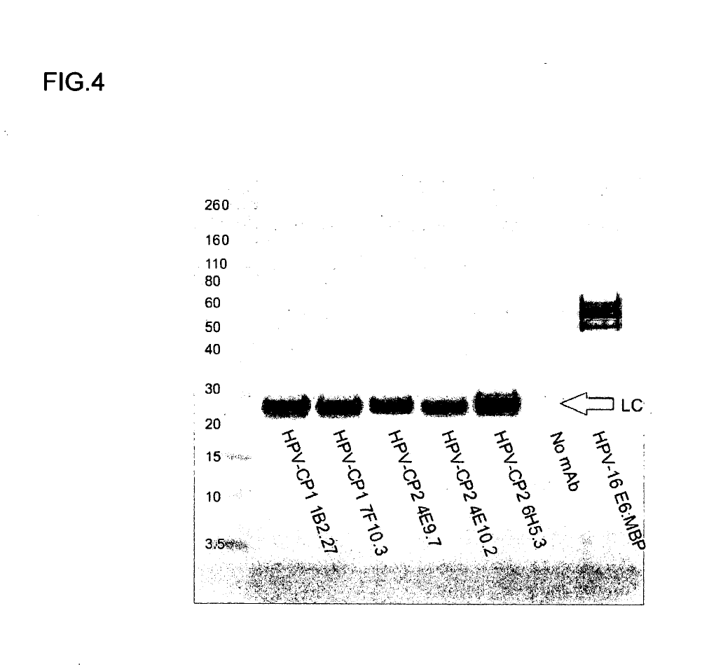

As shown in FIG. 4, mAb clone 4E9.7 was able to immunoprecipitate detectable levels of HPV16 E6:MBP.

### Example 9

### Detection of HPV-16 E6 from SiHa Cell Lysates by Sandwich ELISA Using a Consensus Peptide Capture Antibody

Purified consensus peptide antibody clone 4E9.7 was coated directly onto ELISA plates (Costar, Corning, N.Y.) at 16 μg/ml at 50 μl/well overnight at 4° C. The plates were washed one time with PBST and wells were blocked with 300 μl 3% BSA/PBST for 1 hour at room temperature. HPV-16 positive SiHa cells and HPV negative C33A− cells were lysed in RIPA buffer at 20 million cells per ml. In a separate mixing plate, 2-fold serial dilutions of the lysates were made in RIPA buffer. The blocking solution was aspirated and 100 μl of the lysate dilution series was added to the appropriate wells and allowed to incubate 1 hour at room temperature. Plates were washed three times with PBST. Biotinylated mouse anti-HPV-16 E6 detector antibody clone 6D3.5 (developed internally) was diluted to 0.25 μg/ml in 1% BSA/PBST and 50 μl was added to each well for one hour at RT. Plates were washed three times with PBST followed by addition of streptavidin-labeled horseradish peroxidase (Pierce, Rockford, Ill.) diluted 1:5000 in 1% BSA/PBST at 50 μl per well. Plates were washed three times with PBST followed by addition of 50 μl TMB substrate solution (Sigma, St. Louis, Mo.) per well. The reaction was allowed to develop for 30-60 minutes and was stopped with 50 μl per well of 2N sulfuric acid (VWR). Absorbance at 450 nm was read using a SpectraMax M2 plate reader (Molecular Devices, Sunnyvale, Calif.). Assay points were run in triplicate.

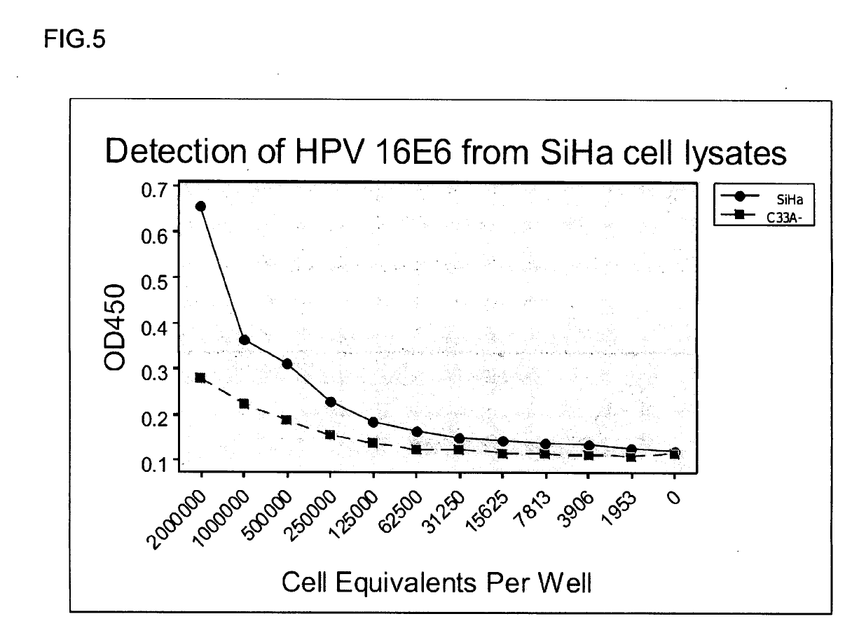

As shown in FIG. 5, HPV-16 E6 was detected from less than 5,000 SiHa cell equivalents using the mAb clone 4E9.7 of the present invention.

### Materials and Methods for Examples 10-12

Immunization Protocol:

Two different immunization strategies are used: repetitive immunizations multiple sites (RIMMS) and rapid intrasplenic immunization (IS). SJL mice at 12 weeks of age are used for the RIMMS protocol. Prior to immunization, mice are anesthetized with 2% isoflurane (Butler Animal Health Supply, Dublin, Ohio) in an EZ Anesthesia vaporizer (Palmer, Pa.) according to manufacturer instructions. Fifty-micrograms of recombinant ETO B.003 is emulsified in either Freund's complete adjuvant (Sigma-Aldrich, St Louis, Mo.) and 5 μl Gerbu adjuvant MM (Gerbu Biotechnik GMbH, Gaiberg, Germany) for the initial day 0 immunization, or Titermax adjuvant (Norcross, Ga.) and 5 μl Gerbu adjuvant MM for day 4 and 11 immunizations. Multiple injections are made in the subcutaneous tissue with 50 μl/site to focus drainage into the popliteal, inguinal, axillary, and brachial lymph nodes. Mice are sacrificed on day 13 and the 8 bilateral lymph nodes along with 2 lumbar lymph nodes are collected and pooled for fusion.

A Balb/c mouse at 12 weeks of age is used for rapid IS immunization. The mouse is anesthetized with 1.2 mg Ketamine-HCL (Bioniche Animal Health, Athens, Ga.) and 0.39 mg xylazine (Lloyd Laboratories, Shenandoah, Iowa) injected intraperitoneally prior to immunization. The mouse is shaved with clippers to visualize the spleen under the skin on day 0. Thirty minutes after initial anesthesia the mouse is placed in 2% isoflurane gas until immobile. One-hundred micrograms of ETO chimeric peptide mixed with 5 μl Gerbu adjuvant is injected directly into the spleen in two sites in a total volume of 150 μl using a BD Ultrafine II insulin syringe (Franklin Lakes, N.J.) on days 0, 4, and 11. Fifty-micrograms of anti-CD40 agonist Mab clone 1C10 (R&D Systems, Minneapolis, Minn.) is injected in the subcutaneous tissue at the base of the tail in a 25 μl volume on day 10. The mouse is sacrificed on day 13 and the spleen is collected for fusion.

In this example, mice are immunized on day zero with 20 μg of the following peptide F-I-S-E-A-I-I-H-V-L-H-S-R-C-R-Q-C-W-R-R-R-R-R-E-T-Q-L (in Table 2A) and 20 μg of polyl/polyC polymer or complete Freund's adjuvant. Animals are boosted with 20 μg of E6 protein and polyl/polyC or incomplete Freund's adjuvant. Test bleeds are performed 3 days after the last boost and screened by ELISA against the corresponding E6 peptide F-I-S-E-A-I-I-H-V-L-H-S-R-C-R-Q-C-W-R-R-R-R-R-E-T-Q-L or F-I-S-E-A-I-I-H-V-L-H-S-R-C-R-Q-C-W-R-R-R. Immunoreactive mice have a final boost three days prior to fusion.

A variety of immunization protocols including varying antigen doses (100 μg-10 μg), adjuvants (CFA/IFA, poly(I)-poly(C), CpG+Alum) and routes (subcutaneous, intraperitoneal) are tested. Immunization projects are set up with 5-15 mice each. Sera of immunized mice are tested in ELISA against the recombinant E6 peptide. Mice showing sufficiently high titers (OD above 1 at 1:1000 dilution) against E6 peptide in their sera are selected for fusions.

ELISA Screening of Serum Antibody Titer and B Cell Hybridoma Supernatants:

ELISA plates are coated with appropriate fusion protein, washed, and blocked with PBS containing 2% BSA (Sigma). Then the test sample (immune sera or hybridoma supernatant) is added, along with a pre-immune or irrelevant supernatant negative control. After incubation the plate is washed and anti-mouse IgG-HRP conjugate (Jackson Laboratories) in PBS/2% BSA is added. After thorough washing, TIME substrate is added for 30 minutes, followed by termination of the reaction with 0.18 M H₂SO₄. The plate is then read at 450 nm using a Molecular Devices' THERMO Max microplate reader.

Cell Fusion:

On the day of fusion, the animals are sacrificed and the lymphocytes are flushed from organs with 10 ml RPMI (Invitrogen, Carlsbad, Calif.), pelleted by centrifugation, and fused 1:1 with a stable Bcl-2 expressing P3×63.Ag8.653 myeloma cell line with 100 μl polyethylene glycol 1500 (Roche Diagnostics, Indianapolis, Ind.) per 10⁷ lymphocytes. Cells are resuspended in selection medium, (50% Ex-Cell™ 610-HSF [Lenexa, Kans.], 38% RPMI, 10% FBS [Hyclone, Logan, Utah], 1% penicillin-streptomycin-L glutamine, 1% non-essential amino acids [Invitrogen, Carlsbad, Calif.], 5.7 μM azaserine, 100 μM hypoxanthine [Sigma-Aldrich, St Louis, Mo.], 1 μg/L human IL-6) and seeded into 96-well tissue culture plates (Corning, Corning, N.Y.) at 10⁷ lymphocytes/plate for RIMMS or 2×10⁶ lymphocytes/plate for IS fusions. Non-hybridoma immunoglobulin, derived from non-fused B cells, is minimized by media replacement on days 7-9 post-fusion with feeding media (50% Ex-Cell™ 610-HFS, 33% RPMI, 1% penicillin-streptomycin-L glutamine, 1% non-essential amino acids, and 15% FBS). Resulting hybridomas are screened for target specificity by ELISA on day 12 using the immunogen and an irrelevant 6× histidine tagged protein control. Positive cultures are expanded to 12-well tissue culture plates (Corning, Corning, N.Y.), grown to confluency in expansion media (50% Ex-Cell™ 610-HSF, 33% RPMI, 10% FBS, 1% penicillin-streptomycin-L glutamine, 1% non-essential amino acids, 1 μg/L human IL-6, 5% Hybridoma Cloning Factor [Bioveris, Gaithersburg, Md.]), and frozen. Antibody containing supernatants are characterized by ELISA and Western blot.

### Purification of Monoclonal Antibodies

Hybridoma culture supernatants are diluted 1:1 with binding buffer (50 mM boric acid, 4 M sodium chloride, pH 9.0) and passed through a 5 ml HiTrap MabSelect SuRe™ protein A column (GE Healthcare, Piscataway, N.J.) using the AKTAxpress chromatography system. The column is washed with 10 column volumes of binding buffer followed by elution with 5 column volumes of 50 mM sodium citrate (pH 3.0), 50 mM sodium phosphate, 300 mM sodium chloride. Antibody eluates are stored in a sample loop and immediately buffer exchanged into 50 mM sodium phosphate (pH 7.4), 150 mM sodium chloride using a HiPrep 26/10 desalting column (GE Healthcare, Piscataway, N.J.). Antibody containing fractions are pooled and 0.2 μm filtered through a PES Supor membrane syringe filter (Pall Life Sciences, Ann Arbor, Mich.). Immunoglobulin levels are quantified using A₂₈₀ and purity is assessed using SDS-PAGE under reducing and non-reducing conditions.

### Limit Dilution Cloning

Hybridomas are cloned by limit dilution into 2×96 well plates. Each well is microscopically observed to confirm the presence of a single cell. After incubation for 12 days, wells with growth originating from a single cell are screened by ELISA.

### Expansion of Hybridomas

Selected monoclonal hybridomas are expanded to 500 ml in Lampire Cell Culture Bags (Lampire, Pipersville, Pa.) and allowed to grow to exhaustion (<20% viable cells). Supernatant is decanted and cells are pelleted by centrifugation. Clarified supernatants are passed through a 0.2 μm PES filter (Nalge Nunc, Rochester, N.Y.) prior to purification.

### Example 10

### HPV-E6 Recombinant Protein Expression and Purification

Polynucleotides encoding the PDZ domain binding motif in the C-terminus of oncogenic E6 proteins of high-risk HPV strains, for example, HPV16 and HPV18, are chemically synthesized (DNA 2.0, Menlo Park, Calif.) or cloned via RT-PCR from cervical cancer cell lines. The peptides C-R-Q-C-W-R-R-R-R-R-E-T-Q-L and C-R-Q-C-W-R-R-R-R-R-E-T-Q-V are fused with a T cell epitope F-I-S-E-A-I-I-H-V-L-H-S-R to generate a recombinant fusion peptide. The recombinant E6 fusion proteins are used as the immunogens. Generation of recombinant proteins is well established in the art and can be achieved via standard protocols. Proteins are expressed in DH5α E. coli using IPTG driven induction. A 2 h induction at 37° C. yields the immunizing peptides at ˜1 mg/L, whereas induction overnight at 20° C. and purification including rebinding of protein to the gel matrix results in final yield of 2-10 mg/L. Purity of the immunizing peptides is estimated to be >90% based on PAGE analysis.

### Example 11

### Generation of Hybridomas Secreting Antibodies Specific for C-Terminus of E6 Proteins

Hybridoma supernatants are tested via direct antigen ELISA against a screening peptide F-I-S-E-A-I-I-H-V-L-H-S-R-C-R-Q-C-W-R-R-R-R-R-E-T-Q-V. A non-E6 peptide is used as a negative control. Supernatants that show reactivity for the immunogen but not for the non-E6 peptide are selected for further analysis. Selected supernatants are tested further by slot western blot for reactivity against a different screening peptide F-I-S-E-A-I-I-H-V-L-H-S-R-C-R-Q-C-W-R-R-R or R-R-E-T-Q-V to reconfirm the presence of anti-E6 mAb. At this stage, hybridomas are cloned by limiting dilution to isolate hybridoma clones secreting anti-E6 mAb.

To further characterize the reactivity of the hybridomas, selected supernatants are tested in an ELISA against the recombinant E6 proteins, as well as GST-INADL (PDZ) and GST-MAGI1-PDZ1 that serve as negative controls. GST-INADL represents a class of proteins that, when purified in prokaryotic expression systems, tend to be associated with a bacterial contaminant that is also present in the MBP-/GST-E6 protein preparations used for immunizations. This control ensures that reactivity found in supernatants reflected a mAb binding to HPV-E6, and not against the associated contaminants.

### Example 12

### Specificity of the Subject Antibody for HPV E6 Oncopeptides

The specific binding of the subject antibodies, in this example, 6D9.3 and 1A9.1 mAbs, to oncogenic E6 proteins from HPV16 and HPV18 is measured by ELISA and Western blot. Binding of the subject antibodies to E6 protein from a non-oncogenic HPV strain, for example, HPV11 is used as the negative control. The E6 proteins used in this example are His tagged and the anti-His mAb is used as the positive control.

For ELISA, high protein-binding polystyrene 96-well plates (Corning, Corning, N.Y.) are coated overnight with recombinant proteins at 1 μg/ml or chimeric peptide at 2 μg/ml in carbonate/bicarbonate coating buffer (pH 9.6). After washing in phosphate buffered saline with 0.05% Tween-20 (PBST) (Sigma-Aldrich, St Louis, Mo.), the wells are blocked with PBST containing 5% goat sera (Invitrogen, Carlsbad, Calif.) for a minimum of 1 hour at room temperature (RT). Undiluted culture supernatant is incubated on coated plates at 50 μl per well for 1 hour at RT. Mouse anti-His C-term antibody (Invitrogen, Carlsbad, Calif.) and expansion media are used as positive and negative controls. Plates are washed three times with PBST and probed for 1 hour at RT with HRP-labeled goat anti-mouse IgG (Southern Biotech, Birmingham, Ala.) which is diluted 1:1000 in PBST and 5% goat sera. The original screen of chimeric peptide derived cultures is additionally probed with HRP-labeled goat anti-mouse IgM (Southern Biotech, Birmingham, Ala.). Subsequent screens of chimeric peptide antibodies are probed with isotype specific HRP-conjugates. Plates are washed five times with PBST and developed for 10 minutes at RT with TMB substrate (Millipore, Temecula, Calif.). Development is stopped by addition of an equal volume of 2.0N sulfuric acid (VWR, West Chester, Pa.). Absorbance is measured at 450 nm with an automated spectrophotometer.

For Western blots, recombinant proteins are reduced and denatured with NuPage® LDS sample buffer and sample reducing agent (Invitrogen, Carlsbad, Calif.), and then heated for 5 minutes at 95° C. Twenty micrograms of protein is separated by electrophoresis on 2D NuPage® 4-12% Bis-Tris gels (Invitrogen, Carlsbad, Calif.) with NuPage® MES SDS running buffer (Invitrogen, Carlsbad, Calif.) at 200 volts for 30 minutes. Proteins are transferred to nitrocellulose (Invitrogen, Carlsbad, Calif.) using NuPage® transfer buffer (Invitrogen, Carlsbad, Calif.) plus 10% methanol at 30 volts for 90 minutes. Membranes are blocked overnight at 4° C. with TBS-casein (Bio-Rad, Hercules, Calif.). Blots are assembled on a Miniblotter® 28 (Immunetics, Boston, Mass.). Non-diluted supernatant is loaded into individual slots and incubated for 1 hour at RT, and then washed extensively with PBST. Mouse anti-His C-term antibody (Invitrogen, Carlsbad, Calif.) and expansion media are used as positive and negative controls. Blots are then incubated with alkaline phosphatase (AP) labeled goat anti-mouse IgG or IgM (Southern Biotech, Birmingham, Ala.) at 1:1000 in TBS-casein for 1 hour at RT. Blots are extensively washed followed by development with Promega Western Blue® AP substrate (Promega, Madison, Wis.) for 20 minutes at RT.

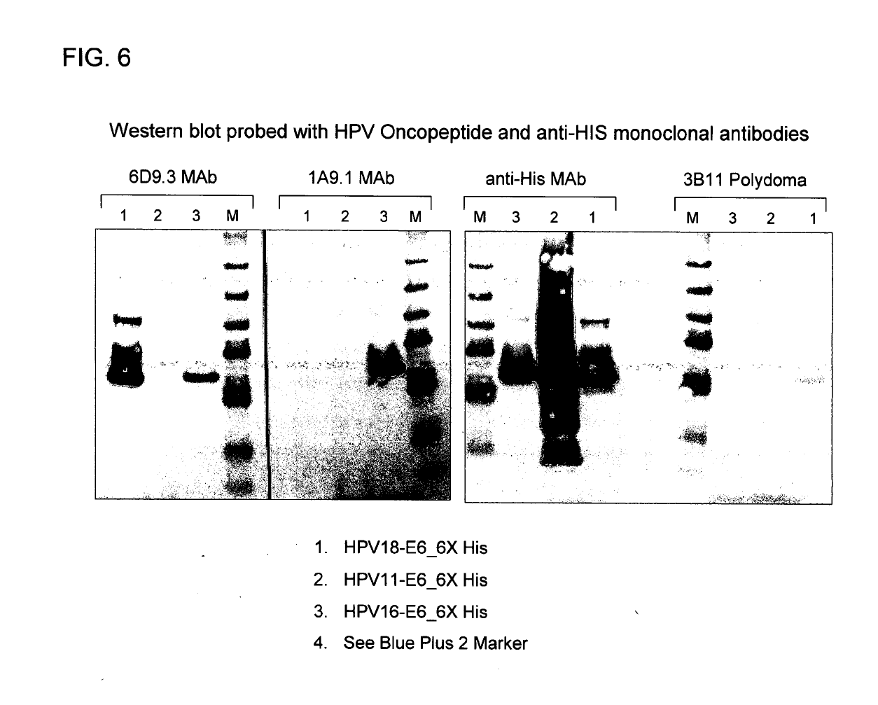

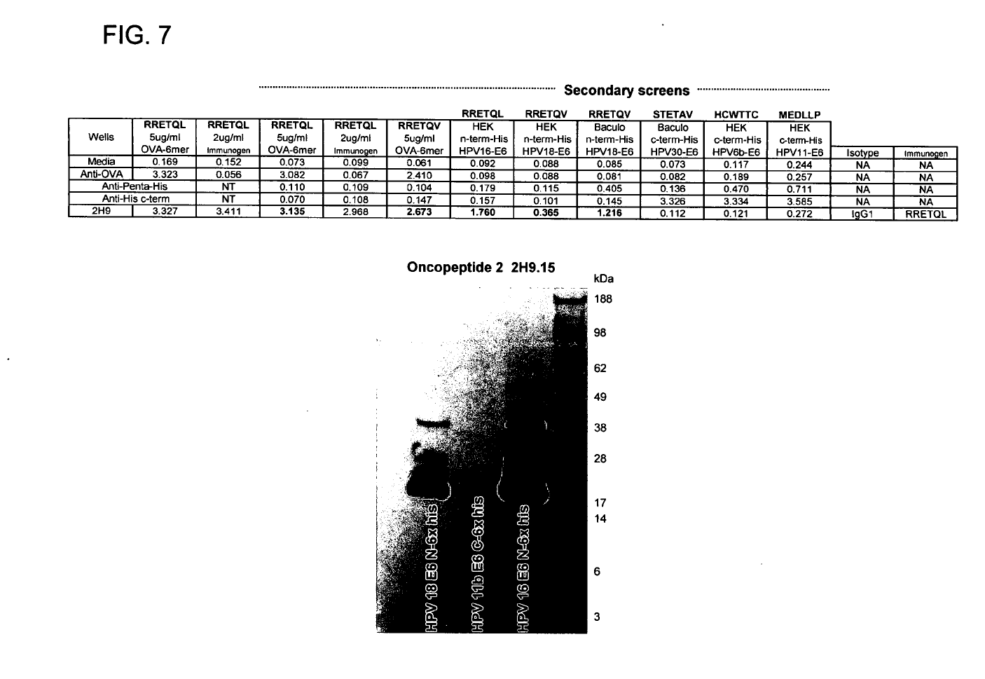

The Western results show that 6D9.3 mAb binds to E6 proteins from HPV18 and to a lesser extent to E6 proteins from HPV16. 1A9.1 mAb binds to E6 proteins from HPV16. Both mAbs are specific for oncogenic E6 proteins from high-risk HPV strains and therefore do not bind to E6 proteins from the low-risk HPV11 strain (FIG. 6). 2H9.15 is another mAb that binds to the C-terminus of E6 proteins from both HPV16 and HPV18, but not HPV11, as shown in FIG. 7. The results suggest that the subject antibodies specifically bind to more than one oncogenic E6 protein from high-risk HPV strains.

The ELISA results are shown in Table 10. The results show that 6D9.3 mAb binds to E6 proteins from HPV18 and to a lesser extent to E6 proteins from HPV16. 1A9.1 mAb binds to E6 proteins from HPV16. Both mAbs are specific for oncogenic E6 proteins from high-risk HPV strains and therefore do not bind to E6 proteins from the low-risk HPV11 strain. 2H9.15 is another mAb that binds to the C-terminus of E6 proteins from both HPV16 and HPV18, but not HPV11. The results suggest that the subject antibodies can specifically bind to more than one oncogenic E6 protein from high-risk HPV strains.

TABLE 10

| | ...RRETQL | ...RRETQL | ...RRETQV | ...RRETQL | ...RRETQL | ...RRETQV | ...RRETQL |
|---|---|---|---|---|---|---|---|
| See note a: | OVA-6mer | Carrier-hapten | OVA-6mer | OVA-6mer | Carrier-hapten | OVA-6mer | HPV16-E6^{b} |
| Media only | − | − | − | − | − | − | − |
| Anti-OVA | + | − | + | + | − | + | − |
| Anti-His | − | NT^{g} | NT^{g} | − | − | − | + |
| 2H9 | + | + | NT^{g} | + | + | + | + |
| 1A9 | + | + | NT^{g} | + | + | +/− | + |
| 6D9 | +/− | NT^{g} | + | − | NT^{g} | + | NT^{g} |

| | | Secondary screens | | | | Carrie [symbol] |
|---|---|---|---|---|---|---|
| | ...RRETQV | ...RRETQV | ...STETAV | ...HCWTTC | ...MEDLLP | hapte [symbol] |
| See note a: | HPV18-E6^{b} | HPV18-E6^{c} | HPV30-E6^{d} | HPV6b-E6 [symbol] | HPV11-E6 [symbol] | immuno [symbol] |
| Media only | − | − | − | − | − | NA [symbol] |
| Anti-OVA | − | − | − | − | − | NA [symbol] |
| Anti-His | + | + | + | + | + | NA [symbol] |
| 2H9 | + | + | − | − | − | RRET [symbol] |
| 1A9 | − | − | − | − | − | RRET [symbol] |
| 6D9 | NT^{g} | NT^{g} | NT^{g} | NT^{g} | NT^{g} | RRET [symbol] |

Table 10
^{a}Amino acid hapten sequence plus the amino acid sequence at most distal C-terminal end of HPV types involved with PDZ binding.
^{b}HEK expressed rHPV E6 with N-terminal hexahistidine tag.
^{c}Baculovirus expressed recombinant HPV E6 with an N-terminal hexahistidine tag.
^{d}Baculovirus expressed recombinant HPV E6 with a C-terminal hexahistidine tag.
^{e}HEK-293 expressed recombinant HPV E6 with a C-terminal hexahistidine tag.
^{f}Not applicable.
^{g}Not tested. [symbol]
indicates data missing or illegible when filed

### Example 13

### Capture of Oncogenic E6 Protein by the Subject Antibody Using Sandwich ELISA

Two purified monoclonal antibodies (mAb), 6D9.3 and 1A9.1, are produced by the hybridoma cell lines which are derived from somatic fusion of mouse P3×63-Ag-653/Bcl-2 myeloma cells with murine B cells obtained from the immunized mice. 6D9.3 and 1A9.1 mAbs are specific against the PDZ domain binding motif in the C-terminal region of oncogenic E6 proteins. In this Example, 6D9.3 and 1A9.1 are tested as the capture antibodies in a sandwich ELISA using various biotinylated anti-E6 antibodies as the detector antibodies. The antigen used in this Example is peptide 913BLT HPV16 HEK from HPV16. The capture mAbs 6D9.3 and 1A9.1 are used with various detector anti-E6 antibodies in different combinations (Table 11).

The sandwich ELISA is carried out as described herein. For sandwich ELISA using the supernatant, the capture mAb supernatant is diluted 1:7.5 in coating buffer and 50 μl/well is coated onto 96-well ELISA plates (Costar, Corning, N.Y.) overnight at 4° C. The plates are washed one time with PBST and wells are blocked with 300 μl 3% BSA/PBST for 1 hour at room temperature. The blocking solution is aspirated and 50 μl of antigen, diluted to 1 μg/ml in 1% BSA/PBST, is added to each well for 1 hour at room temperature. Detection mAb supernatant is diluted 1:7.5 in 1% BSA/PBST and incubated with horseradish peroxidase (HRP) labeled goat anti-mouse IgG-Fc antibody (Bethyl, Montgomery, Tex.) at a final concentration of 150 ng/ml at room temperature for 30 minutes. Plates are washed three times with PBST. An equal volume of 3 mg/ml mouse IgG (dialyzed to remove sodium azide, BioCheck, Foster City, Calif.) is added to the mAb-HRP detector complex prior to transferring 50 μl to each well for 1 hour at room temperature. Plates are washed three times with PBST followed by addition of 50 μl TMB substrate solution (Sigma, St. Louis, Mo.) per well. The reaction is allowed to develop for 10-30 minutes and is stopped with 2N sulfuric acid (VWR). Absorbance at 450 nm is read using a SpectraMax M2 plate reader (Molecular Devices, Sunnyvale, Calif.). Signal to noise ratios are determined by dividing the OD of wells with antigen by the OD of wells with buffer for each antibody pair. Positive antibody pairs are identified by having signal-to-noise ratios>2.0.

To test the purified mAbs and purified biotinylated mAbs in a sandwich ELISA, the following changes are made to the protocol described above for supernatant. Purified capture mAbs are coated directly onto ELISA plates at 2 μg/ml. Purified biotinylated detection mAbs are diluted to 1 μg/ml in 1% BSA/PBST and 50 μl is added to each well followed by incubation for 1 hour at room temperature. After plates are washed three times with PBST, 50 μl of HRP-labeled streptavidin (Pierce, Rockford, Ill.), diluted 1:5000 in 1% BSA/PBST, is added to each well. The remainder of the assay is performed as previously described.

The results of the mAb-E6 binding assay including the average signal-to-noise ratio (S/N ratio) are summarized in Table 11. The results show that 6D9.3 and 1A9.1 mAbs can bind and capture E6 protein of HPV16.

TABLE 11

| | Biotinylated | | Avg S/N Ratio |
|---|---|---|---|
| Capture MAb | Detector MAb | Antigen | (N = 2) |
| Oncopeptide 1 | AV 4C6 | 913BLT HPV 16 | 71.3 |
| 6D9.3 | | HEK | |
| Oncopeptide 1 | AV 8G11 | 913BLT HPV 16 | 58.1 |
| 6D9.3 | | HEK | |
| Oncopeptide 1 | 743BLT | 913BLT HPV 16 | 51.9 |
| 6D9.3 | 6D3.5 | HEK | |
| Oncopeptide 2 | AV 8G11 | 913BLT HPV 16 | 48.5 |
| 1A9.1 | | HEK | |
| Oncopeptide 1 | 876BLT | 913BLT HPV 16 | 45.6 |
| 6D9.3 | 11E10.30 | HEK | |
| Oncopeptide 2 | AV 4C6 | 913BLT HPV 16 | 41.4 |
| 1A9.1 | | HEK | |
| Oncopeptide 1 | 743BLT | 913BLT HPV 16 | 31.7 |
| 6D9.3 | 8H11.23 | HEK | |
| Oncopeptide 2 | 876BLT | 913BLT HPV 16 | 31.4 |
| 1A9.1 | 11E10.30 | HEK | |
| Oncopeptide 1 | 743BLT | 913BLT HPV 16 | 30.1 |
| 6D9.3 | 6D8.30 | HEK | |
| Oncopeptide 2 | 743BLT | 913BLT HPV 16 | 29.3 |
| 1A9.1 | 8H11.23 | HEK | |

### Example 14

### Blocking of HPV E6 Binding to PDZ Domain Protein by Antibodies Specific for C-Terminus of E6

An inhibition assay was carried out to assess the ability of antibodies specific for the C-terminal end of the HPV E6 protein, i.e. oncopeptides 2H9.15 and 6D9.3, to compete with a PDZ domain containing protein, MAGI-1, for binding to HPV E6. Briefly, the assay was performed as follows: a 96-well plate was coated with goat anti-GST mAb diluted at 1:1000 in carbonate/bicarbonate buffer at −4° C. overnight. The plate was washed one time and blocked with 3% BSA/PBST for 1 hour at room temperature (RT). GST-PDZ MAGI protein diluted to 1 ug/ml in 1% BSA/PBST was added to the plate and incubated for 1 hour at RT. The antigen, i.e. HPV E6 protein was diluted to 2 ug/ml in the diluent (1% BSA/PBST) and combined with an equal volume of the oncopeptide, i.e. mAb 2H9.15 or 6D9.3 in the diluent or an equal volume of the diluent not containing the mAb 2H9.15 as a control. The mixture was incubated for 30 minutes at RT. The plate was washed 3 times and 50 μl of the E6/mAb mixture or the E6 control without the mAb was added to each well and incubated for 1 hour at RT. The plate was washed 3 times. A biotinylated mAb against E6 was diluted to 0.25 ug/ml in 1% BSA/PBST and added to the plate for 1 hr at RT. The plate was washed 3 times. Streptavidin-HRP was diluted 1:5000 in 1% BSA/PBST and added to the plate for 1 hr at RT. The plate was again washed 3 times and developed with TMB for 10-30 min at RT. The color development was stopped with 2N sulfuric acid and the plate was read at 450 nm.

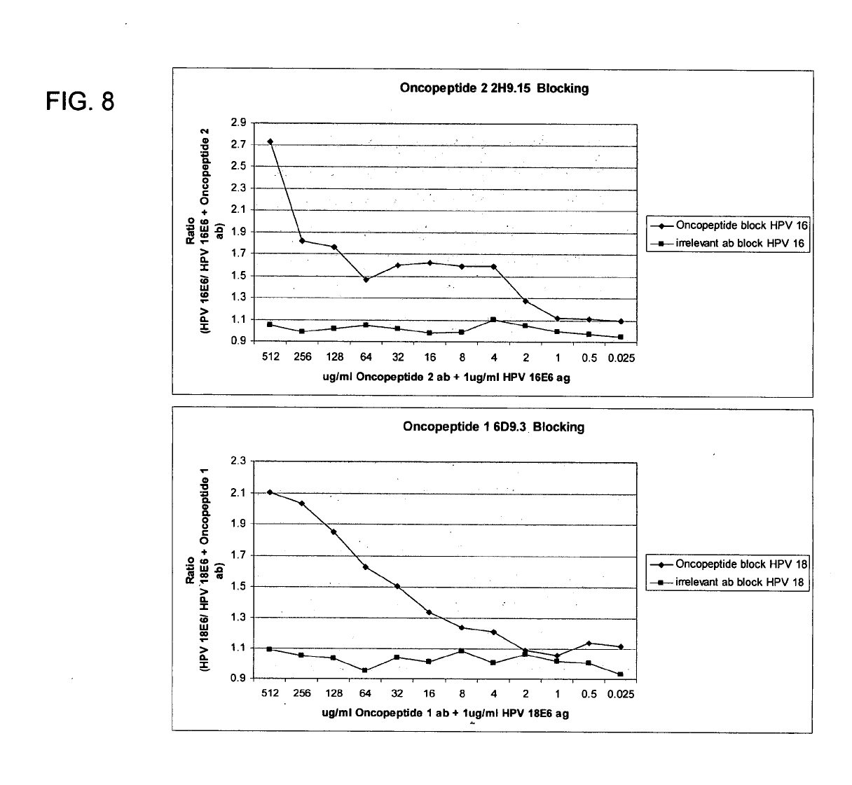

The results of the blocking experiment are shown in FIG. 8. Oncopeptides 2H9.15 and 6D9.3 blocked the binding of HPV E6 protein to the MAGI-1 PDZ binding domain, demonstrating that these antibodies specifically bind to the C-terminus of HPV E6 protein.

### Example 15

### Cross-Reactivity Patterns of Monoclonal Antibodies Specific for C-Terminus of Oncogenic E6 Proteins

The cross-reactivity patterns of anti-E6 mAb against E6 C-terminus other than the one used as immunogen are tested. For this test, a direct ELISA approach is used (recombinant E6 protein is coated on the plate).

Monoclonal antibodies against the E6 protein of the high-risk HPV types that cause cervical cancer (e.g., HPV 16, 18, 26, 30, 31, 34, 45, 51; 52, 53, 58, 59, 66, 68b, 69, 70, 73, 82) are produced. Some antibodies specifically bind to E6 proteins from at least two oncogenic strains of HPV. In some embodiments, the antibodies specific for the C-terminus of oncogenic E6 proteins bind to amino acid motifs that are conserved between the E6 proteins of different HPV strains, particularly HPV strains 16 and 18. In some embodiments, the antibodies specific for the C-terminus of oncogenic E6 proteins bind to amino acid motifs that are conserved between the E6 proteins of HPV strains 16 and 45.

TABLE 12

| | MAb | | | | | | | | | | | | | |
|---|---|---|---|---|---|---|---|---|---|---|---|---|---|---|
| Immunization Method | 16 | 18 | 6b | 11 | 30 | 31 | 35 | 45 | 52 | 58 | 59 | 66 | 68 | 69 |
| IS 6D9.3 | X | +/− | | | | | | | | | | +/− | | |
| IS 2H9.15 | X | | | | | X | X | X | +/− | | | | | |
| IS 1A9.1 | X | X | | | X | | X | X | | X | | | | |

As shown in Table 12, purified monoclonal antibodies were tested in ELISA for binding to recombinant HPV E6 proteins. Cross reactivity to the low-risk types HPV 6b and 11 was not detected.

### Example 16

### Detection of Antibody-E6 Binding Via Immunohistochemistry on Cell Lines Containing High-Risk HPV

The antibodies of the present invention are used in immunohistochemistry (IHC) to detect E6 proteins from ovarian tissues containing high-risk HPV strains on tissue slides. IHC is a well known technique in the art and the procedure is briefly disclosed herein. Formalin fixed paraffin-embedded ovarian tissues are cut into 4 μm sections and placed on superfrost+slides and baked at 60° C. for 20 minutes. Slides are stained using the Benchmark XT staining platform and Ventana reagents. The following staining parameters are used: antigen retrieval using Cell Conditioner 1 (standard), anti-E6 antibody incubation at 37° C., (1 hour), DAB detection using I-VIEW detection kit, hematoxylin II, (counterstain), and bluing reagent. At the completion of staining run, slides are post-processed by washing in 1% Dawn dishwashing detergent solution followed by a 3 minute tap water rinse. Slides are dehydrated through a series of alcohols and xylene, mounted and coverslipped. The IHC results demonstrate that the antibodies specific for the C-terminal end of E6 proteins bind and detect E6 proteins from high-risk HPV strains on HPV-containing tissues.

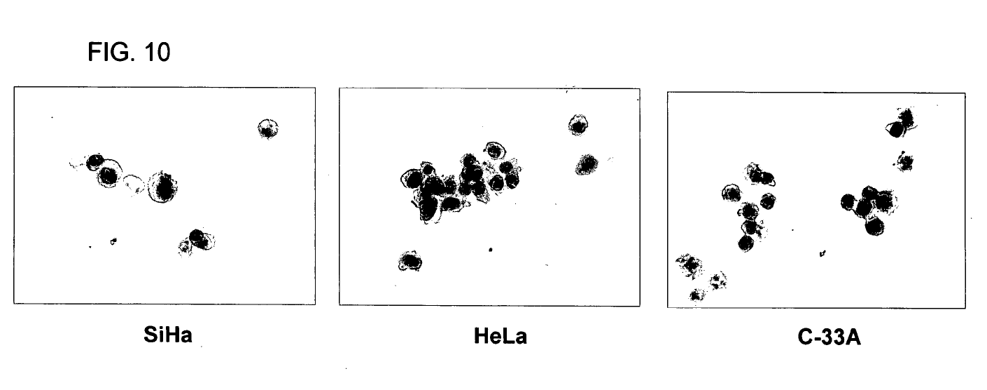

The IHC results are shown in FIG. 10. Cells were stained with a blue reagent. The anti-E6 mAb 1A9.1 was used to stain E6 in three cell lines: SiHa, Hela, which are both HPV positive, and C-33A, which is an HPV-negative cell line. mAb 1A9.1 used in combination with a brown dye DAB stained both SiHa and Hela cells expressing HPV E6 proteins but not the HPV-negative C-33A cells.

### Example 17

### E6 Protein of Oncogenic HPV16 Strain can be Detected in a Sandwich ELISA Via the “mAb-E6-mAb” Sandwich Approach

### (A) Abstract:

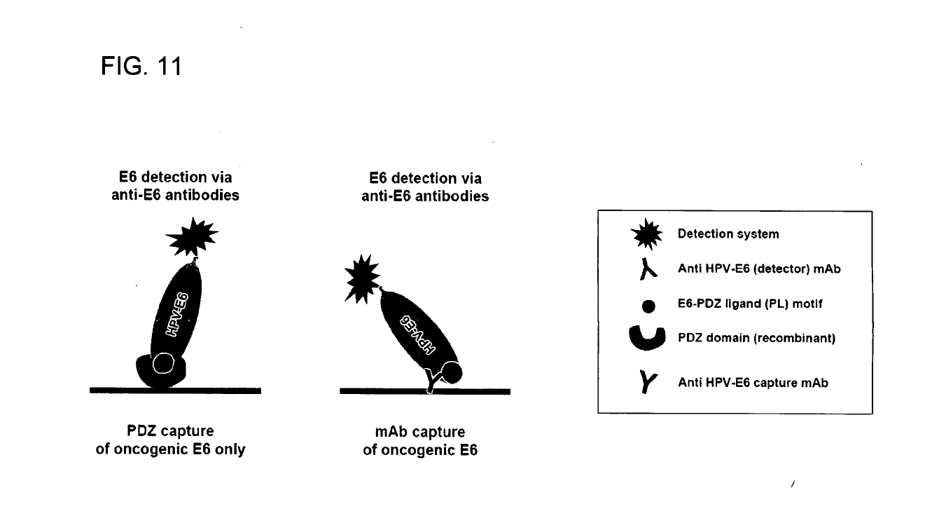

Experiments are described, in which the first anti-E6 capture antibody and the second anti-E6 detector antibody are used to selectively detect the presence of E6 protein in oncogenic HPV, for example, HPV16, infected cells via a sandwich ELISA. In this assay, E6 protein of an oncogenic HPV strain is captured by an anti-HPV16 E6 mAb, F126-6G6 mAb, which is coated on a solid substrate, such as a strip. The HPV E6 protein that is bound to the first capture mAb is then detected by the detector anti-E6 antibody, 4C6 (HPV16) alkaline phosphatase (AP) conjugated mAb and a detection system disclosed supra. This method is termed “mAb-E6-mAb” sandwich approach. This approach is in comparison with the “PDZ-E6-mAb” detection approach, in which a PDZ domain polypeptide disclosed hereinabove is coated onto a solid support to capture the E6 protein present in a sample. An anti-E6 mAb is then added to detect the E6 protein bound to the PDZ domain polypeptide. A scheme of the two approaches is shown in FIG. 11. The specific capturing of oncogenic E6 protein demonstrates that the antibody composition of the present invention can be applied for an E6 detection based diagnostic test for HPV infection and/or cervical cancer test.

### B) Methods:

Sandwich ELISA: Anti-E6 capture antibody (F126-6G6 mAb) or a PDZ domain polypeptide (for example, GST-MAGI1-PDZ1) is coated onto a test Strip at 5 ug/ml in PBS (100 ul/well) overnight at 4° C. Strips are washed with PBS and blocked with 200 ul PBS/2% BSA for 2 hours at 4° C. Cell lysates diluted in PBS/2% BSA are added and incubated at room temperature for 1 hour. To prepare the samples, negative cervical swab samples (NCLS) are lysed in 0.6 ml, spiked with 7,500 CaSki (HPV16) cells, B2B with addition of 0.4 ml B2deltaT. NCLS without spiking of the CaSki (HPV16) cells (OK) are used as the negative controls. After 3 washes with PBS, 0.15 ml of sample containing the oncogenic E6 is added onto the test Strip in PBS with 2% BSA as the blocking agent, and the strips are incubated at room temperature for 45 min. The strips are then washed 3 times with PBS and incubated with the detector anti-E6 antibody, 4C6 (HPV16) AP conjugated mAb, at the appropriate concentration in PBS/2% BSA at room temperature for 45 minutes, followed by anti-mouse IgG-HRP (Jackson Immuno Research). In a modified version of this sandwich assay, biotinylated reagents (e.g. biotinylated anti-E6 antibody) will be used as the detector antibody followed by streptavidin-HRP to further diminish background and to increase sensitivity.

For strip in which the PDZ domain polypeptide, e.g. GST-MAGI1-PDZ1, is coated, anti-GST-HRP (Pharmacia) may be added for detecting the binding of E6 to the PDZ domain polypeptide. After 5 washes with 50 mM Tris/0.2% Tween-20, the strips are incubated with 100 ul/well TMB substrate (Dako Industries). The colorimetric reaction is stopped at appropriate times (usually after 20 minutes) by addition of 100 ul of 0.1 M H₂SO₄, Readout/quantification is carried out via visual inspection and CAMAG reader. No heat treatment is applied.

In a variant of the sandwich ELISA disclosed hereinabove, cell lysates are preincubated with the second anti-E6 detector antibody at 2.5-5 ug/ml final concentration, for 1-2 hours at 4° C., prior to adding to the anti-E6 capture antibody coated strip.

### C) Results:

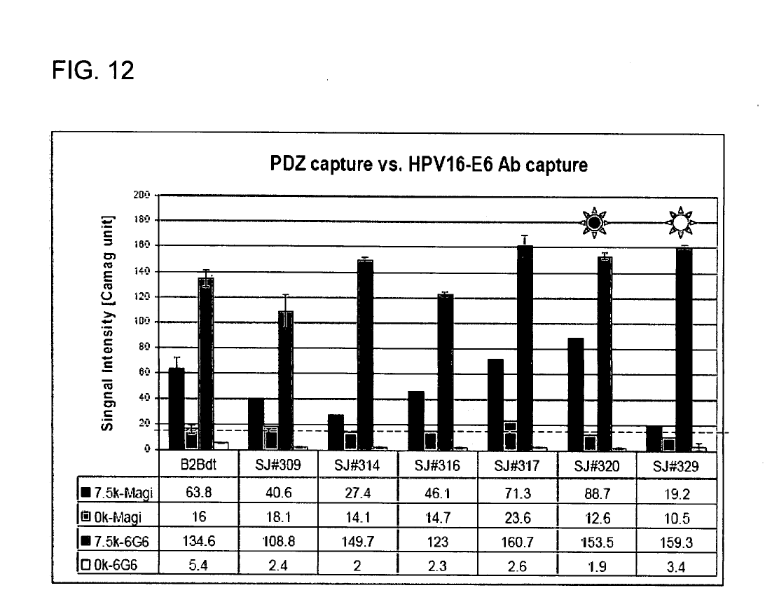

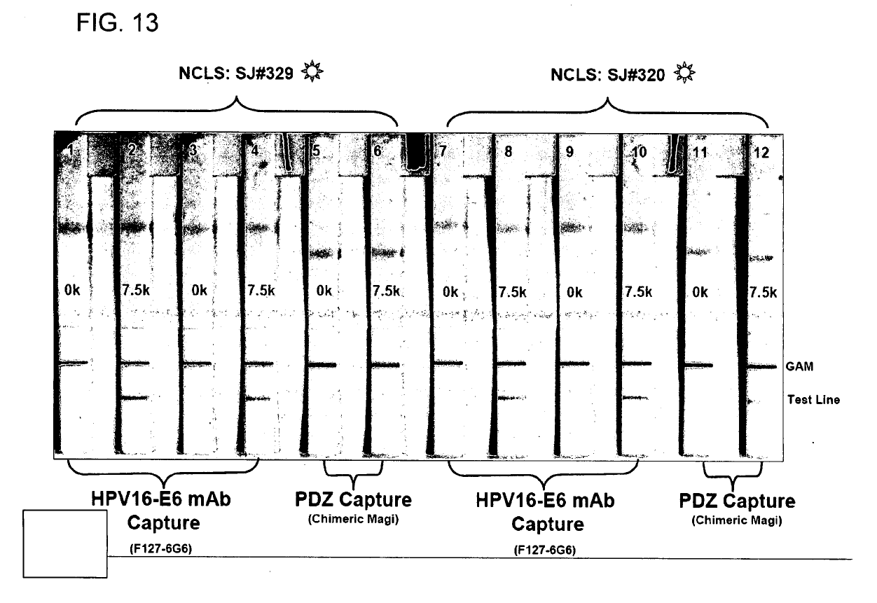

Results obtained from the mAb-E6-mAb sandwich ELISA assay are shown in FIGS. 12 and 13. For HPV16-E6, the “mAb-E6-mAb” sandwich approach results in a substantially improved signal-to-noise ratio, as compared to the “PDZ-E6-mAb” detection approach. The mAb-E6-mAb sandwich ELISA assay can detect E6 protein from a sample containing 7,500 HPV16 cells. In addition, the mAb-E6-mAb sandwich ELISA assay results in substantially decreased dampening with individual cervical swab samples. The “mAb-E6-mAb” sandwich approach has increased sensitivity in HPV16 singleplex detection.

### Example 18

### E6 Protein of Oncogenic HPV18 Strain can be Detected in a Sandwich ELISA Via the “mAb-E6-mAb” Sandwich Approach

### (A) Abstract:

Experiments are described, in which the first anti-E6 capture antibody and the second anti-E6 detector antibody are used to selectively detect the presence of E6 protein in oncogenic HPV18, infected cells via a sandwich ELISA. In this assay, E6 protein of an oncogenic HPV strain is captured by an anti-HPV18 E6 mAb, F82-3D4, which is coated on a solid substrate, such as a strip. The HPV E6 protein that is bound to the first capture mAb is then detected by the detector anti-E6 antibody, F82-5A2 (HPV18) alkaline phosphatase (AP) conjugated mAb and a detection system disclosed supra. This method is termed “mAb-E6-mAb” sandwich approach. This approach is in comparison with the “PDZ-E6-mAb” detection approach, in which a PDZ domain polypeptide disclosed hereinabove is coated onto a solid support to capture the E6 protein present in a sample. An anti-E6 mAb is then added to detect the E6 protein bound to the PDZ domain polypeptide. A scheme of the two approaches is shown in FIG. 12. The specific capturing of oncogenic E6 protein demonstrates that the antibody composition of the present invention can be applied for an E6 detection based diagnostic test for HPV infection and/or cervical cancer test.

### B) Methods:

Sandwich ELISA: Anti-E6 capture antibody (F82-3D4 mAb) or a PDZ domain polypeptide (for example, GST-MAGI1-PDZ1) is coated onto a test strip at 5 ug/ml in PBS (100 ul/well) overnight at 4° C. Strips are washed with PBS and blocked with 200 ul PBS/2% BSA for 2 hours at 4° C. Cell lysates diluted in PBS/2% BSA are added and incubated at room temperature for 1 hour. To prepare the samples, negative cervical swab samples (NCLS) are lysed in 0.6 ml, spiked with 10,000 HeLa (HPV18) cells, B2B with addition of 0.4 ml B2deltaT. NCLS without spiking of the HeLa (HPV18) cells (OK) are used as the negative controls. After 3 washes with PBS, 0.15 ml of sample containing the oncogenic E6 is added onto the strip in PBS with 2% BSA as the blocking agent, and the strips are incubated at room temperature for 45 min. The strips are then washed 3 times with PBS and incubated with the detector anti-E6 antibody, F82-5A2 (HPV18) AP conjugated mAb, at the appropriate concentration in PBS/2% BSA at room temperature for 45 minutes, followed by anti-mouse IgG-HRP (Jackson Immuno Research). In a modified version of this sandwich assay, biotinylated reagents (e.g. biotinylated anti-E6 antibody) will be used as the detector antibody followed by streptavidin-HRP to further diminish background and to increase sensitivity.

For strip in which the PDZ domain polypeptide, e.g. GST-MAGI1-PDZ1, is coated, anti-GST-HRP (Pharmacia) may be added for detecting the binding of E6 to the PDZ domain polypeptide. After 5 washes with 50 mM Tris/0.2% Tween-20, the strips are incubated with 100 ul/well TMB substrate (Dako Industries). The colorimetric reaction is stopped at appropriate times (usually after 20 minutes) by addition of 100 ul of 0.1 M H₂SO₄. Readout/quantification is carried out via visual inspection and CAMAG reader. No heat treatment is applied.

In a variant of the sandwich ELISA disclosed hereinabove, cell lysates are preincubated with the second anti-E6 detector antibody at 2.5-5 ug/ml final concentration, for 1-2 hours at 4° C., prior to adding to the anti-E6 capture antibody coated strip.

### C) Results:

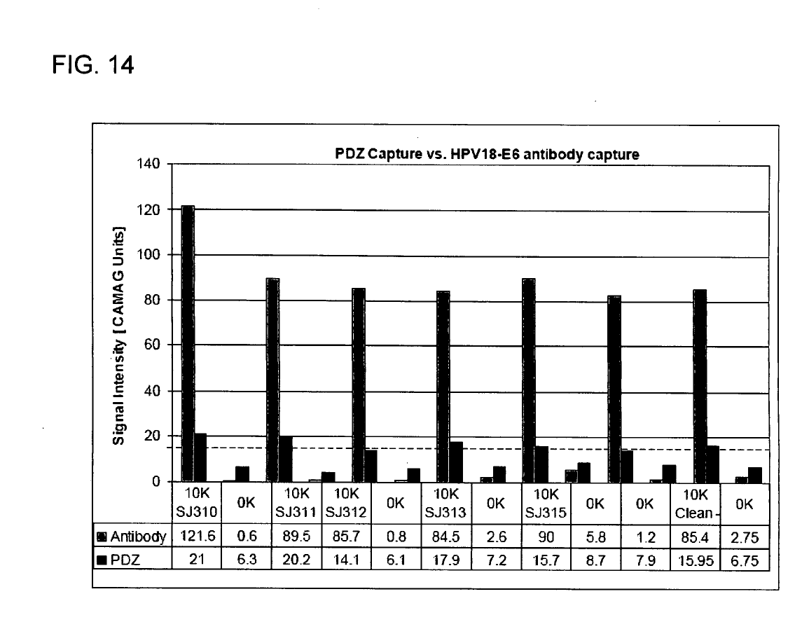

Results obtained from the mAb-E6-mAb sandwich ELISA assay are shown in FIG. 14. For HPV18-E6, the “mAb-E6-mAb” sandwich approach results in a substantially improved signal-to-noise ratio, as compared to the “PDZ-E6-mAb” detection approach. The mAb-E6-mAb sandwich ELISA assay can detect E6 protein from a sample containing 10,000 HPV18 cells. The “mAb-E6-mAb” sandwich approach has increased sensitivity in HPV18 singleplex detection.

### Example 19

### E6 Protein of Oncogenic HPV45 Strain can be Detected in a Sandwich ELISA Via the “mAb-E6-mAb” Sandwich Approach

The experiments and methods are as described in Examples 1 and 2, in which the first anti-E6 capture antibody and the second anti-E6 detector antibody are used to selectively detect the presence of E6 protein in oncogenic HPV45, infected cells via a sandwich ELISA. In this assay, E6 protein of an oncogenic HPV strain is captured by an anti-HPV45 E6 mAb, F154-4C5, which is coated on a solid substrate, such as a strip. The HPV E6 protein that is bound to the first capture mAb is then detected by the detector anti-E6 antibody, F82-3F3 (HPV45) alkaline phosphatase (AP) conjugated mAb and a detection system disclosed supra.

Anti-E6 capture antibody (F154-4C5 mAb) or a PDZ domain polypeptide (for example, GST-MAGI1-PDZ1) is coated onto a test strip at 5 ug/ml in PBS (100 μl/well) overnight at 4° C. Strips are washed with PBS and blocked with 200 ul PBS/2% BSA for 2 hours at 4° C. Cell lysates diluted in PBS/2% BSA are added and incubated at room temperature for 1 hour. To prepare the samples, negative cervical swab samples (NCLS) are lysed in 0.6 ml, spiked with 20,000 or 5,000 MS751 (HPV18 and HPV45 positive) cells, B2B with addition of 0.4 ml B2deltaT. NCLS without spiking of the MS751 cells are used as the negative controls. After 3 washes with PBS, 0.15 ml of sample containing the oncogenic E6 is added onto the strip in PBS with 2% BSA as the blocking agent, and the strips are incubated at room temperature for 45 min. The strips are then washed 3 times with PBS and incubated with the detector anti-E6 antibody, F82-3F3 (HPV45) AP conjugated mAb, at the appropriate concentration in PBS/2% BSA at room temperature for 45 minutes, followed by anti-mouse IgG-HRP (Jackson Immuno Research). In a modified version of this sandwich assay, biotinylated reagents (e.g. biotinylated anti-E6 antibody) will be used as the detector antibody followed by streptavidin-HRP to further diminish background and to increase sensitivity.

For strip in which the PDZ domain polypeptide, e.g. GST-MAGI1-PDZ1, is coated, anti-GST-HRP (Pharmacia) may be added for detecting the binding of E6 to the PDZ domain polypeptide. After 5 washes with 50 mM Tris/0.2% Tween-20, the strips are incubated with 100 ul/well TMB substrate (Dako Industries). The colorimetric reaction is stopped at appropriate times (usually after 20 minutes) by addition of 100 ul of 0.1 M H₂SO₄ Readout/quantification is carried out via visual inspection and CAMAG reader. No heat treatment is applied.

In a variant of the sandwich ELISA disclosed hereinabove, cell lysates are preincubated with the second anti-E6 detector antibody at 2.5-5 ug/ml final concentration, for 1-2 hours at 4° C., prior to adding to the anti-E6 capture antibody coated strip.

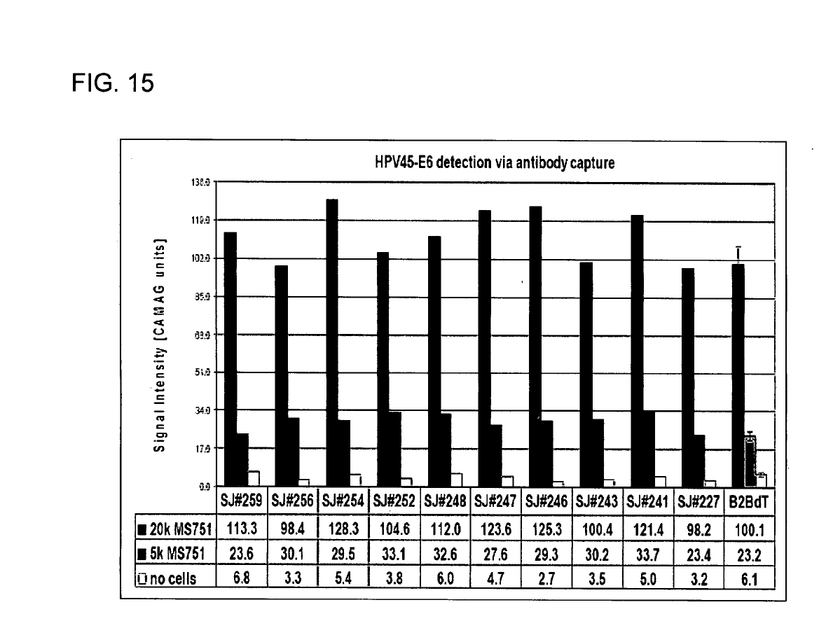

Results obtained from the mAb-E6-mAb sandwich ELISA assay are shown in FIG. 15. For HPV45-E6, the “mAb-E6-mAb” sandwich approach results in a substantially improved signal-to-noise ratio, as compared to the “PDZ-E6-mAb” detection approach. The mAb-E6-mAb sandwich ELISA assay can detect E6 protein from a sample containing 20,000 and 5,000 HPV18 and HPV45 positive cells. In addition, no NCLS specific dampening is observed. The “mAb-E6-mAb” sandwich approach has increased sensitivity in HPV45 singleplex detection.

### Example 20

### E6 Proteins of Oncogenic HPV16 and HPV18 can be Detected in a Multiplex Sandwich Assay Via the “mAb-E6-mAb” Sandwich Approach

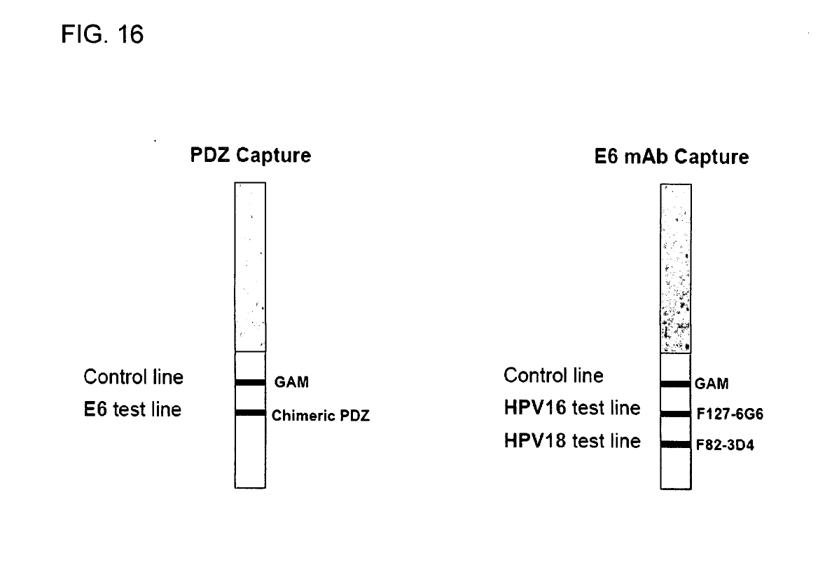

Experiments are described, in which the first anti-E6 capture antibody and the second anti-E6 detector antibody are used to selectively detect the presence of E6 proteins from more than one oncogenic HPV strain, for example, HPV16 and HPV18, infected cells via a sandwich ELISA. In this assay, E6 proteins of HPV16 and HPV18 are captured by an anti-HPV16 E6 mAb, F127-6G6, and an anti-HPV18 E6 mAb, F82-3D4, respectively. The two capture antibodies are coated on a solid substrate, such as a strip. The HPV E6 proteins that are bound to the first capture mAb are then detected by the detector anti-E6 antibodies, 4C6 (HPV16) and F82-5A2 (HPV18) alkaline phosphatase (AP) conjugated mAb cocktail and a detection system disclosed supra. HPV type-specific E6 detection allows for an E6 strip test in which different HPV types, e.g. HPV16 and HPV18, are detected as two distinct test lines on one strip. In contrast, this HPV type specific E6 detection can not be easily achieved via E6 detection by a PDZ domain polypeptide. A scheme of the multiplex HPV type specific E6 protein detection using the method of the present invention is shown in FIG. 16.

The method of carrying out the sandwich ELISA on a strip is as described in Examples 17-19 herein. Anti-E6 capture antibodies (F127-6G6 mAb and F82-3D4 mAb) are coated onto a test strip at 5 ug/ml in PBS (100 ul/well) overnight at 4° C. The strips are washed with PBS and blocked with 200 ul PBS/2% BSA for 2 hours at 4° C. Cell lysates diluted in PBS/2% BSA are added and incubated at room temperature for 1 hour. To prepare the samples, negative cervical swab samples (NCLS) are lysed in 0.6 ml, spiked with 5,000 HeLa (HPV18) or CaSki (HPV16) cells or both HeLa and CaSki cells, B2B with addition of 0.4 ml B2deltaT. NCLS without spiking of any cells are used as the negative controls. After 3 washes with PBS, 0.15 ml of sample containing the oncogenic E6 is added onto the strip in PBS with 2% BSA as the blocking agent, and the strips are incubated at room temperature for 45 min. The strips are then washed 3 times with PBS and incubated with the detector anti-E6 antibodies, 4C6 (HPV16) and F82-5A2 5A2 (HPV18) AP conjugated mAb cocktail, at the appropriate concentration in PBS/2% BSA at room temperature for 45 minutes, followed by anti-mouse IgG-HRP (Jackson Immuno Research). In a modified version of this sandwich assay, biotinylated reagents (e.g. biotinylated anti-E6 antibody) will be used as the detector antibody followed by streptavidin-HRP to further diminish background and to increase sensitivity. After 5 washes with 50 mM Tris/0.2% Tween-20, the strips are incubated with 100 ul/well TMB substrate (Dako Industries). The colorimetric reaction is stopped at appropriate times (usually after 20 minutes) by addition of 100 ul of 0.1 M H₂SO₄. Readout/quantification is carried out via visual inspection and CAMAG reader. No heat treatment is applied.

In a variant of the sandwich ELISA disclosed hereinabove, cell lysates are preincubated with the second anti-E6 detector antibody cocktail at 2.5-5 ug/ml final concentration, for 1-2 hours at 4° C., prior to adding to the anti-E6 capture antibodies-coated strip.

Results obtained from the multiplex mAb-E6-mAb sandwich ELISA assay are shown in FIGS. 17 and 18. Capture of E6 protein via HPV type specific mAb allows for E6 typing of different HPV strains (for example, HPV16+HPV18). In addition, the HPV16/HPV18 mAb detector cocktail does not result in an enhanced background or a reduced signal as compared to E6 singleplex detection, which refers to detection of E6 protein from a single HPV strain. The mAb-E6-mAb sandwich ELISA assay can detect E6 proteins from a sample containing 5,000 HPV18 and/or HPV16 positive cells.

### Example 21

### The Multiplex “mAb-E6-mAb” Sandwich Approach has Low False Positive Rate of Detecting E6 Protein

Experiments are described, in which the first anti-E6 capture antibody and the second anti-E6 detector antibody are used to selectively detect the presence of E6 proteins from more than one oncogenic HPV strain, for example, HPV16 and HPV18, infected cells via a sandwich ELISA. In this assay, E6 proteins of HPV16 and HPV18 are captured by an anti-HPV16 E6 mAb, F127-6G6, and an anti-HPV18 E6 mAb, F82-3D4, respectively. The two capture antibodies are coated on a solid substrate, such as a strip. The HPV E6 proteins that are bound to the first capture mAb are then detected by the detector anti-E6 antibodies, 4C6 (HPV16) and F82-5A2 (HPV18) alkaline phosphatase (AP) conjugated mAb cocktail and a detection system disclosed supra. HPV type-specific E6 detection allows for an E6 strip test in which different HPV types, e.g. HPV16 and HPV18, are detected as two distinct test lines on one strip.

Anti-E6 capture antibodies (F127-6G6 mAb and F82-3D4 mAb) are coated onto a test strip at 5 ug/ml in PBS (100 μl/well) overnight at 4° C. The strips are washed with PBS and blocked with 200 ul PBS/2% BSA for 2 hours at 4° C. Cell lysates from 60 individual HPV negative cervical swab samples (NCLS) diluted in PBS/2% BSA are added to the strip and incubated at room temperature for 1 hour. After 3 washes with PBS, 0.15 ml of each NCLS sample is added onto the strip in PBS with 2% BSA as the blocking agent, and the strips are incubated at room temperature for 45 min. The strips are then washed 3 times with PBS and incubated with the detector anti-E6 antibodies, 4C6 (HPV16) and F82-5A2 (HPV18) AP conjugated mAb cocktail, at the appropriate concentration in PBS/2% BSA at room temperature for 45 minutes, followed by anti-mouse IgG-HRP (Jackson Immuno Research). In a modified version of this sandwich assay, biotinylated reagents (e.g. biotinylated anti-E6 antibody) will be used as the detector antibody followed by streptavidin-HRP to further diminish background and to increase sensitivity. After 5 washes with 50 mM Tris/0.2% Tween-20, the strips are incubated with 100 ul/well TMB substrate (Dako Industries). The colorimetric reaction is stopped at appropriate times (usually after 20 minutes) by addition of 100 ul of 0.1 M H₂SO₄ Readout/quantification is carried out via visual inspection and CAMAG reader. No heat treatment is applied.

Results obtained from the multiplex mAb-E6-mAb sandwich ELISA assay on the 60 NCLS are shown in FIG. 19. The signals in CAMAG units for all 60 negative cervical swab samples are below the limit of visibility to the eye, indicating that there is 0 positive sample out of the 60 negative cervical swab samples. The results show that there is no HPV16/HPV18 false positive on 60 negative samples tested. The false positive rate is less than 1.7%.

### Example 22

### E6 Proteins of Oncogenic HPV16, HPV18, and HPV45 can be Detected in a Multiplex Sandwich Assay Via the “mAb-E6-mAb” Sandwich Approach

Experiments are described, in which the first anti-E6 capture antibody and the second anti-E6 detector antibody are used to selectively detect the presence of E6 proteins from more than one oncogenic HPV strain, for example, HPV16, HPV18, and HPV45, infected cells via a sandwich ELISA. In this assay, E6 proteins of HPV16, HPV18, and HPV45 are captured by an anti-HPV16 E6 mAb, F127-6G6, an anti-HPV18 E6 mAb, F82-3D4, and an anti-HPV45 E6 mAb, F82-3F3, respectively. The three capture antibodies are coated on a solid substrate, such as a strip. The HPV E6 proteins that are bound to the first capture mAb are then detected by the detector anti-E6 antibodies, 4C6 (HPV16), F82-5A2 (HPV18) and 6F4 (HPV45) alkaline phosphatase (AP) conjugated mAb cocktail and a detection system disclosed supra. HPV type-specific E6 detection allows for an E6 strip test in which different HPV types, e.g. HPV16, HPV18, and HPV45, are detected as three distinct test lines on one strip.

The method of carrying out a multiplex sandwich ELISA on a strip is as described in Example 4 herein. Anti-E6 capture antibodies (F127-6G6 mAb, F82-3D4 mAb, and F82-3F3 mAb) are coated onto a test strip at 5 ug/ml in PBS (100 μl/well) overnight at 4° C. The strips are washed with PBS and blocked with 200 ul PBS/2% BSA for 2 hours at 4° C. Cell lysates diluted in PBS/2% BSA are added and incubated at room temperature for 1 hour. To prepare the samples, negative cervical swab samples (NCLS) are lysed in 0.6 ml, spiked with 5,000 HeLa (HPV18+), 5,000 CaSki (HPV16+) cells, or 5,000 MS751 (HPV45+) cells, or all three cell lines, B2B with addition of 0.4 ml B2deltaT. NCLS without spiking of any cells are used as the negative controls. After 3 washes with PBS, 0.15 ml of sample containing the oncogenic E6 is added onto the strip in PBS with 2% BSA as the blocking agent, and the strips are incubated at room temperature for 45 min. The strips are then washed 3 times with PBS and incubated with the detector anti-E6 antibodies, 4C6 (HPV16), F82-5A2 (HPV18), and 6F4 (HPV45)-AP conjugated mAb cocktail, at the appropriate concentration in PBS/2% BSA at room temperature for 45 minutes, followed by anti-mouse IgG-HRP (Jackson Immuno Research). In a modified version of this sandwich assay, biotinylated reagents (e.g. biotinylated anti-E6 antibody) will be used as the detector antibody followed by streptavidin-HRP to further diminish background and to increase sensitivity. After 5 washes with 50 mM Tris/0.2% Tween-20, the strips are incubated with 100 ul/well TMB substrate (Dako Industries). The colorimetric reaction is stopped at appropriate times (usually after 20 minutes) by addition of 100 ul of 0.1 M H₂SO₄. Readout/quantification is carried out via visual inspection and CAMAG reader. No heat treatment is applied.

In a variant of the sandwich ELISA disclosed hereinabove, cell lysates are preincubated with the second anti-E6 detector antibody cocktail at 2.5-5 ug/ml final concentration, for 1-2 hours at 4° C., prior to adding to the anti-E6 capture antibodies-coated strip.

Results obtained from the multiplex mAb-E6-mAb sandwich ELISA assay are shown in FIG. 20. E6 proteins from HPV16, HPV18, and HPV45 are detected as three distinct lines on the strip. Capture of E6 protein via HPV type specific mAb allows for E6 typing of different HPV strains (for example, HPV16, HPV18, and HPV45). In addition, the HPV16/HPV18/HPV45 mAb detector cocktail does not result in an enhanced background or a reduced signal as compared to E6 singleplex detection, which refers to detection of E6 protein from a single HPV strain. The mAb-E6-mAb sandwich ELISA assay can detect E6 proteins from a sample containing 5,000 HPV16, HPV18, and/or HPV45 positive cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### REFERENCES

1. PDZ Domains: Structural modules for protein complex assembly. (2202) J. of Biol. Chem. 277: 5699-5702.
2. HPV E6 specifically targets different cellular pools of its PDZ domain-containing tumor suppressor substrates for proteasome-mediated degradation (2004) Oncogene 23: 8033-8039.
3. Structural and functional analysis of E6 oncoprotein: Insights in the molecular pathways of human papollomavirus-mediated pathogenesis. (2006) Molecular Cell 21: 1-14.
4. Human papillomavirus and cancer: the epidemiological evidence. (2000) J. of Clin. Vir. 19: 1-5.
5. The PDZ ligand domain of the human papillomavirus type 16 E6 protein is required for E6's induction of epithelial hyperplasia in vivo. (2003) J. of Vir. 77: 6957-6964.
6. Dell et al. Contributions in the domain of cancer research: Review Human papillomaviruses and their role in cervical cancer. Cell. Mol. Life. Sci. 2001, 58:1923-1942.
7. Burd et al., Human papillomavirus and cervical cancer. Clin Microbiol Rev. 2003, 16:1-17.
8. Woodman et al., The natural history of cervical HPV infection: unresolved issues. Nature Reviews 2007, 7:11-22.
9. Mouritsen, S. a. E., Henrik. Inducing antibody response against self-proteins with the aid of foreign T-cell epitopes. (1994).
10. European patent EPO0752886 B1
11. McMahon, M., Murphy, T. F., Kyd, J. & Thanavala, Y. Role of an immunodominant T cell epitope of the P6 protein of nontypeable Haemophilus influenzae in murine protective immunity. Vaccine 23, 3590-6 (2005).
12. Gore, M. M., Kolaskar, Ashok, Dewasthaly, S., S., Kulkarani-kale, Urmila, D., Sawant, Sangeeta. Chimeric T helper-B cell peptide vaccine for Japanese encephalitis virus (2007).
13. US patent 20080075738 A1
14. Dakappagari, N. K. et al. A chimeric multi-human epidermal growth factor receptor-2 B cell epitope peptide vaccine mediates superior antitumor responses. J Immunol 170, 4242-53 (2003).
15. Simone Niederhäuser, et al.: A Gag peptide encompassing B- and T-cell epitopes of the caprine arthritis encephalitis virus functions as modular carrier peptide Pages 82-90.
16. S C Hsu, D Chargelegue, O E Obeid, and M W Steward Synergistic effect of immunization with a peptide cocktail inducing antibody, helper and cytotoxic T-cell responses on protection against respiratory syncytial virus J Gen Virol 1999 80: 1401-1405.
17. Novel T-cell epitopes of ovalbumin in BALB/c mouse: Potential for peptide-immunotherapy Pages 203-208 Marie Yang, Yoshinori Mine, Biochemical and Biophysical Research Communications 2009 Elsevier Inc.
18. England, R. D., Kullberg, M. C., Corvette, J. L. & Berzofsky, J. A. Molecular analysis of a heteroclitic T cell response to the immunodominant epitope of sperm whale myoglobin. Implications for peptide partial agonists. J Immunol 155, 4295-306 (1995).
19. U.S. Pat. No. 5,114,713 on P. falciparum CS-peptides as universal T-cell epitope

What is claimed is:

1-162. (canceled)

163. An antibody composition for detecting E6 protein of a plurality of HPV strains in a sample, the composition comprising a first antibody and a second antibody, wherein the first antibody binds to an E6 protein of a first HPV strain, and the second antibody binds to an E6 protein of a second HPV strain wherein the first and second antibodies are arranged on a solid support such that the E6 protein of the first HPV strain is detectable at a first region on the solid support and the E6 protein of the second HPV strain is detectable at a second region on the solid support, and wherein the first and second regions comprise different sets of antibodies.

164. The antibody composition of claim 163, wherein the first HPV strain is an oncogenic HPV strain.

165. (canceled)

166. The antibody composition of claim 163, wherein the first HPV strain is selected from the group consisting of HPV strains 16, 18, 26, 30, 31, 33, 35, 39, 45, 51, 52, 53, 56, 58, 59, 66, 68, 69, 73, and 82.

167. The antibody composition of claim 163, wherein the first HPV strain is HPV-6 or HPV-11.

168. The antibody composition of claim 163, wherein the first antibody specifically binds to E6 protein of HPV strain 16, 18, 31, 33, 45, 52 or 58.

169. (canceled)

170. (canceled)

171. The antibody composition of claim 163, wherein the first and the second antibodies bind to E6 proteins of HPV-16, HPV-18, HPV-45 or a combination thereof.

172. (canceled)

173. The antibody composition of claim 163, wherein the sample is a cervical scrape, cervical biopsy, cervical lavage, blood or urine.

174. (canceled)

175. The antibody composition of claim 163, wherein the first and the second antibodies are monoclonal.

176. The antibody composition of claim 163, wherein the first or second antibody binds to the N-terminus of an E6 protein and further comprising a third antibody that binds to the C-terminus of the E6 protein.

177. The antibody composition of claim 163, wherein the first or second antibody binds to the C-terminus of an E6 protein and further comprising a third antibody that binds to the N-terminus of the E6 protein.

178. The antibody composition of claim 176, wherein the third antibody is labeled.

179. The antibody composition of claim 176, wherein the third antibody is conjugated to an enzyme.

180-184. (canceled)

185. The antibody composition of claim 163, wherein the antibody composition enhances signal-to-noise ratio of detecting an oncogenic E6 protein as compared to using a PDZ domain containing polypeptide for the detection of an oncogenic E6 protein in a sample.

186. (canceled)

187. The antibody composition of claim 163, wherein the antibody composition can be used to detect oncogenic E6 protein in a sample with a false positive rate that is less than 1.7%.

188. (canceled)

189. The antibody composition of claim 163, wherein two or more antibodies recognizing different epitopes of E6 protein are used for capture and/or detection of the E6 protein.

190. A diagnostic kit for detection of a plurality of strains of HPV in a sample, the kit comprising an antibody composition of claim 163.

191. The diagnostic kit of claim 190, wherein the kit detects E6 protein of at least one oncogenic HPV strain.

192. (canceled)

193. The diagnostic kit of claim 190, wherein the kit detects E6 proteins of HPV-16, HPV-18, HPV-45 or a combination thereof.

194. The diagnostic kit of claim 190 further comprising a strip on which the first and the second antibodies bind to E6 protein of at least one oncogenic HPV strain.

195. The diagnostic kit of claim 190, wherein the kit further contains reagents for detection of the second antibody that is bound to an E6 protein by an enzyme-linked immunosorbent assay (ELISA).

196. A method for detecting E6 protein of a plurality of HPV strains in a sample, comprising:
(a) contacting a first antibody which specifically binds to a first E6 protein of at least one first strain of HPV with the sample;
(b) contacting a second antibody which specifically binds to a second E6 protein of at least one second strain of HPV with the sample; and
(c) detecting binding of the first or second antibody to the first or second E6 protein, thereby detecting the first or second E6 protein in the sample;
wherein the first and second antibodies are arranged on a solid support such that the first E6 protein is detectable at a first region on the solid support and the second E6 protein is detectable at a second region on the solid support, and wherein the first and second regions comprise different sets of antibodies.

197. The method of claim 196, wherein the first HPV strain is an oncogenic HPV strain.

198. (canceled)

199. The method of claim 196, wherein the first HPV strain is selected from the group consisting of HPV strains 16, 18, 26, 30, 31, 33, 35, 39, 45, 51, 52, 53, 56, 58, 59, 66, 68, 69, 73, and 82.

200-225. (canceled)

226. A method for ameliorating an HPV disease, comprising administering to a subject in need thereof an effective amount of an antibody which specifically binds an HPV E6 protein.

227-240. (canceled)

241. The antibody composition of claim 163, wherein the solid support is a strip.

242. The antibody composition of claim 163, wherein the first region and the second region each forms a test line across the solid support.

243. The antibody composition of claim 163, wherein the composition is capable of detecting E6 proteins from a sample containing less than about 10,000 HPV positive cells.

244. The antibody composition of claim 163, wherein the first and second antibodies are not separated in different vessels.

245. The antibody composition of claim 163, wherein the solid support is not a well.

246. The antibody composition of claim 163, wherein the antibody composition can be used to detect oncogenic E6 protein in a sample with a false positive rate that is less than 10%.

247. The antibody composition of claim 163, wherein the antibody composition can be used to detect oncogenic E6 protein in a sample with a specificity of at least about 85%.

248. The antibody composition of claim 176, wherein the E6 protein or a portion of the E6 protein is bound to the first antibody prior to contacting the third antibody.

249. The antibody composition of claim 177, wherein the E6 protein or a portion of the E6 protein is bound to the second antibody prior to contacting the third antibody.
